(19) United States
(12) Patent Application Publication
(10) Pub. No.: US 2026/0038679 A1
(43) Pub. Date: Feb. 5, 2026

(54) Systems, Methods, and Apparatuses for Producing and Packaging Medical Fluids

(71) Applicant: DEKA Products Limited Partnership, Manchester, NH (US)

(72) Inventors: Jason M. Overson, Manchester, NH (US); Gillian R. Cohen, Kalamazoo, MI (US); Robert H. Lawler, JR., Manchester, NH (US); John J. Wight, JR., Manchester, NH (US); Robert Piccirillo, Salem, NH (US); Matthew Handley, Manchester, NH (US); Blake Lagasse, Manchester, NH (US); Caleb D. Kase, Manchester, NH (US); Kunj P. Patel, Manchester, NH (US); Mohammed Aun Siddiqui, San Carlos, CA (US); Stepan Vartanov, Manchester, NH (US); Michael Quilici, Concord, NH (US); Andrew A. Schnellinger, Merrimack, NH (US); Rajesh G. Nagula, Nashua, NH (US); Priya Vijayakumar, Northville, MI (US); Kayli M. Hill, Loudon, NH (US)

(21) Appl. No.: 19/223,870

(22) Filed: May 30, 2025

Related U.S. Application Data
(60) Provisional application No. 63/677,620, filed on Jul. 31, 2024.
(60) Provisional application No. 63/775,493, filed on Mar. 21, 2025.
(60) Provisional application No. 63/775,457, filed on Mar. 21, 2025.
(60) Provisional application No. 63/775,455, filed on Mar. 21, 2025.

(51) Int. Cl. G16H 40/63; A61J 1/10; G06V 10/14; G06V 10/25; G06V 10/764; G06V 10/82; H04N 23/56; H04N 23/90

(52) U.S. Cl. CPC G16H 40/63; A61J 1/10; G06V 10/14; G06V 10/25; G06V 10/764; G06V 10/82; H04N 23/56; H04N 23/90; G06V 2201/03

(57) ABSTRACT

A particulate inspection system may comprise a reservoir rest body having a pattern of light and dark regions. The system may further comprise a rest body backlight. The system may further comprise an external illuminator. The system may further comprise a vision assembly opposite the rest body. The vision assembly may include at least one imager. Each imager may have a field of view encompassing at least a portion of rest body. The system may further comprise a controller in data communication with each of the at least one imager. The controller may be configured to command capture of a series of images from each imager, receive the images, process the images into processed images, detect regions of interest within the processed images, analyze the regions of interest, and classify the regions of interest as one of a bubble and something other than bubble.

### CROSS-REFERENCE TO RELATED APPLICATIONS

The present application claims the benefit of U.S. Provisional Application Ser. No. 63/677,620, entitled System for and Method of Detecting and Differentiating Particulates, filed Jul. 31, 2024 (Attorney Docket No. 00101.00507.AB522), and claims the benefit of U.S. Provisional Application Ser. No. 63/775,493, entitled Systems, Methods, and Apparatuses for Producing and Packaging Medical Fluids, filed Mar. 21, 2025 (Attorney Docket No. 00101.00465.AB670), and claims the benefit of U.S. Provisional Application Ser. No. 63/775,457, entitled Systems, Methods, and Apparatuses for Producing and Packaging Medical Fluids, filed Mar. 21, 2025 (Attorney Docket No. 00101.00336.AA849), and claims the benefit of U.S. Provisional Application Ser. No. 63/775,455, entitled Systems, Methods, and Apparatuses for Producing and Packaging Medical Fluids, filed Mar. 21, 2025 (Attorney Docket No. 00101.00335.AA829) each of the above being incorporated by reference herein in their entireties.

### STATEMENT REGARDING FEDERALLY SPONSORED RESEARCH OR DEVELOPMENT

This invention was made with Government support under Agreement N00014-23-9-0001, awarded by The Office of Naval Research. The Government has certain rights in the invention.

### BACKGROUND

### Field of Disclosure

This disclosure relates to medical fluids. More specifically, this disclosure relates to the generation and packaging of medical fluids.

### Description of Related Art

Almost every hospitalized patient is administered saline or a saline based solution. As a result, the quantity of saline solution consumed is very large. More than a billion bags of saline are used per year in the US alone. Despite the demand, there are only a small number of different saline manufactures which provide this solution for the US market. Unfortunately, manufacturing challenges which limit production from one manufacturer can and do cause shortages of saline in the United States. Compounding the issue, these manufactures have uneven market share in regards to all bagged saline products. For instance, 50% of 250 ml or smaller saline bags are provided by a single manufacture. As a result, when such a manufacturer faces production problems, the impact on the availability of that particular type of bag is much greater.

Most recently, the media spotlight has been shown on delays caused in the wake of hurricane Maria which have led to a shortage of small volume saline bags. According to the American Society of Health-System Pharmacists, shortages for large volume bags and bags of saline for irrigation purposes also currently exist. An alternative means of producing medical fluid bags which may perhaps be locatable in the institution using the bag would be desirable.

### SUMMARY

In accordance with another example embodiment of the present disclosure a method of mixing fluid in a medical bag may comprise placing a bag in a cradle of a bag retention assembly. The method may further comprise grasping ports of the bag with a grasper of the bag retention assembly. The method may further comprise closing a set of doors of the bag retention assembly. The method may further comprise rotating the bag retention assembly in opposing directions between preset pairs of rotational orientations. The method may further comprise dwelling in each preset rotational orientation for a dwell interval specific to each pair of positions.

In some embodiments, the method may further comprise dissolving a solid concentrate selected from a list consisting of powder, lyophilized medical agent, crystalline concentrate, salt concentrate, sodium chloride concentrate, normal saline concentrate, half normal saline concentrate, sugar concentrate, dextrose concentrate, a saline and sugar solution concentrate, D5W concentrate, peritoneal dialysate concentrate, hemodialysis dialysate concentrate, Ringer's solution concentrate, lactated Ringer's solution concentrate, and Hartmann's solution concentrate. In some embodiments, the method may further comprise waiting a predefined period of time and analyzing the medical bag for the presence of particulates. In some embodiments, the method further may comprise reading an indicium on the medical bag and selecting a motion profile based on data read from the indicium. The motion profile may define the preset pairs of rotational orientations. In some embodiments, rotating the bag retention assembly may comprise rotating the bag retention assembly at a rotation rate specific to each pair of positions. In some embodiments, rotating the bag retention assembly may comprise rotating the bag retention assembly between each pair of preset positions a number of times specific to each pair of positions. In some embodiments, rotating the bag retention assembly may comprise inverting the bag retention assembly at least once. In some embodiments, each rotational orientation in each pair of rotational positions may be equal in magnitude from a starting orientation and opposite in direction. In some embodiments, closing the set of doors on the medical bag may comprise depressing a central region of the medical bag and displacing fluid laterally within the medical bag. In some embodiments, closing the set of doors on the medical bag may comprise contacting the medical bag with a gripping material on a surface of the doors. In some embodiments, the method may further comprise collecting a plurality of images of the medical bag and analyzing the plurality of images for indications of undissolved concentrate. The method may further comprise rotating the bag retention assembly in opposing directions at between at least one preset pair of rotational orientations when at least one indication of undissolved concentrate is detected. In some embodiments, closing the doors may comprise monitoring the position of the doors and generating a fault notification with a controller when at least one of the doors reaches an overly closed position.

In accordance with another example embodiment of the present disclosure a medical reservoir mixing assembly may comprise a bag retention assembly including a cradle and a reservoir holder assembly. The mixing assembly may further comprise a rotary actuator coupled the bag retention assembly. The mixing assembly may further comprise a rotation sensor configured to output a data signal indicative of the rotational orientation of the bag retention assembly. The mixing assembly may further comprise a controller in data communication with the rotation sensor and the rotary actuator and configured to orchestrate displacement of the bag retention assembly via the rotary actuator in opposing rotational directions between preset pairs of rotational orientations. The controller may be further configured to wait for a dwell timer to elapse at each rotational orientation.

In some embodiments, the reservoir holder assembly may include a set of doors each coupled to at least one door actuator. In some embodiments, the reservoir holder assembly may include a set of doors. Each door may have an inner face and an exterior face. At least a portion of the inner face may be covered with a gripping material. In some embodiments, the reservoir holder assembly may include a set of doors displaceable between an open state and a closed state. Each door may include a set of projections. The projections of the doors may interdigitate when the doors are in a closed state. In some embodiments, the projections may be more proximal a reservoir rest surface of the cradle than a section of the door from which they extend when the doors are in the closed state. In some embodiments, each door may include three projections. All edges of each projection may be rounded. Each projection may include a base region. There may be a rounded transition between the base region and the end of the door from which the projection projects. The base region may taper thinner as distance from the end of the door from which the projection projects increases. Each projection may further include a rounded transition to at the unsupported end of the projection. The tip of the nub may be rounded. In some embodiments, the reservoir holder assembly may include a set of IV bag port grippers actuatable between an open state and a closed state. In some embodiments, the mixing assembly may further comprise a reservoir indicium reader. The controller may be in data communication with the indicium reader and may select a motion profile defining the preset pairs rotational orientations based on data from the indicium reader. In some embodiments, the controller may be further configured to orchestrate displacement of the bag retention assembly at a rotation rate specific to each pair of positions of the preset pairs of positions. In some embodiments, the controller may be further configured to orchestrate displacement of the bag retention assembly to each preset pair of positions a number of times specific to each pair of positions. In some embodiments, one of the preset pairs of positions may orient the bag retention assembly in an inverted position. In some embodiments, each rotational orientation in each pair of rotational positions may be equal in magnitude from a starting orientation and opposite in direction. In some embodiments, the cradle may be illuminated and may include a pattern of light and dark regions. The mixing assembly may further comprise an external illuminator and at least one imager with a field of view at least partially encompassing the bag retention assembly.

In accordance with another example embodiment of the present disclosure a particulate inspection system for detecting and classifying contents of interest within a medical reservoir may comprise a cradle including a reservoir rest and a reservoir retainer. The cradle may include a backlight. The rest body may be in an illumination field of the backlight and may include a pattern of light and dark regions. The system may further comprise an external illuminator including at least light emitter. The system may further comprise a vision assembly disposed opposite the cradle. The vision assembly may include at least one imager having a field of view encompassing at least a portion of the cradle. The system may further comprise a controller in data communication with each of the at least one imager. The controller may be configured to command capture of a series of images from each of the at least one imager, receive the images, pre-process the images into processed images, detect regions of interest within the processed images, analyze the regions of interest, and classify the regions of interest into at least a first content type and second content type based on the analysis.

In some embodiments, the system may further comprise a rotary actuator coupled to the cradle. In some embodiments, the backlight may emit white light. In some embodiments, the pattern may be a chessboard pattern. In some embodiments, the reservoir retainer may be a set of IV bag port graspers actuatable between an open state and port retaining state. In some embodiments, the external illuminator may be an underlight. In some embodiments, the at least one light emitter may include a plurality of LEDs electrically coupled in series. In some embodiments, each of the at least one light emitter may be a collimated light emitter. In some embodiments, each of the at least one light emitter may emit light in a narrow beam angle less than 20°. In some embodiments, the external illuminator may include a polarizer. In some embodiments, each of the at least one image may include a polarizing filter on a lens thereof. In some embodiments, the vision system may includes a plurality of imagers. Each of the field of view of each imager may overlap the field of view of at least one other imager of the vision system. In some embodiments, the optical axis or viewing axis each of the at least one imager may be oriented at an angle 30-35° from a plane extending perpendicular to the height axis of the cradle. In some embodiments, the controller may be further configured to assign each region of interest in each of the processed images to one or more track. Each of the one or more track may define the displacement of an associated region of interest over the series of images. In some embodiments, for each track, the controller may be configured to extract an extracted portion of every processed image in which the associated region of interest is detected. The extracted portion may include the associated region of interest. The controller may be configured to analyze the regions of interest by analyzing the extracted portions of the processed images for each track. In some embodiments, the controller may be configured to analyze the regions of interest with a convolutional neural network. In some embodiments, the controller may be configured generate a reservoir acceptability determination. The acceptability determination may be indicated as a failure if any contents of interest are classified as the second content type. In some embodiments, the first content type may be a gas bubble and the second content type is anything other than a gas bubble. In some embodiments, the medical reservoir may be an IV bag.

In accordance with another example embodiment of the present disclosure a method of inspecting a medical reservoir may comprise placing the medical reservoir in a cradle. The method may further comprise providing a background on first side of the medical reservoir which contrasts between light and dark. The method may further comprise illuminating the medical reservoir with an illumination assembly separate from the cradle. The method may further comprise collecting at least one series of frames of the medical reservoir from at least one imager on a second side of the medical reservoir opposite the first. The method may further comprise processing each of the frames in the at least one series of images into a series of processed frames. The method may further comprise detecting regions of interest within each of the processed frames of the series of processed frames. The method may further comprise associating each of the detected regions of interest with tracks. Each track may define the displacement of a specific tracked region of interest over a plurality of processed frames. The method may further comprise analyzing the specific tracked region of interest in each processed frame for each track. The method may further comprise classifying the specific tracked region of interest for each track as one of at least a first content type and a second content type.

In some embodiments, placing the medical reservoir in the cradle may comprise placing an IV bag in the cradle. In some embodiments, the method may further comprise agitating the medical reservoir. In some embodiments, providing the background may comprise placing a pattern of light and black regions on a rest panel of the cradle. In some embodiments, providing the background may comprise adjusting a backlight on the first side of the medical reservoir to provide a background which provides a temporal contrast between light and dark. In some embodiments, illuminating the medical reservoir may comprise directing collimated light at the medical reservoir from an underlight. In some embodiments, illuminating the medial reservoir may comprise directing narrow beam angle light at the medical reservoir from an underlight. In some embodiments, illuminating the medical reservoir may comprise directing polarized light at the medical reservoir. In some embodiments, the at least one imager may include a plurality of imagers each having a field of view which at least partially overlaps that of at least one other imager of the plurality of imagers and collecting the at least one series of frames may comprise collecting a plurality of series of frames. Each plurality of series of frames may be captured from a respective imager of the plurality of imagers. In some embodiments, the method may further comprise providing a polarizing filter for each of the at least one imager. In some embodiments, processing each of the frames may comprise at least one of smoothing each of the frames, generating a foreground segmented image, generating a near edge filtered foreground segmented image based on an edge detection analysis of each frame, performing at least one kernel convolution, performing at least one morphological transformation, and removing pixel clusters having yielding a contour outside of a predefined size range. In some embodiments, detecting regions of interest within each of the processed frames may comprise generating a bounding box around each pixel cluster of interest remaining in the processed frames. In some embodiments, associating each of the detected regions of interest with tracks may comprise determining track predictions for each region of interest in a starting frame and matching the track predictions to detected regions of interest in a subsequent frame. In some embodiments, analyzing the specific tracked region of interest may comprise generating a region of interest score for the specific tracked region of interest in each processed frame of each track. In some embodiments, analyzing the specific tracked region of interest may comprise feeding an extracted portion of each processed frame of each track to a convolutional neural network. Each extracted portion of each processed frame including the specific tracked region of interest. In some embodiments, classifying the specific tracked region of interest for each track as one of at least the first content type and the second content type may further comprise classifying the specific tracked region of interest as at least one content subtype. In some embodiments, the method may further comprise generating an annotated image. In some embodiments, generating the annotated image may comprise depicting a representation of the displacement path of each specific tracked region of interest on a selected frame from each of the at least one series of frames.

In accordance with another example embodiment of the present disclosure, a method of inspecting a medical reservoir may comprise agitating the medical reservoir. The method may further comprise illuminating the medical reservoir with an external illuminator. The method may further comprise illuminating a patterned background on a first side of the reservoir. The method may further comprise capturing a plurality of raw frames of the medical reservoir from a second side of the medical reservoir. The method may further comprise processing, via at least one processor, the plurality of raw frames to create processed frames. The method may further comprise analyzing, via the at least one processor, the processed frames to identify detected pixel clusters of interest. The method may further comprise associating, via the at least one processor, the detected pixel clusters of interest with tracks indicative of the displacement of each pixel cluster of interest over the plurality of frames. The method may further comprise classifying, via the at least one processor, the pixel clusters of interest associated with each track as one of at least a first content type and a second content type.

In some embodiments, agitating the medical reservoir may comprise rotating the medical reservoir through a series of rotational orientations specified in a motion profile. In some embodiments, the method further may comprise generating a log including at least the raw frames and classifications for the pixel clusters of interest associated with each track and communicating the log to an external database. In some embodiments, illuminating the medical reservoir with the external illuminator may comprise directing collimated, polarized light in a preset wavelength range at the medical reservoir. In some embodiments, illuminating the medical reservoir with the external illuminator may comprise directing narrow beam angle, polarized light in a preset wavelength range at the medical reservoir. In some embodiments, illuminating the medical reservoir with the external illuminator may comprise emitting light from a plurality of light emitters at the reservoir. Each light emitter may emit light in a narrow beam angle less than 20°. In some embodiments, illuminating the patterned background may comprise illuminating a rest surface for the medical reservoir. The rest surface may include a checkered pattern of light and dark regions. In some embodiments, capturing the plurality of raw frames may comprise capturing the plurality of raw frames with a plurality of imagers. Each imager may have a field of view which overlaps with the field of view of at least one other imager of the plurality of imagers. In some embodiments, processing the raw frames may comprise at least of generating a foreground mask, detecting edges in the raw frame, generating near edge filtered foreground segmented image from each raw frame, performing at least one morphological transformation, performing at least one kernel convolution, and performing a dilation convolution. In some embodiments, analyzing the processed frames to identify detected pixel clusters of interest may comprise at least one of identifying pixel clusters yielding a contour size outside of a predefined range, identifying pixel clusters which fail at least one predefined validity criteria, performing at least one morphological transformation, performing at least one kernel convolution, performing an erosion convolution, identifying pixel clusters which fail at least one predefined validity criteria, defining bounding boxes around pixel clusters of interest. In some embodiments, processing the raw frames may comprise generating a foreground segmented image from each raw frame, detecting edges in each raw frame and generating an edge image for each raw frame, generating a dilated edge image for each raw frame, generating a near edge filtered foreground segmented image from the foreground segmented image for each raw frame using the respective dilated edge image for each raw frame, and dilating each near edge filtered foreground segmented image. In some embodiments, analyzing the processed frames to identify detected pixel clusters of interest may comprise identifying pixel clusters in each dilated near edge filtered foreground segmented image which fail at least one predefined validity criteria, generated an eroded image from each dilated near edge filtered foreground segmented image in which the identified pixel clusters have been removed, defining bounding boxes around pixel clusters of interest in each eroded image. In some embodiments, associating the detected pixel clusters of interest with tracks may comprise determining track predictions for each detected pixel cluster of interest in a starting frame and matching the track predictions to detected pixel clusters of interest in a subsequent frame. In some embodiments, classifying the pixel clusters of interest associated with each track may comprise generating at least one score for the detected pixel clusters of interest associated with each track. In some embodiments, generating the at least one score may comprise generating at least one of a color score, a trajectory score, a shape score, and a sharpness score. In some embodiments, classifying the pixel clusters of interest associated with each track may comprise classifying the pixel clusters of interest associated with each track with a convolutional neural network trained on images of medical reservoirs with known contents. In some embodiments, classifying the pixel clusters of interest associated with each track may comprise classifying the pixel regions of interest associated with each track as at least one content subtype. In some embodiments, the method may further comprise generating an annotated image. In some embodiments, generating the annotated image may comprise depicting a representation of the displacement path of the pixel cluster of interest associated with each track on a selected frame of the plurality of raw frames. In some embodiments, the method may further comprise generating a pass indication for the medical reservoir when no pixel clusters of interest are classified as the second type and generating a fail indication for the medical reservoir when at least one pixel cluster of interest is classified as the second type. In some embodiments, the method may further comprise generating a troubleshooting suggestion when the fail indication is generated. In some embodiments, the method may further comprise detecting a pattern indicative of the patterned background being distorted by an air bubble in one of the plurality of raw frames and processed frames. In some embodiments, the medical reservoir may be a bag.

In accordance with another example embodiment of the present disclosure a particulate inspection system for detecting and classifying contents of interest within a medical reservoir may comprise a reservoir rest body having a pattern of light and dark regions. The system may further comprise a rest body backlight. The system may further comprise an external illuminator. The system may further comprise a vision assembly opposite the rest body. The vision assembly may include at least one imager. Each imager may have a field of view encompassing at least a portion of rest body. The system may further comprise a controller in data communication with each of the at least one imager. The controller may be configured to command capture of a series of images from each of the at least one imager, receive the images, process the images into processed images, detect regions of interest within the processed images, analyze the regions of interest, and classify the regions of interest as one of a bubble and something other than bubble.

In some embodiments, the system may further comprise a rotary actuator coupled to the rest body. In some embodiments, the rest body backlight may emit white light. In some embodiments, the pattern may be a checkered pattern. In some embodiments, the system may further comprises a reservoir retainer container coupled to reservoir rest body. The reservoir retainer may include a set of IV bag port graspers actuatable between an open state and port retaining state. In some embodiments, the external illuminator may be an underlight. In some embodiments, the external illuminator may include a plurality of LEDs electrically coupled in series. In some embodiments, the external illuminator may include a plurality collimated light emitters with a narrow beam angle less than 20°. In some embodiments, the external illuminator may include a polarizer. In some embodiments, each of the at least one imager may include a polarizing filter on a lens thereof. In some embodiments, the vision system may includes a plurality of imagers. Each field of view of each imager may overlap the field of view of at least one other imager of the vision system. In some embodiments, the view axis each of the at least one imager is oriented at an angle 30-35° from a plane extending perpendicular to the height axis of the rest body. In some embodiments, the controller may be further configured to assign each region of interest in each of the processed images to one or more track. Each of the one or more track may define the displacement of an associated region of interest throughout the plurality of processed images. In some embodiments, for each track, the controller may be configured to extract an extracted portion of every processed image in which the associated region of interest is detected. The extracted portion may include the associated region of interest. The controller may be configured to analyze the regions of interest by analyzing the extracted portions. In some embodiments, the controller may be configured to analyze the regions of interest with a convolutional neural network. In some embodiments, the controller may be configured generate a reservoir acceptability determination. The acceptability determination may be indicated as a failure if any contents of interest are classified as something other than a bubble. In some embodiments, the medical reservoir may be an IV bag.

In accordance with another example embodiment of the present disclosure a method of capturing images of a medical reservoir for inspection may comprise positioning a first side of the medical reservoir against a reservoir rest. The method may further comprise providing, with a first illuminator, a contrasting background for the medical reservoir. The method may further comprise illuminating the medical reservoir with a second illuminator which emits narrow beam angle, polarized light in at least one predefined wavelength range. The method may further comprise capturing a plurality of images of the medical reservoir with at least one imager on a second side of the medical reservoir opposite the first side. Each of the at least one imager being associated with a polarizing filter.

In some embodiments, positioning the first side of the medical reservoir against the reservoir rest may comprise retaining the medical reservoir with a retainer assembly. In some embodiments, the medical reservoir may be an IV bag and retaining the medical reservoir may comprise retaining one or more port of the IV bag in one of a passive retainer and a set of grasper jaws. In some embodiments, the method may further comprise agitating the medical reservoir. In some embodiments, agitating the medical reservoir may comprise rotating the medical reservoir between at least one pair of predefined rotational orientations. In some embodiments, the method may further comprise waiting a dwell period after agitating the medical reservoir before capturing the plurality of images. In some embodiments, providing the contrasting background may comprise backlighting the reservoir rest. The reservoir rest may include a pattern of light and dark regions. In some embodiments, the pattern of light and dark regions may be a checker pattern. In some embodiments, providing the contrasting background may comprise adjusting the output of the first illuminator over time. In some embodiments, illuminating the medical reservoir with a second illuminator may comprise under lighting the medical reservoir with the second illuminator. In some embodiments, one of the at least one predefined wavelength range may be selected from a list consisting of 580-620 nm and 620-750 nm. In some embodiments, the at least one imager may include a plurality of imagers. A field of view of each of the plurality of imagers may overlap the field of view at least one other imager of the plurality of imagers. In some embodiments, the field of view of each of the plurality of imagers may overlap the field of view at least one other imager of the plurality of imagers by at least 10%. In some embodiments, each of the plurality of imagers may be oriented with a view axis at an angle 30-35° from a plane extending perpendicular to the height axis of the reservoir rest. In some embodiments, capturing a plurality of images of the medical reservoir with at least one imager on a second side of the medical reservoir may comprise capturing the plurality of images at a frame rate of at least 8 frames per second from each of the at least one imager.

In accordance with another example embodiment of the present disclosure a method identifying an object in an image may comprise detecting the object in an image. The method may further comprise analyzing the object. The method may further comprise, if analyzing reveals that the object is constructed of a first material type, categorizing the object as an undesired content type. The method may further comprise, if said analyzing reveals that the object has a dimension exceeding a threshold or is constructed of a second material type, conducting a second analysis the object. The method may further comprise categorizing the object a benign content type or the undesired content type depending on a classification output by the second analysis.

In some embodiments, the threshold may be 180 microns. In some embodiments, the first material type may be materials which are opaque and substantially absorb visible light. In some embodiments, the second material type may be materials that are transmissive and materials that are reflective. In some embodiments, detecting the object may comprise performing background subtraction using the image and a reference image. In some embodiments, conducting the second analysis may comprise supplying at least a portion of the image to a trained neural network. In some embodiments, the trained neural network may be a convolutional neural network. In some embodiments, the method may further comprise projecting a light from at least one first light source in a direction substantially orthogonal to a view axis of an imager capturing the image. In some embodiments, the light from the at least one first light source may be selected from a list consisting of collimate or narrow beam angle. In some embodiments, the method may further comprise orienting a reservoir such that a view axis of an imager capturing the images passes through a portion of the reservoir. In some embodiments, the reservoir may be an IV bag. In some embodiments, the method may further comprise filtering the image. In some embodiments, the method may further comprise positioning a reservoir intermediate a background and an imager capturing the image. In some embodiments, the method may further comprise illuminating the background. In some embodiments, illuminating the background may comprise backlighting the background. In some embodiments, the background may include a checkered pattern.

In accordance with another example embodiment of the present disclosure a method of identifying a content within a reservoir may comprise projecting a light at the reservoir. The method may further comprise capturing an image of the reservoir with an imager. The light may be selected from a list consisting of collimated and a narrow beam angle projected light from at least one light source. The light may be substantially projected orthogonal to a view axis of the imager.

In some embodiments, the reservoir may be an IV bag. In some embodiments, the method may further comprise filtering the image. In some embodiments, the method may further comprise disposing the reservoir intermediate a background pattern and the imager. In some embodiments, the background may be illuminated. In some embodiments, the background may be backlit.

In accordance with another example embodiment of the present disclosure a system for identifying a content of interest in a reservoir may comprise an imager having a view axis and a view field. The system may further comprise a light source. The light source may selected from a list consisting of a collimated light source and a narrow beam angle light source. The light source may project light in a direction substantially orthogonal to the view axis and the view field.

In some embodiments, the system may further comprise a cradle shaped to accept the reservoir. In some embodiments, the system may further comprise a background opposite the imager. In some embodiments, the system may further comprise a background illuminator for illuminating the background. In some embodiments, the background illuminator may be a backlight. In some embodiments, the background may include a checkered pattern. In some embodiments, the system may further comprise a frame configured to retain the reservoir intermediate the background and imager and position the reservoir in an illumination field of the light source.

In accordance with another example embodiment of the present disclosure a method of verifying hardware functionality of a particulate inspection system for inspecting reservoirs may comprise capturing a first test image of a portion of the particulate inspection system in a first condition. The method may further comprise performing a first comparison, with a controller, of the first test image with a first reference image and generating a first fault notification with the controller when the first comparison fails a first quality criteria. The method may further comprise capturing a second test image of the portion of the particulate inspection system in a second condition when the first comparison passes the first quality criteria. The method may further comprise performing a second comparison, with the controller, of the second test image with a second reference image and generating a second fault notification with the controller when the second comparison fails a second quality criteria. The method may further comprise capturing a third test image of the portion of the particulate inspection system in a third condition when the second comparison passes the second quality criteria. The method may further comprise performing a third comparison, with the controller, of the third test image with a third reference image and generating a third fault notification with the controller when the third comparison fails a third quality criteria. The method may further comprise populating an inspection log for the reservoir with results of the first, second, and third comparison.

In some embodiments, the first condition may be a condition in which a back light and external illuminator of the particulate inspection system are unpowered. In some embodiments, the second condition may be a condition in which a back light of the particulate inspection system is powered and the portion of the particulate inspection system may be a patterned rest body which is illuminated by the back light. In some embodiments, the third condition may be a condition in which an external illuminator of the particulate inspection system is powered. In some embodiments, performing the first comparison may comprise aligning the first test image with the first reference image. In some embodiments, performing the first comparison may comprise performing a mean square error analysis with the first test image and first reference image. In some embodiments, the first quality criteria may be a mean square error threshold above which the first comparison is failed. In some embodiments, performing the second comparison may comprise aligning the second test image with the second reference image. In some embodiments, performing the second comparison may comprise performing a mean square error analysis with the second test image and second reference image. In some embodiments, the second quality criteria may be a mean square error threshold above which the second comparison is failed. In some embodiments, performing the third comparison may comprise aligning the third test image with the third reference image. In some embodiments, performing the third comparison may comprise performing a mean square error analysis with the third test image and third reference image. In some embodiments, the third quality criteria may be a mean square error threshold above which the third comparison is failed. In some embodiments, the method may further comprise installing the reservoir in the particulate inspection system and capturing a fourth test image of the portion of the particulate inspection system. In some embodiments, the method may further comprise performing a fourth comparison of the fourth test image to a fourth reference image and generating a notification, with a controller, when the fourth comparison fails a fourth quality criteria. In some embodiments, the fourth reference image may be of the portion of the particulate inspection system with no reservoir installed in the particulate inspection system. In some embodiments, performing the fourth comparison may comprise aligning the fourth test image against the fourth reference image and performing a mean square error analysis. The fourth quality criteria may be a mean square error threshold below which the fourth quality criteria is failed.

### BRIEF DESCRIPTION OF THE DRAWINGS

These and other aspects will become more apparent from the following detailed description of the various embodiments of the present disclosure with reference to the drawings wherein:

FIGS. 1A-12B depicts various example mix assisting assemblies;

FIG. 13A depicts a rear perspective view of the example mix assisting assembly of FIG. 12A with a rear panel removed;

FIG. 13B depicts a detailed view of the indicated region of FIG. 13A;

FIG. 14A depicts a view of an example mix assisting assembly including doors which are in an open state;

FIG. 14B depicts a rear view of the example mix assisting assembly of FIG. 14A;

FIG. 15A depicts a filled bag in place within an example mix assisting assembly;

FIG. 15B depicts a rear view of the example mix assisting assembly of FIG. 14A with the doors in a closed state;

FIG. 16 depicts a perspective view of an example door which may be included in a mix assisting assembly;

FIG. 17 depicts a flowchart detailing a number of example actions which may be executed to mix contents of a reservoir with a mix assisting assembly of the type shown in FIG. 14A;

FIG. 18 depicts a block diagram of an example particulate inspection system;

FIG. 19A depicts a perspective view of an example particulate inspection system;

FIG. 19B depicts a perspective view of an example particulate inspection system with a bag type reservoir installed therein;

FIG. 19C depicts a cross-sectional view of an example particulate inspection system;

FIG. 20A depicts a perspective view of an example external illuminator which may be included in an example particulate inspection system;

FIG. 20B depicts a cross-sectional view of an example external illuminator which may be included in an example particulate inspection system;

FIG. 20C depicts a detailed view of the indicated region of FIG. 20B;

FIG. 21 depicts a flowchart detailing a number of example actions which may be executed to conducted a functionality check for an example particulate inspection system;

FIG. 22A depicts a representation of an image of a reservoir in a particulate inspection system;

FIG. 22B depicts an enlarged view of a portion of FIG. 22A;

FIGS. 23A-B depict diagrams of collimated light passing through a bubble;

FIGS. 24A-D depict a progression of image representations of a reservoir in which a bubble is displacing over a period of time;

FIG. 25 is a representation of an image of a reservoir in place in a particulate inspection system;

FIG. 26 depicts a representation of an image of a reservoir having a number of various pieces of particulate therein;

FIG. 27A-D depict a progression of image representations in which a piece of particulate is displacing within a reservoir of time;

FIG. 28 depicts a data flow diagram for a particulate inspection system;

FIG. 29 depicts a flowchart detailing a number of example actions which may be executed to inspect a reservoir with a particulate inspection system and route the reservoir through a system based on the inspection result;

FIG. 30 depicts a flowchart detailing a number of example actions which may be executed to detect, track, and classify regions of interest within a reservoir and generate a pass/fail determination for the reservoir;

FIG. 31 depicts a flowchart detailing a number of example actions which may be executed to detect regions of interest within a reservoir;

FIG. 32 depicts a representation of a raw image of a reservoir in place at a particulate inspection system;

FIG. 33A depicts a representation of a foreground segmented image of a reservoir in place in a particulate inspection system;

FIGS. 33B-C depict enlarged views of the indicated regions in FIG. 33A;

FIG. 34 depicts a representation of an example image of edge detections after a morphological transformation;

FIG. 35A depicts a representation of an near edge filtered foreground segmented image;

FIGS. 35B-C depict enlarged views of the indicated regions in FIG. 35A;

FIG. 36 depicts a representation of the image of FIG. 35A after a morphological transformation;

FIG. 37 depicts a representation of the image of FIG. 36 with pixel clusters having an area greater than a predefined threshold removed;

FIG. 38A depicts a representation of the image of FIG. 37 after a morphological transformation;

FIG. 38B-C depict enlarged views of the indicated regions in FIG. 38A;

FIG. 39A depicts a representation of the image of FIG. 32 with bounding boxes applied around detected regions of interest;

FIG. 39B depicts an enlarged view of the indicated region of FIG. 39A;

FIG. 40A depicts a representation of an image of a reservoir in place at a particulate inspection systems taken some period of time after the image of FIG. 32 with bounding boxes applied around detected regions of interest;

FIG. 40B depicts an enlarged view of the indicated region of FIG. 40A;

FIG. 41 depicts a flowchart detailing a number of example actions which may be executed to track regions of interest over a series of frames of a reservoir in place in a particulate inspection system;

FIG. 42A-D depict a track for a region of interest as it grows over a series of frames;

FIG. 43 depicts the track of FIGS. 42A-D overlaid on a representation of a portion of an image of a reservoir against a background pattern;

FIG. 44 depicts a flowchart detailing a number of example actions which may be executed to score and classify regions of interest detected in a reservoir in place at a particulate inspection system;

FIG. 45A depicts an example annotated image of a reservoir in place at a particulate inspection system showing a number of tracks for detected regions of interest;

FIG. 45B depicts an example annotated image of a reservoir in place at a particulate inspection system showing a number of tracks for detected regions of interest;

FIGS. 46-48 are views of a portion of an example embodiment of a system for identifying an object;

FIG. 49 is a front view of a portion of an example embodiment of a system for identifying an object depicting a reservoir being illuminated;

FIG. 50 is a perspective view of a portion of an example embodiment of a system for identifying an object depicting fields of view of example imagers in the system;

FIG. 51 is a perspective view of an example system for identifying an object;

FIGS. 52A-B depict Fresnel reflectance and transmittance plots over angle of incidence;

FIG. 53 is a representation of an image of a reservoir against a background;

FIG. 54A is a representation of an image of a first content type within a reservoir;

FIG. 54B is a representation of an image of a second content type within a reservoir;

FIG. 54C is a representation of an image of a third content type within a reservoir;

FIG. 54D is a representation of an image of a fourth content type within a reservoir;

FIG. 54E is a representation of an image of a fifth content type within a reservoir;

FIG. 54F is a representation of an image of a sixth content type within a reservoir;

FIGS. 55A-B depict an illustrative representation of a simulated image of an illuminated opaque white particle against a chessboard patterned background;

FIGS. 56A-B depict an illustrative representation of a simulated image of an illuminated opaque reflective particle against a chessboard patterned background;

FIGS. 57A-B depict an illustrative representation of a simulated image of an illuminated particle against a chessboard patterned background, the particle being a material which is opaque and dark in color;

FIGS. 58A-B depict an illustrative representation of a simulated image of an illuminated transparent particle against a chessboard patterned background;

FIG. 59A depicts an illustrative representation of a simulated image of an illuminated bubble in a reservoir;

FIG. 59B. depicts an illustrative representation of a simulated image of an illuminated bubble in a reservoir;

FIG. 60A is a representation of an image of a bubble within a reservoir placed against a patterned background, the bubble being illuminated with a narrow beam light source from below;

FIG. 60B is a representation of an image of a bubble after being expelled from a needle into a reservoir, the reservoir being positioned against a patterned background, the bubble being illuminated with a narrow beam light source from below;

FIG. 61 is an example diagram of collimated light being emitted at a bubble;

FIG. 62 depicts a diagram of an example bubble being illuminated with collimated light;

FIG. 63 depicts a diagram of an example bubble being illuminated with collimated light;

FIG. 64 depicts example bubbles being illuminated;

FIG. 65 depicts a diagram of an example bubble being illuminated;

FIG. 66A depicts an illustrative representation of a simulated image of an illuminated bubble in a reservoir;

FIG. 66B depicts an illustrative representation of a simulated image of an illuminated bubble in a reservoir;

FIG. 67A depicts an illustrative representation of a simulated image of an illuminated bubble in a reservoir;

FIG. 67B depicts an illustrative representation of a simulated image of an illuminated bubble in a reservoir;

FIG. 68 depicts a flowchart detailing a number of example actions which may be executed to identify and categorize objects in a reservoir;

FIG. 69 depicts a flowchart detailing a number of example actions which may be executed to identify and categorize objects in a reservoir; and

FIG. 70 depicts an example data flow diagram for an example particulate inspection system.

### DETAILED DESCRIPTION

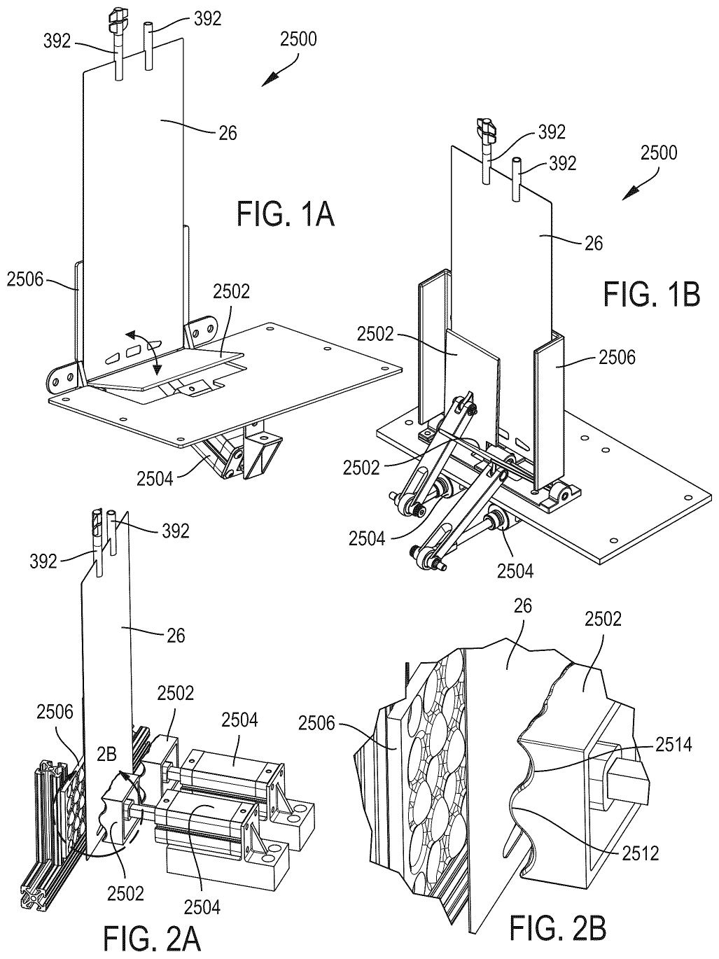

Referring to FIGS. 1-3, a number of exemplary mix assisting assemblies 2500 and bags 26 are depicted. Mix assisting assemblies 2500 may, for example, be used to mix fluid in bags 26 which are supplied with a concentrate and subsequently filled to a desired capacity. The concentrate may be a solid (e.g. powder, crystalline concentrate, lyophilized medicament, etc.) or liquid (e.g. concentrated medical solution, brine, etc.). In certain implementations, a mix assisting assembly 2500 may also be utilized with a bag 26 having a plurality of internal chambers which are user or machine interruptible, though use with bags 26 having a single, continuous interior volume is also possible. Example mix assisting assemblies 2500 may act upon an exterior of a bag 26 to encourage mixing the contents of the bag 26. Alternatively or additionally, mix assisting assemblies 2500 may also displace an entire bag 26 to encourage mixing of the contents of the bag 26. In certain embodiments, an example mix assisting assembly 2500 may be operated until a solution formed in a bag 26 has substantially uniform characteristics throughout the bag 26. Mix assisting assemblies 2500 may, for example, mix concentrate with fluid, excipient, or diluent (e.g. Water for Injection) dispensed into a bag 26. In embodiments where the concentrate is a solid (e.g. crystalline salt) the mix assisting assembly 2500 may also encourage dissolution of the concentrate into fluid dispensed into the bag 26. A mix assisting assembly 2500 may further cause any particulate within a bag 26 to displace within the bag 26. Thus, the mix assisting assembly 2500 may also be a particulate identification assist assembly where bags 26 are checked for particulate via a machine vision system. A mix assisting assembly 2500 may be included in any location within a fluid production and packaging system, however, in certain examples, a mix assisting assembly 2500 may be included as a part of a filling station at which fluid is dispensed into the bag 26. Alternatively, a mix assisting assembly 2500 may be a separate station which a bag 26 may be passed to after a fill station. This may allow a bag 26 to be mixed while another bag 26 is being filled at a given fill station.

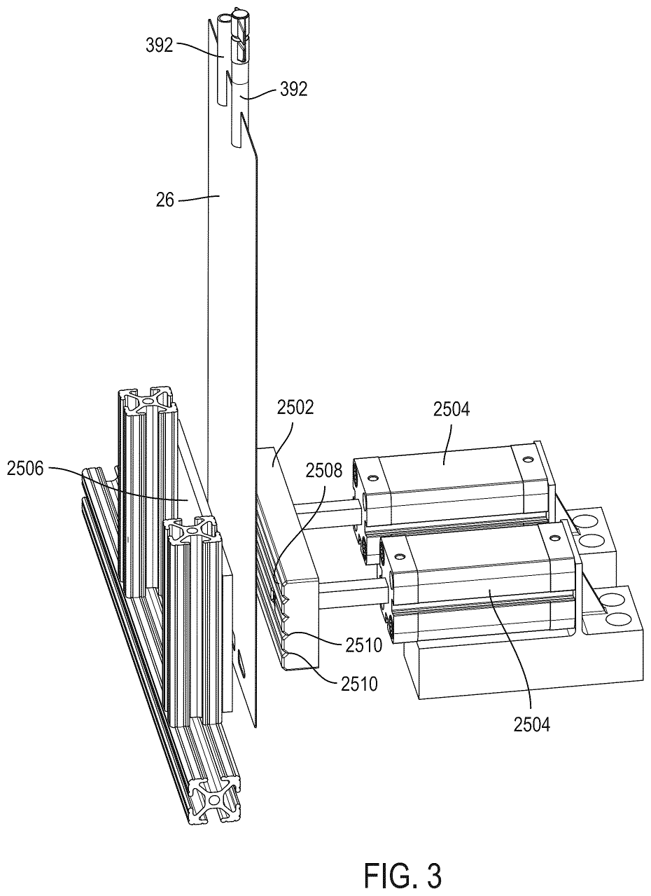

Certain mix assisting assemblies 2500 may include at least one displaceable body 2502 which may be driven against and away from the exterior of a bag 26 to displace fluid within the bag 26. At least a portion of a bag 26 may rest against a plate 2506 (e.g. back plate) as the mix assisting assembly 2500 is operated to mix fluid within the bag 26. The mix assisting assembly 2500 may include one or more actuator 2504. Each of the at least one displaceable body 2502 may be coupled to an actuator 2504 such that the actuator 2504 may be powered by the control system 15 to drive displacement of that displaceable body 2502. Displaceable bodies 2502 may be displaced against the bag 26 along a variety of displacement paths. For example, the displaceable bodies 2502 may be pivoted about a rotation axis into the bag 26 (see, e.g., FIG. 1A-B). In such embodiments, a displaceable body 2502 may follow a curved or arcuate pathway as it is displaced toward and away from a bag 26. Alternatively (see, e.g. FIGS. 2A-3), displaceable bodies 2502 may be displaced along a substantially straight displacement path (e.g. perpendicular to side seams of the bag 26). In some examples, a single displaceable body 2502 may be driven toward and away from the bag 26 by one (see, e.g., FIG. 1A) or more (see, e.g., FIG. 3) actuators 2504. In some examples, multiple displaceable bodies 2502 (see, e.g., FIG. 2A) may be displaced against the bag 26 in some synchronized relationship governed by the control system 15. For example, one displaceable body 2502 may be displaced with respect to a bag 26 out of phase with a second displaceable body 2502 by a predefined amount. One displaceable body 2502 may be displaced against a bag 26 while the other is retracted and vice versa, thus the displaceable bodies 2502 may be displaced 180° out of phase with one another. Any other phase relationship or a varying relationship may be used.

Displaceable bodies 2502 may be displaced against any desired portion or portions of a bag 26. In the example embodiments, the displaceable bodies 2502 are displaceable toward and away from a portion of the bags 26 most distal to the ports 392 (the bottom of the bags 26 with respect to pull of gravity in FIGS. 1A-3). Where multiple displaceable bodies 2502 are displaced with respect to a bag 26, they may be displaced against the same region (e.g. bottom) of the bag 26 or one may be displaced against a first region of the bag 26 and another may be displaced against a second region of the bag 26.

Displaceable bodies 2502 may include a contact face 2508 which may press against the exterior of a bag 26 during operation. The contact face 2508 may include one or more raised and/or recessed feature which may help to encourage or direct mixing of contents in a bag 26. The contact face 2508 may include an arrangement of channels 2510 like those shown in FIG. 3. In other embodiments, the contact face 2508 may include a set of raised and recessed features. Such features may be disposed in a repeating pattern. In some examples, the contact faces 2508 may include raised and recessed features in an egg crate type pattern. As best shown in the example in FIG. 2B, raised convex features 2512 and recessed concave features 2514 may be defined on the contact face 2508. In various examples, and as shown in FIG. 2B, the plate 2506 against which the bag 26 rests may also include one or more raised or recess feature. The features of the plate 2506 and contact face 2508 may be arranged offset to one another so as to interdigitate or intermesh with one another when a displaceable body 2502 reaches an end of its displacement range.

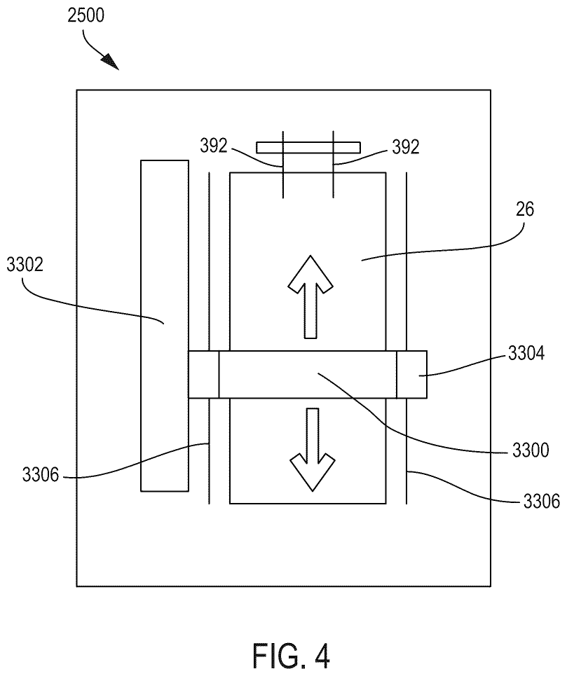

Referring now to FIG. 4, another example mix assisting assembly 2500 is depicted. Certain mix assisting assemblies 2500 may be displaced against a bag 26 and may be driven to adjust an area of the bag 26 which is locally depressed. In such examples, the mix assisting assembly 2500 may include at least one roller 3300. Each of the at least one roller 3300 may be attached to a roller actuator 3302 which may be powered to displace the roller(s) 3300 along the bag 26. The roller(s) 3300 may be coupled to a mount 3304 which may displace along a set of tracks 3306 included in the mix assisting assembly 2500. In some instances, the roller(s) 3300 may be spring biased against the bag 26. For example, a roller 3300 may be coupled to a trunnion which is spring biased towards the bag 26. The trunnion may displaced along guides included in the mount 3304 under the bias of the springs. In the example embodiment, a single roller 3300 is included.

The roller 3300 may be driven along the length of the bag 26 under the direction of control system 15 commands issued to the roller actuator 3302. In certain examples, the roller 3300 may be disposed at a starting position near the ports 392 when a bag 26 initially begins to be filled. The roller 3300 may press against the bag 26 so as to prohibit flow of fluid past the roller 3300 as the bag 26 is filled. As fluid is dispensed into the bag 26, the roller 3300 may be displaced to one or more additional position against the bag 26. The roller 3300 may be displaced quickly and in stepwise manner to the additional positions. This may cause fluid initially filled into the bag 26 to turbulently drop encouraging mixing as the roller 3300 is relocated. Alternatively, the roller 3300 may be displaced in continuous manner as the bag 26 is filled with fluid. This may ensure any concentrate in the bag 26 is kept in motion and does not take on a substantially stationary position at the end of the bag 26 opposite the ports 392 shortly after filling begins. In some examples, the roller 3300 may be displaced proximal and distal the ports 392 to promote mixing as the bag 26 is filled. Alternatively, the roller 3300 may be positioned against the bag 26 at a position opposite the ports 392 and displaced toward the ports 392 as the bag 26 is filled. In some examples, a fraction of the intended fill volume may be delivered to the bag 26 and filling of the bag 26 may be paused. The roller 3300 may be actuated to displace fluid within the bag 26 (in any of the above described manners) to assist in creating a homogenous solution in the bag 26. Filling may subsequently resume. Filling may be paused multiple times as a bag 26 is filled to mix fluid within the bag 26 via a mix assisting assembly 2500 (any of those described herein).

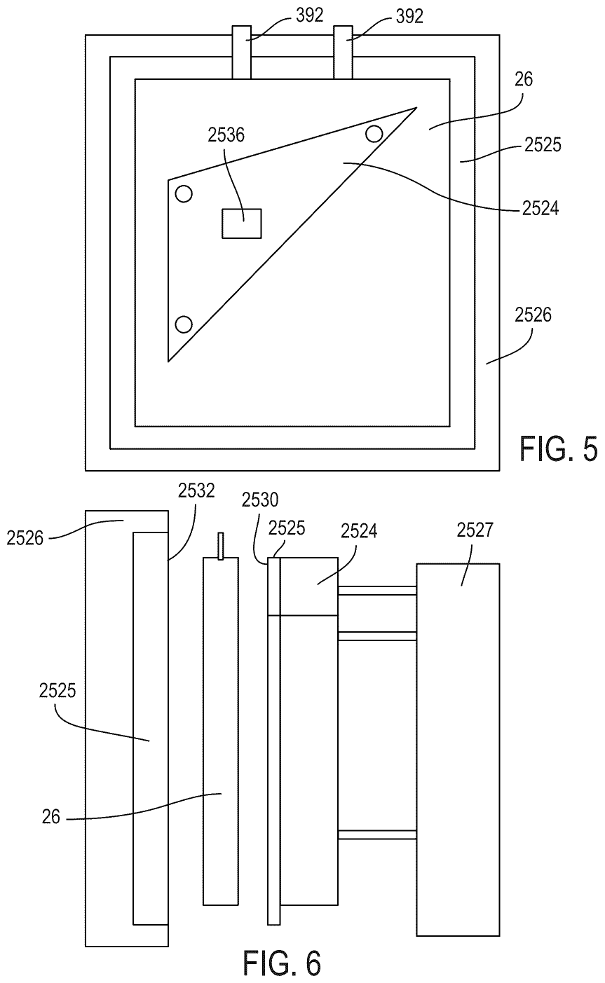

Referring now to FIG. 5-6, another exemplary mix assisting assembly 2500 is depicted. In certain embodiments, select regions of a bag 26 may be blocked off as a bag 26 is filled with fluid from a dispensing sharp or other dispensing outlet. For example, desired regions of a bag 26 may be clamped or pressed closed via the exterior of the bag 26 to define a desired flow path for incoming fluid as the bag 26 is filled. The desired flow path may be a temporary flow path which is imposed upon a bag 26 until the bag 26 has been filled with at least some volume of fluid. The desired flow path may be a tortuous flow path which is defined to engender turbulent flow and bolster mixing within the bag 26 as fluid travels along the temporary flow path. Alternatively or additionally, at least a portion of the imposed flow path may create a small passage within the bag 26 through which fluid may flow. This may cause the velocity of the fluid to increase in any such areas. The high velocity fluid may be directed to a region of the bag 26 to agitate fluid within the bag 26. The above described externally imposed flow paths may aid in dissolving and mixing any concentrate within the bag 26, and help ensure a uniform solution is formed as a bag 26 is filled with fluid. At least a portion of the bag 26 may be unclamped as the fill level of the bag 26 increases and any temporary flow path in that portion of the bag 26 may be removed. The bag 26 may be completely unclamped and the temporary flow path may be completely removed when the bag 26 is full or nearly full. Data from a flow sensor in communication with the control system 15 may be monitored to determine when certain fill thresholds have been met. The control system 15 may send commands to unclamp the bag 26 when certain fill threshold criteria is/are satisfied.

As shown in the block diagram depicted in FIG. 5-6, a bag 26 may be displaced to a fill station. The fill station may include a first plate 2524 and opposing second plate 2526. At least one of the plates 2524, 2526 may be displaceable between a deployed and a retracted position. The bag 26 may be disposed between the first and second plates 2524, 2526 and at least one of the plates 2524, 2526 may be displaced to capture or sandwich the bag 26 between the plates 2524, 2526 during filling. Each plate 2524, 2526 may include a contact face 2530, 2532 which may at least partially abut a bag 26 as the bag 26 is filled. Contact face 2530, 2532 described herein may include at least one compliant member 2525 (e.g. gasket, overmolded compliant material, etc.). With the bag 26 captured between the plates 2524, 2526, the walls of the bag 26 defining the interior volume of the bag 26 may be pressed or clamped together in regions between the contact faces 2530, 2532. These regions may be substantially sealed or blocked off to fluid flow as fluid is transferred into the bag 26. The first and second plate 2524, 2526 may be held together in any suitable manner. In some embodiments, at least one actuator may be included to drive at least one of the first and second plate 2524, 2526 toward the other. In alternative embodiments, and as shown, the plates 2524, 2526 may be held together magnetically with one or more magnet 2536. In some embodiments, the plates 2524, 2526 may clamp the bag 26 via magnetic attraction and may be forced apart as sufficient fluid has been dispensed into the bag 26. At least one of the plates 2524, 2526 may be on a guide assembly 2527 (partially hidden in FIG. 5) which directs motion of the plate 2524, 2526 toward and away from the bag 26.

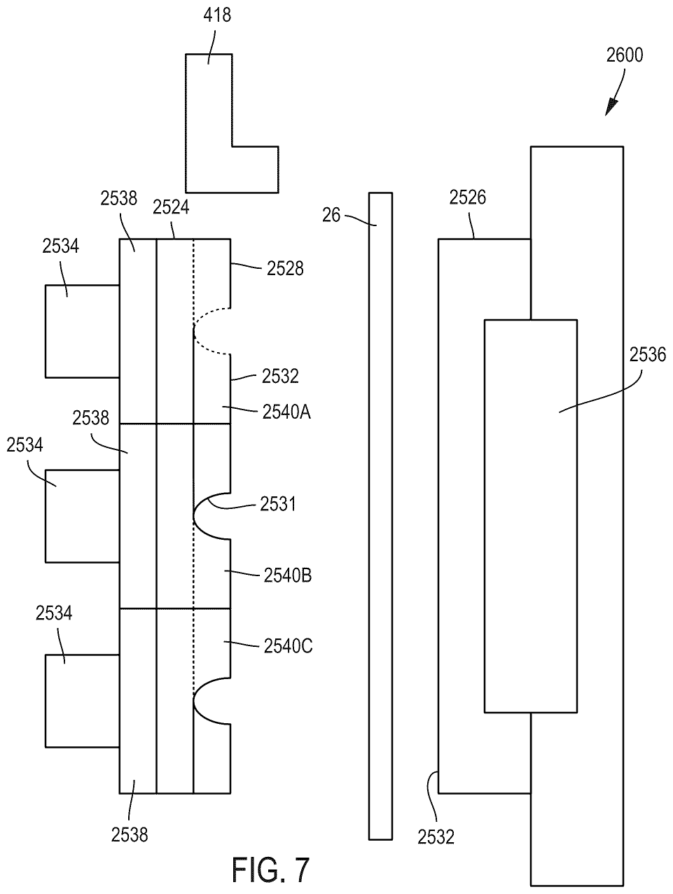

Referring now primarily to FIG. 7, in some embodiments, unclamping of a bag 26 as the bag 26 is filled may occur in stages. For example, a first portion of the bag 26 may be unclamped once a first amount of fluid has been filled into the bag 26. A second portion of the bag 26 may be unclamped once a second amount of fluid has been filled into the bag 26. Any number of additional portions of the bag 26 may be unclamped as the fill level of the bag 26 reaches thresholds defined for each respective portion. Commands may be sent from the control system 15 to unclamp appropriate regions of the bag 26 as respective fill thresholds are met. In such embodiments, one of the plates 2524, 2526 may be divided into a number of units 2540A-C which may be displaceable in tandem as well as independent of one another.

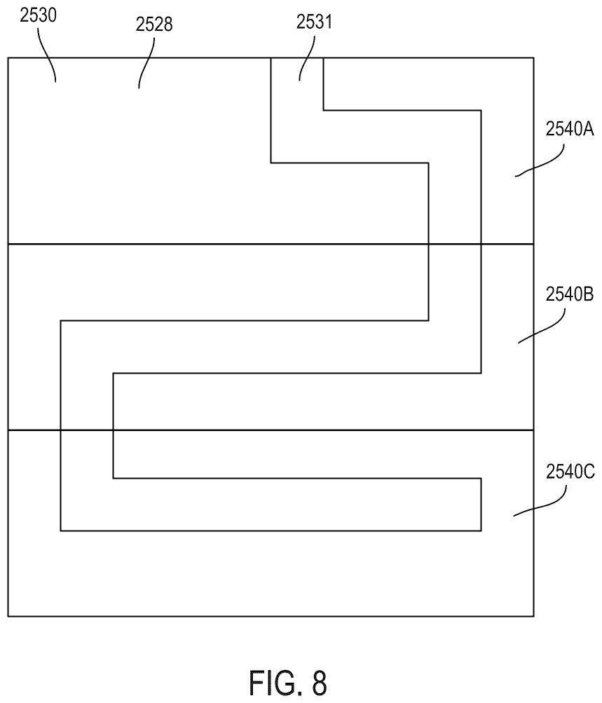

Additionally, in certain examples, the contact face 2530, 2532 of at least one of the plates 2524, 2526 may include a raised face 2528 and at least one recessed portion 2531. Fluid introduced to the bag 26 may travel along a flow path through the bag 26 in regions of the bag 26 present between the recessed portion 2531 and the opposing contact face 2532. Thus, a desired temporary flow path may be imposed upon a bag 26 from the exterior of the bag 26. A view of an example contact face 2530 is depicted it FIG. 8. The layout of the recessed portion 2531 is arranged to impose a flow path on the bag 26 which meanders back and forth across the width of the bag 26. Any desired flow path may be imposed by adjusting the layout of the recessed portion 2531. The recessed portion 2531 has a constant width, though could alter in width at at least one region to adjust the flow velocity within the imposed flow path.

Referring to both FIG. 7-8, the plates 2524, 2526 may be held together magnetically. The fill station 2600 may include at least one retention magnet 2536 and at least one retraction magnet 2534. The retention magnets 2536 may be associated with the second plate 2526. The retraction magnets 2534 may be disposed adjacent a retracted position of the first plate 2524. The first plate 2524 may include at least one metallic body 2538 which may be attracted by the retention and retraction magnets 2534, 2536. A gripper 418, which may be included on a gantry or robotic arm, may transfer a bag 26 to the fill station. The gripper 418 may also collect the first plate 2524 and disengage the first plate 2524 from the at least one retraction magnet 2534. The first plate 2524 or each unit 2540A-C of the first plate 2524 may be displaced (e.g. along one or more guide of a guide assembly 2527) by the gripper 418 toward the second plate 2526. The retention magnets 2536 may magnetically attract and hold the first plate 2524 or units 2540A-C thereof against the second plate 2526 capturing a bag 26 between the plates 2524, 2526. This position of the first plate 2524 may be referred to as a deployed position. As the bag 26 is filled, the fluid filled into the bag 26 may eventually push the first plate 2524 away from the second plate 2526 and toward the retracted position as the bag 26 increases in volume. The first plate 2524 (or units 2540A-C thereof) may be displaced by the expanding bag 26 a distance sufficient for the attraction of the retraction magnet(s) 2534 to pull the first plate 2524 to the retracted position.

As mentioned above, the first plate 2524 may be divided into a set of individually displaceable units 2540A-C. Though three are shown, any number of individually displaceable units 2540A-C is possible in other embodiment. Each of the units 2540A-C may be disengaged from the at least one retention magnet(s) 2536 and moved to a retracted position as the bag 26 reaches a respective fill volume. Thus the temporary flow path imposed by the plates 2524, 2526 may be removed in stages. In certain examples, the displaceable units 2540A-C may be retracted in order of their proximity to the ports 392 of the bag 26. The displaceable unit 2540A-C most proximal the ports 392 may be retracted first and the displaceable unit 2540A-C most distal the ports 392 being retracted last.

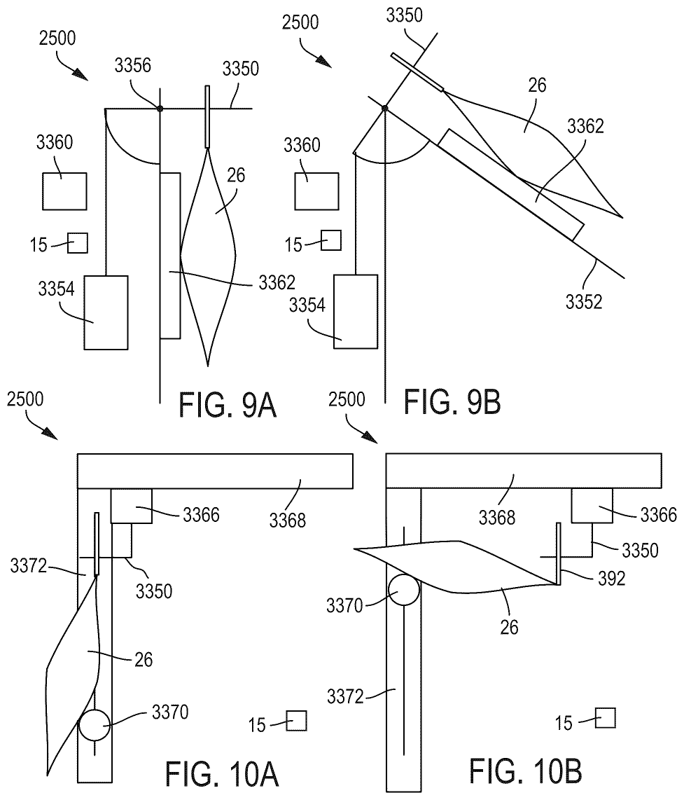

In some examples, the bag 26 may be manipulated in one or more additional manner while a mix assisting assembly 2500 imposes the flow path on the bag 26. For example, the bag 26 may be displaced such that the end of the bag 26 distal to the ports 392 is disposed above the ports 392 (see, e.g. FIGS. 9A-11C). Alternatively or additionally, one or more displaceable body 2502 may be displaced against and away from the bag 26.

Certain example mix assisting assemblies 2500 may displace the entire bag 26 in order to encourage generation of a homogenous solution within the bag 26. The bag 26 may for example be rotated about an axis by the mix assisting assembly 2500 to engender movement of the fluid within the bag 26. This movement may generate eddies which may persist after the bag 26 has been rendered stationary helping to thoroughly mix contents of the bag 26.

An example mix assisting assembly 2500 which displaces a bag 26 to encourage mixing is depicted in FIGS. 9A-B. As shown, the mix assisting assembly 2500 may include a bag retainer 3350. The ports 392 of the bag 26 may be placed into the bag retainer 3350 to hold the bag 26 in place. The bag 26 may rest against a support 3352 when in the bag retainer 3350. The bag retainer 3350 may be disposed on the support 3352 in certain examples. The support 3352 may be coupled to a pivot actuator 3354 and may be pivotally displaceable about a pivot 3356. The pivot actuator 3354 may be powered to displace the support 3352 about the axis of the pivot 3356. In the example, the axis of the pivot 3356 is generally parallel to the width dimension of the bag 26 and outside of the footprint of the bag 26. Fluid in the bag 26 may be encouraged to mix by displacing the support 3352 relative to a back plate 3358 of the mix assisting assembly 2500. The support 3352 may, for example, be pivoted toward and away from the back plate 3358 a predefined number of times by the pivot actuator 3354 to mix fluid within the bag 26. The control system 15 may displace the support 3352 to preset angular positions (e.g. based on feedback from a support position sensor 3360). Each displacement away from the back plate 3358 may be to a respective preset angle. At least one of the preset angles may be greater than 90°. Each displacement toward the back plate 3358 may be to a preset angle or may return the support 3352 all the way to a home position (see, e.g. FIG. 9A) in which the support 3352 is in line with the back plate 3358. Sidewalls 3362 may be included on the back plate 3352 to prevent displacement of the bag 26 in undesired directions.

Referring now to FIG. 10A-B, another example mix assisting assembly 2500 which displaces a bag 26 is depicted. The example mix assisting assembly of FIGS. 10A-B is arranged to rotate the bag 26 about an axis which extends parallel to the width dimension of the bag 26 and proximate the ports 392. As shown, the bag 26 may be held by a bag retainer 3350 which may be coupled to a carriage 3366 on a gantry 3368. The carriage 3366 may be displaced along the gantry 3366 from a first end of the gantry 3368 to the second end of the gantry 3368. The mix assisting assembly 2500 may also include a rest 3370. The rest 3370 may be a plate, bar, rod, or the like. The rest 3370 may be coupled to a linear displacement stage 3372. The rest 3370 may be brought to a stowed position at a first end of the displacement range of the linear stage 3372. The carriage 3366 may be displaced along the gantry 3368 to position a bag 26 in a ready position. As shown in FIG. 10A, the rest 3370 may then be brought into contact with the bag 26. The rest 3370 and carriage 3366 may then be displaced in coordination with one another to rotate the bag 26 about the rotation axis. As indicated in FIG. 10B, the rest 3370 may be displaced to a second end of its displacement range as the carriage 3366 is displaced toward the opposing end of the gantry 3368. Thus, the end of the bag 26 opposite the ports 392 may be raised while the end of the bag 26 including the ports 392 remains at substantially the same height. Preferably, rest 3370 may raise the end of the bag 26 opposite the ports 392 to a location higher than the ports 392. The carriage 3366 and rest 3370 may be displaced in reverse directions (back to their positions in FIG. 10A) to lower the bag 26. The control system 15 may issue commands to the linear displacement stage 3372 and gantry 3368 to repeatedly displace the bag 26 between raised and lowered states to engender mixing of fluid within the bag 26.

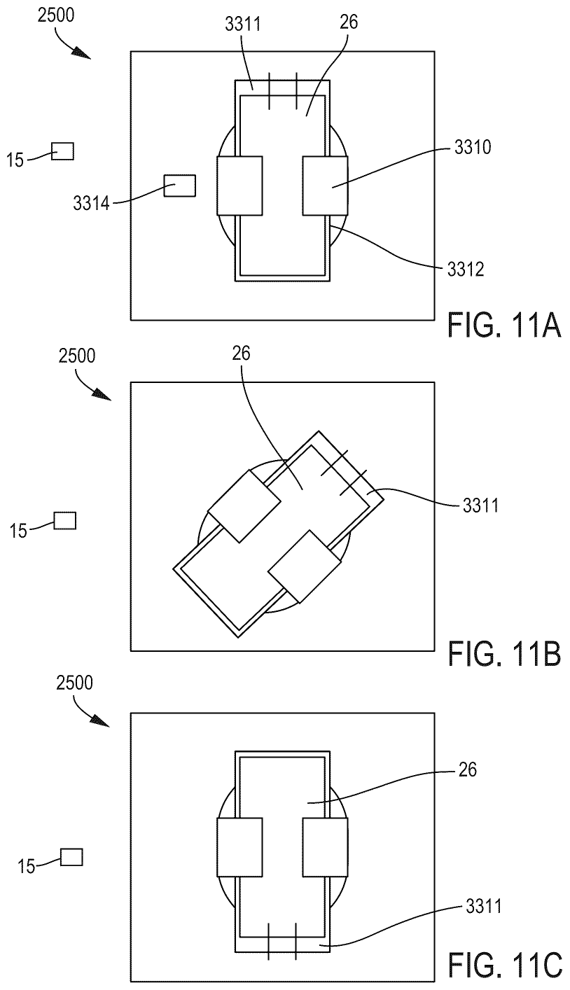

Referring now to FIGS. 11A-C, another exemplary mix assisting assembly 2500 is depicted. The example mix assisting assembly 2500 displaces the entire bag 26 to engender mixing of solution within the bag 26. As shown in FIGS. 11A-C, the bag 26 may be held in place on a rotary displacement stage 3312 included in the mix assisting assembly 2500. A bag retainer assembly 3310 may, for example, be coupled to the rotary displacement stage 3312 to capture the bag 26. Example bag retainer assemblies 3310 may have arms or clips which hold the bag 26 in place. A gripper or the like may be included to hold the ports 392 of the bag 26 in certain examples. A cradle 3311 or the like may also be included in the bag retention assembly 3310. The cradle 3311 may be coupled to the rotary displacement stage 3312 and may have a depression which is sized and shaped to accept at least a portion of a filled bag 26. The cradle 3311 and bag retention assembly 3310 may substantially prevent the bag 26 from displacing relative to the rotary displacement stage 3312. The gripper, clips, arms, etc. of the bag retention assembly 3310 may be mounted to the cradle 3311. One or more sensor 3314 which outputs a signal which varies in relation to the position of the rotary displacement stage 3312 may be monitored by a control system 15. The control system 15 may command displacement of the rotary displacement stage 3312 based upon the sensor 3314 output. In the example embodiment, the rotary displacement stage 3312 displaces the bag 26 such that the bag 26 rotates about an axis substantially perpendicular to a plane of the bag 26 in which the axes of the ports 392 fall. The rotation axis may be positioned so as to extend through a central location of the bag 26. The rotary displacement stage 3312 may be commanded to displace the bag 26 to one or more preset position. Typically, the bag 26 may be rotated to a sequence of predefined positions. The rotation may be alternated between clockwise and counterclockwise rotational directions between positions of the sequence of predefined positions. The bag 26 may be rotated at at least a certain rate as the bag 26 is rotated from one position to another. The bag 26 may remain stationary at each of the predefined positions for a period of time. In certain examples, the bag 26 may be rotated to a position at least 180° from its starting position (a fully inverted position) as the bag 26 is displaced with the mix assisting assembly 2500.

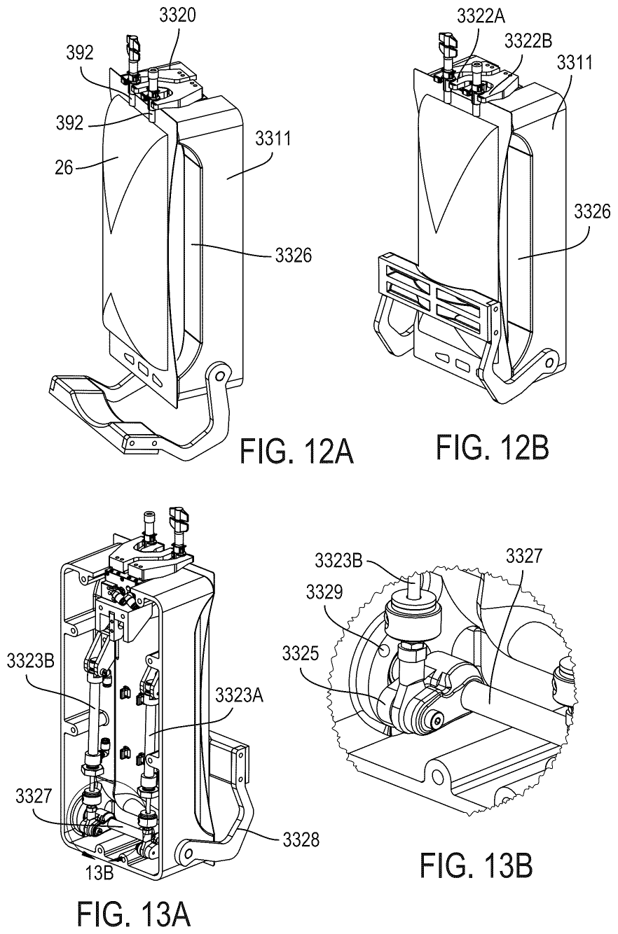

Referring now to FIGS. 12A-13B a number of views of an example bag retention assembly 3310 which may be included in a mix assisting assembly 2500 are depicted. As shown, the bag retention assembly 3310 includes a cradle 3311 which may include mounting points or hardware to couple the bag retention assembly 3310 to a rotary displacement stage 3312 (see, e.g., FIGS. 11A-B). The cradle 3311 may include a trough flanked by a set of opposing sidewalls 3326 which may support a bag 26 as the bag 26 is rotationally displaced. A gripper 3320 is attached to the cradle 3311 and may be actuated (e.g. pneumatically) to open and close a set of jaws 3322A, B for each port 392 included on the bag 26.

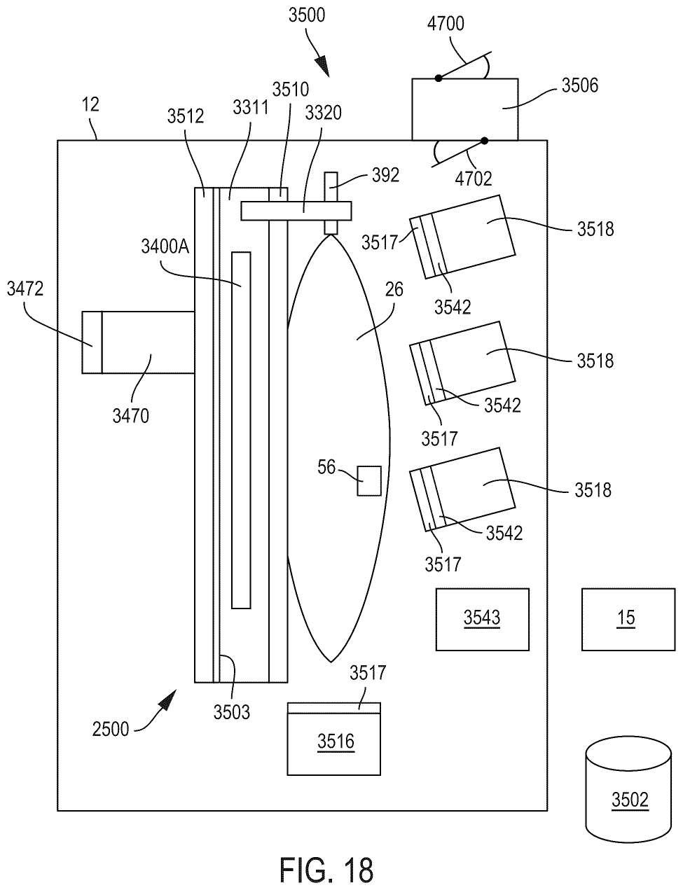

The bag retention assembly 3310 may also include a displaceable holder 3328 which may be displaced from a retracted position (see, e.g., FIG. 12A) and a deployed position (see, e.g., FIG. 12B). The holder 3328 may swing about an axis which runs substantially parallel to the width dimension of the bag 26. With the holder 3328 in the retracted position, the cradle 3311 may be accessible for a gantry or other robotic manipulate to install or remove a bag 26 from the mix assisting assembly 2500. Any imagers 3518 (see, e.g. FIG. 18) of a particulate inspection system 3500 (see, e.g. FIG. 18) may also have a clear field of view of the bag 26 when the holder 3328 is in the retracted position. In the deployed position, the holder 3328 may contact and press against the bag 26. This may firmly retain the bag 26 against the cradle 3311 to inhibit movement of the bag 26 relative to the cradle 3311 as the bag 26 is rotated. In some examples, the surface of the holder 3328 which contacts the bag 26 may be formed of or covered with a material with a high coefficient of friction against the bag 26 material. This may further assist in preventing undesired movement of the bag 26 as the bag 26 is rotated.

Referring now primarily to FIGS. 13A-B, the cradle 3311 may form a housing for one or more actuator 3323A, B (e.g. pneumatic cylinders) of the bag retention assembly 3310. A rear panel of the cradle 3311 is hidden in FIGS. 13-B to depict the interior of the housing. The rotary displacement stage 3312 (see, e.g., FIGS. 11A-C) may couple to the rear panel when assembled. The actuators 3323A, B may be driven to displace respective linkages 3325 coupled to a pivot body 3327 to which the holder 3328 is fixedly coupled. As the actuators 3323A, B act on the linkages 3325 rotation of the pivot body 3327 and thus displacement of the holder 3328 is engendered. One or more position sensor 3329 may be included to monitor the position of the holder 3328. A rotary encoder may for example output a signal which varies in relation to the rotational position of the pivot body 3327 in certain examples. Alternatively, where the actuators 3323A, B are pneumatic cylinders, a cylinder proximity sensor (reed switch, Hall effect sensor, magnetoresistive sensor (AMR or GMR), or other magnetic sensor monitoring a magnet coupled to the cylinder) may be used.

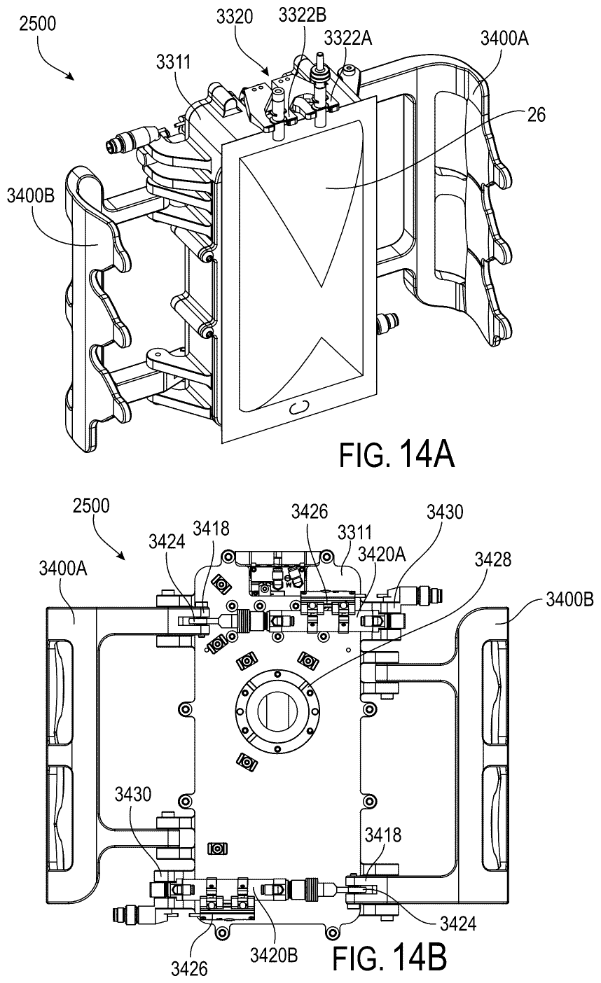

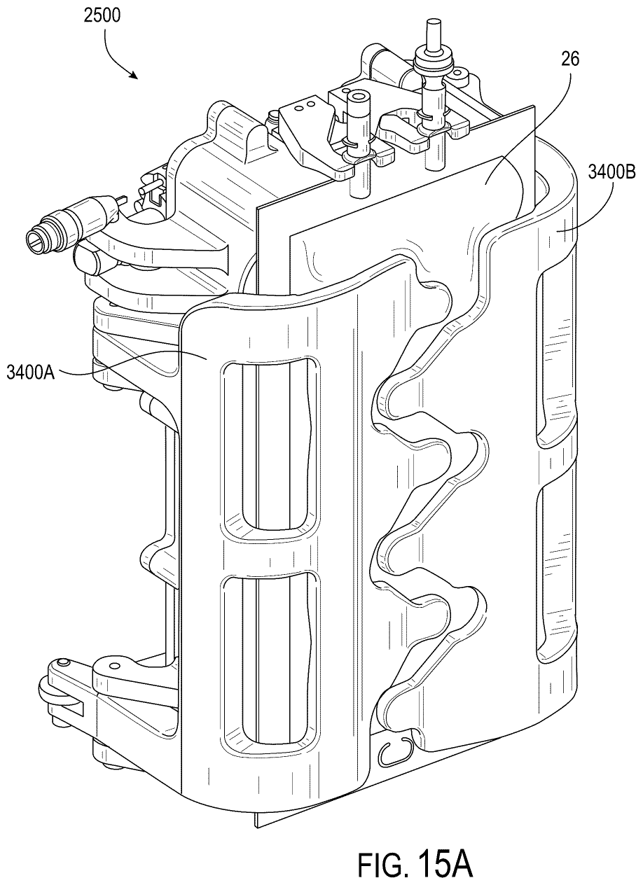

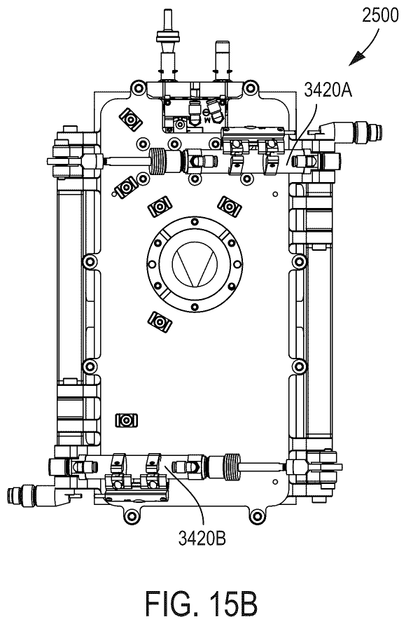

Referring now to FIGS. 14A-15B, another example of a bag retention assembly 3310 which may be included in a mix assisting assembly 2500 is depicted. As shown, the bag retention assembly 3310 includes a cradle 3311 in which bags 26 may be partially seated when in place at a mix assisting assembly 2500. A gripper 3320 may be coupled to the cradle 3311. The gripper 3320 may have jaws 3322A, B which may be actuated (e.g. pneumatically) to open and close around ports 392 of a bag 26. Additionally, the bag retention assembly 3310 includes a holder in the form of a set of doors 3400A, B. Each door 3400A, B may swing about a pivot point which runs substantially parallel to the longitudinal axis of the bag 26. The doors 3400A, B may be displaceable from an open state (see, e.g., FIG. 14A) to a closed state (see, e.g., FIG. 15A). In the open state, a robotic manipulator or gantry may have a clear path to deposit or retrieve a bag 26 from the bag retention assembly 3310. In the closed state the bag 26 may be firmly held against the cradle 3311. In some examples, the doors 3400A, B may also be displaceable to an third, overly closed position. With a bag 26 present in the bag retention assembly 3310, the bag 26 may present an interference to displacement of the doors 3400A, B to this third position. The control system 15 may monitor the position of the doors 3400A, B to confirm a bag 26 is appropriately in place in the bag retention assembly 3310. One or more sensor may be included for this purpose (e.g. at least one sensor may monitor each door 340A, B). In the event that a bag 26 is expected to be present and a door position sensor indicates one or more door 3400A, B has reached the third position (or is within a predefined range of the third position), the control system 15 may generate an error notification. This may indicate that a bag 26 is improperly retained in the bag retention assembly 3310 or a bag 26 is absent when expected to be present.

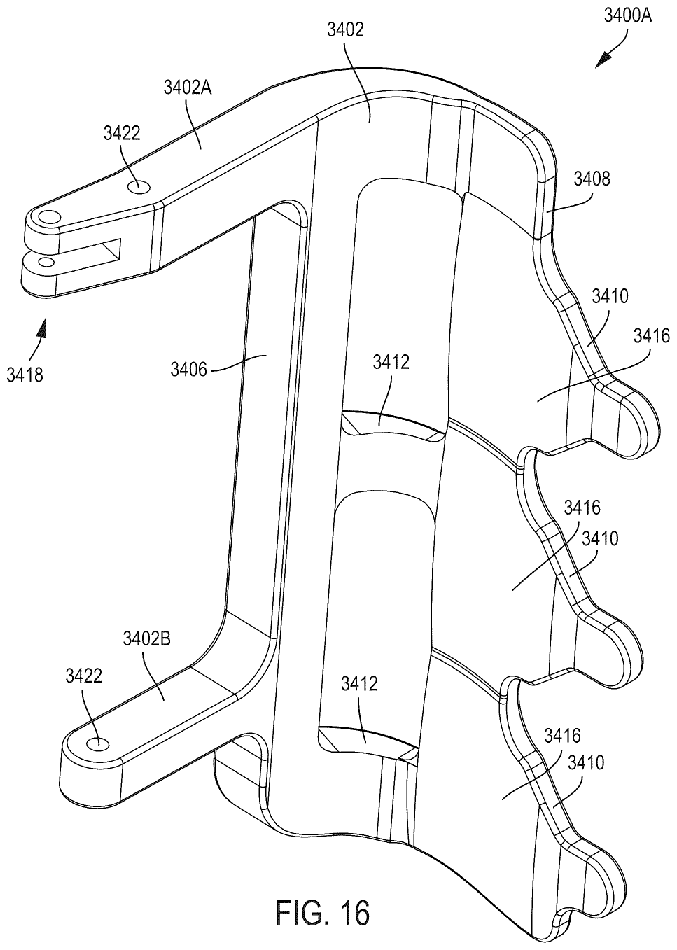

Referring now also to FIG. 16, a perspective view of a door 3400A in isolation is depicted. Each of the doors 3400A, B may be identical to one another for sake of simplicity, though this need not be the case in all examples. In examples where the doors 3400A, B are identical, the doors 3400A, B may be installed in a state where they have been flipped 180° from one another on opposing sides of the cradle 3311. The doors 3400A, B may be coupled to the cradle 3311 so as to swing in symmetric displacement paths lateral to the cradle 3311.

Still referring to FIGS. 14A-16, each door 3400A, B may include a main body 3402. The main body 3402 may, though need not, include one or more aperture 3412. A set of arms 3404A, B may project from opposing end regions of a first side 3406 of the main body 3402 of each door 3400A, B. One arm 3404A may be longer than the other and include an actuator coupling 3418 at its terminal end for coupling the arm 3404A to the output 3422 of a door actuator 3420A, B. Each arm 3404A, B may include a pivot pin hole 3422 via which the respective arm 3404A, B may be pivotally coupled to the cradle 3311. The pivot pin holes 3422 may be substantially coaxial.

A plurality of protrusions 3410 may extend from a second, opposing side 3408 of the main body 3402. The protrusions 3410 may be spaced apart by gaps which mimic the shape of the protrusions 3410. Thus, when the doors 3400A, B are closed (see, e.g., FIG. 15A), the protrusions 3410 of each door 3400A, B may be received in the gaps between the protrusions 3410 of the opposing door 3400A, B. This interlocking arrangement may assist in preventing pinching of the bag 26 when the doors 3400A, B are actuated. In the example embodiment, each door 3400A, B includes three protrusions 3410. All corners on the protrusions 3410 may be rounded. Each protrusion 3410 may include a base region. A rounded transition may be present between the base region and the end of the door 3400A, B from which the protrusion 3410 projects. The base region of the protrusions 3410 may taper thinner and include a rounded transition to a nub at their terminal ends. The tip of the nub of the protrusions 3410 may also be rounded. The doors 340A, B may generally taper thinner in thickness as proximity to the side of the doors 3400A, B including the projections 3410 increases.

As shown, the main body 3402 may be arcuate in cross section such that the arms 3404A, B extend in a direction which is 80-100° from the direction of extension of the protrusions 3410. This arcuate shape may assist in providing a rolling contact against the exterior surface of the bag 26 as the doors 3400A, B are displaced to a closed position against the bag 26. A portion of the main body 3402 and protrusions 3410 may include a contact surface which abuts a bag 26 in the mix assisting assembly 2500 when the doors 3400A, B are in a closed state. As shown, one or more panel of gripping material 3416 which has a high friction coefficient with respect to the bag 26 may be placed on the contact surface. Additionally, the protrusions 3410 may at least partially extend in a plane which is offset from the second side of the main body 3402. When the doors 3400A, B are in a closed state, the portion of the doors 3400A, B formed by the protrusions 3410 may thus be more proximate the midplane of the bag 26 in which the ports 392 fall. Force exerted by the doors 3400A, B against the bag 26 may thus be concentrated along a center line of the bag 26. This may depress the medial portion of the bag 26 pushing fluid within the bag 26 laterally.

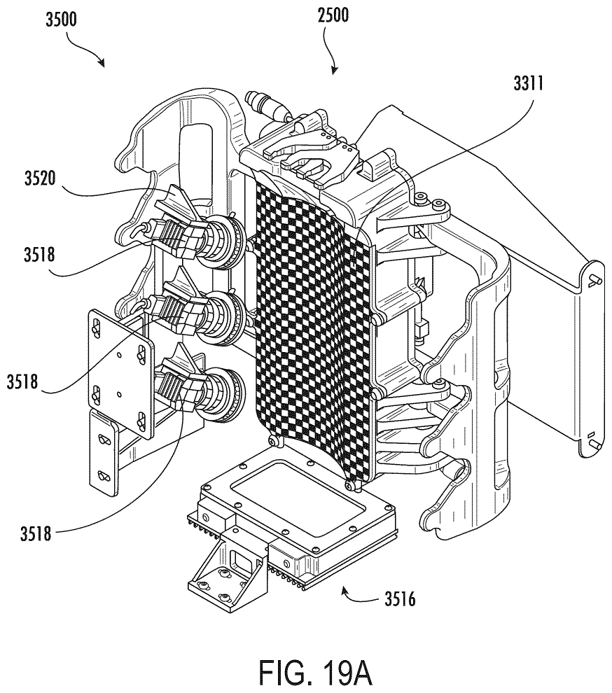

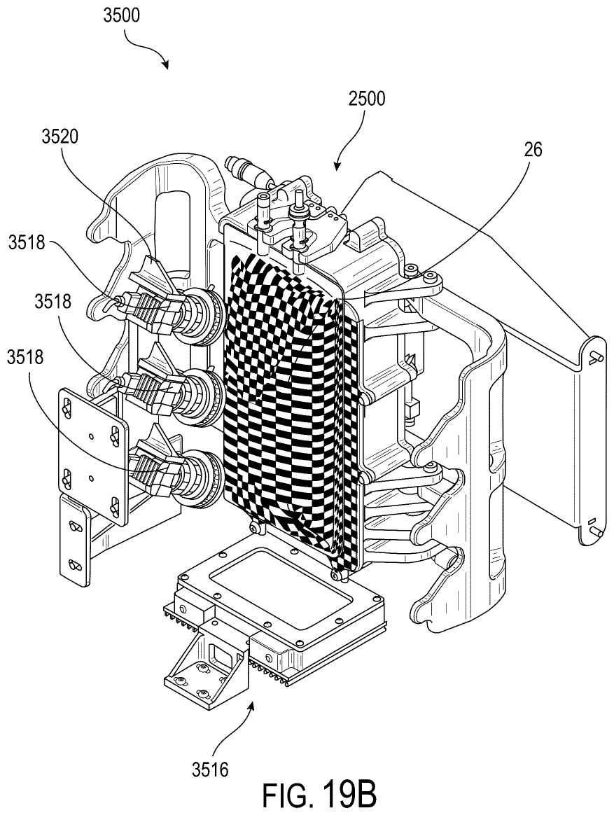

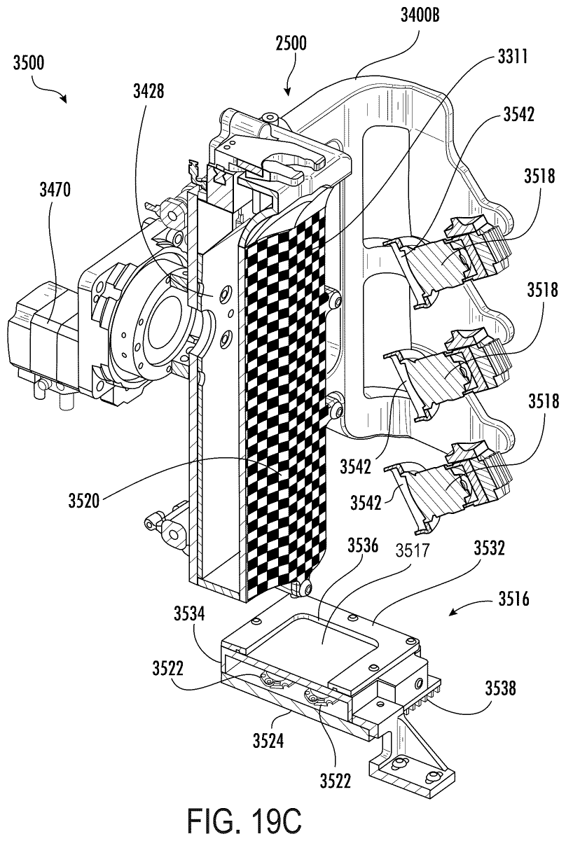

As shown best in FIG. 14B and FIG. 15B, the output 3424 of each door actuator 3420A, B may be coupled to an actuator coupling 3418 on a respective door 3400A, B. In the example, the outputs 3424 are pivotally coupled such that the respective door 3400A, B may pivot relative to the output 3424 to which it is coupled. Each door actuator 3420A, B may also be pivotally coupled to the cradle 3311. Each door actuator 3420A, B may pivot relative to the cradle 3311 as the door 3400A, B is driven through its displacement range by the door actuator 3420A, B. In the example embodiment, the end of each door actuator 3420A, B opposite the output 3424 is coupled to a hinge 3430 extending laterally from the cradle 3311. A position sensor 3426 may be associated with each door actuator 3420A, B and may output a signal indicative of the position of the respective door 3400A, B as the door actuator 3420A, B is driven. This data signals from these sensors 3426 may be monitored by the control system 15 to determine whether the doors 3400A, B are in the open, closed, or in an overly closed position as mentioned above. In the example, the door actuators 3420A, B are pneumatic actuators and the position sensors 3426 may be magnetic sensors tracking the position of a magnetic body on the pneumatic cylinders. Any such sensor variety described herein may be used. Additionally shown in FIG. 14B and FIG. 15B is a mount point 3428 for coupling of the cradle 3311 to a rotary displacement stage 3312 or assembly. The mount point 3428 is shown coupled to a rotary actuator 3470 in FIG. 19C for example.

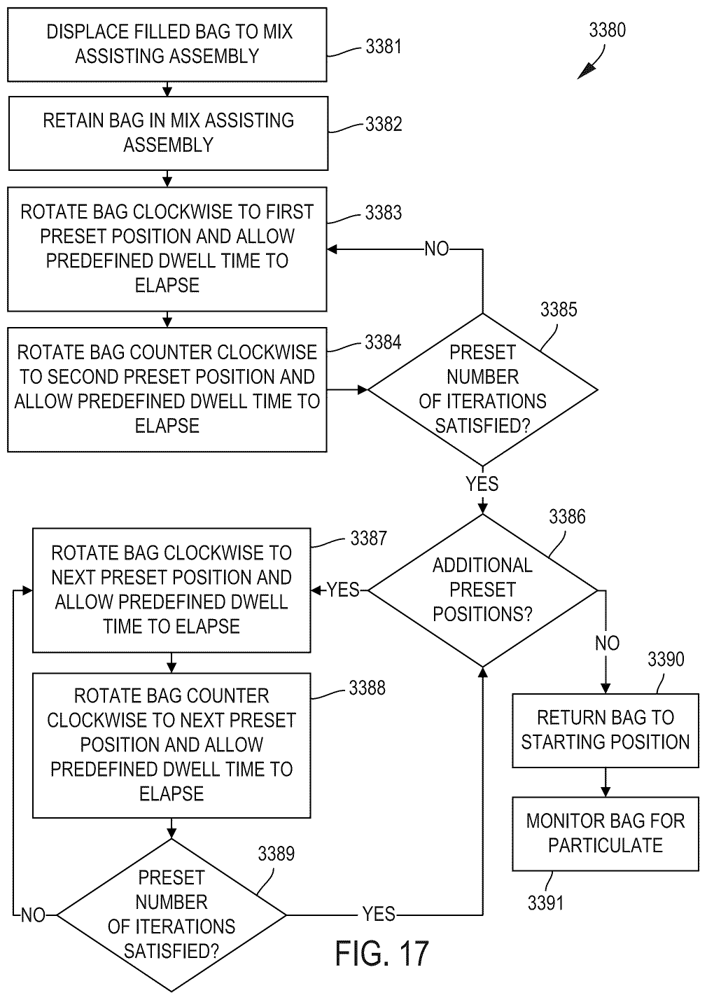

Referring now to FIG. 17, a flowchart 3380 is depicted detailing a number of example actions which may be executed to displace a bag 26 to encourage creation of a homogenous solution within the bag 26. As shown, in block 3381 a bag 26 may be displaced to the mix assisting assembly 2500. This may be accomplished by a robotic gripper or gantry under direction of the control system 15. In block 3382, the bag 26 may be retained in the mix assisting assembly 2500 by a bag retention assembly 3310.

In the example shown in FIG. 17, the bag 26 may be rotated between pairs of preset clockwise and counterclockwise positions. The order in which the bag 26 is displaced to the clockwise position and counterclockwise position in each pair may differ from embodiment to embodiment. The bag 26 may be displaced in a first rotational direction to the first position of a pair and rotated in a second, opposing rotational direction to the second position in that pair. FIG. 17 describes displacing the bag 26 to the clockwise position and then to the counterclockwise position though this merely exemplary.

The bag 26 may be rotated between each position in a pair some preset number of times. For some pairs, the number of iterations may be only a single iteration. For other pairs, the bag 26 may be rotated between each position in a pair a plurality of times (e.g. three times). A control system 15 may govern operation of a rotary actuator 3470 (see, e.g. FIG. 18) based on data from at least one rotational position sensor 3472 (see, e.g., FIG. 18) to rotate the bag 26 to any preset positions. The rotation rate of the bag 26 may be controlled to a predefined rate. In certain examples, the bag 26 may be rotated at a rate of 200-300 degrees per second (e.g. 250 degrees per second) during transitions between all positions. In certain examples, the bag 26 may be displaced between preset positions at rates specific to each individual transition between positions. The specified rates may differ depending on the transition, though at least some of the individual transitions may be specified to occur at the same rate. There may be a dwell period where the bag 26 is kept substantially stationary. This dwell period may elapse after the bag 26 is rotated to each preset position. In the example, a predefined dwell period is allowed to elapse each time the bag 26 is displaced to a new position (see, e.g., blocks 3383, 3384, 3387, 3388). The dwell time period may be specified for each individual displacement of the bag 26 or a dwell time for use with each pair of positions may be specified in non-limiting examples. A set of position pairs, rotation rates, and dwell times may be referred to as a motion profile.

In some examples, the type of bag 26 in place at the mix assisting assembly 2500 may be known. For example, the bag 26 may have an identifier (see, e.g., indicium 56 of FIG. 18) which is read by an imager, barcode reader, etc. in communication with the control system 15. Bag 26 identifying information may also be manually input via a user interface in certain examples. The motion profile used for a given bag 26 may be chosen by the control system 15 based on the type of bag 26 in place at the mix assisting assembly 2500. A look-up table or the like may be used to facilitate selection of a motion profile based on a bag 26 identity. For example, the position pairs may be held constant across bags 26. Maintaining the same dwell times for each rotation pair, the rotational rate may be increased as the volume of the bag 26 increases. Alternatively, dwell times could be increased while maintaining the rotational rate constant for each position pair as bag 26 volumes increase. It is not necessary that only one of the dwell time and rotation rate values be adjusted; certain implementations may adjust both values as bag 26 volume increases.

Without being tied to any particular theory, it is believed that rotating the bag 26 to a given position generates primary or initial eddies within the fluid contained in the bag 26. The size of the primary eddies may generally be a factor of the amount and rate of angular displacement for a given reservoir (e.g. bag 26). As the bag 26 dwells in position, the primary eddies may decay into smaller eddies due to inertial instabilities. These eddies have a smaller Reynolds number and thus are less responsive to changes in momentum of the bag 26. These eddies may primarily respond to the viscosity of the surrounding solution rather than rotation of the bag 26. When the bag 26 is rotated to the opposing position of a position pair, the intervening dwell period may allow more of the energy from the counter-rotation to go towards creating new primary eddies instead of devoting some of the energy to slowing down existing primary eddies. Additionally, the remaining smaller eddies from previous preset positions may feed into the advective mixing action engendered by the primary eddies induced as the bag 26 is rotated to subsequent preset positions. As the bag 26 continues to be rotated between positions, the eddies formed within the bag 26 may mix fluid within the bag 26 with greater and greater vigor. The primary eddies formed may act on the fluid in conjunction with the mixing effects provided by secondary, tertiary, quaternary, etc. eddies from previous rotations. Additionally, as the bag 26 is rotated a greater magnitude from the starting position, the primary eddies formed may become larger resulting in larger child eddies (and longer cascades of progeny child eddies) as they break down. Thus repeated displacement of the bag 26 between positions of increasing (though not necessarily strictly increasing) magnitude may efficiently and quickly homogenize fluid within a bag 26 and dissolve any solid constituent within the bag 26.

Still referring to FIG. 17, the bag 26 may be rotated clockwise from a starting position to a first preset position in block 3383. The bag 26 may be rotated counterclockwise to a second preset position in block 3384. In some examples, the first preset position may be 40° from the starting position and the second preset position may be −40° from the starting position. The magnitude of rotational displacement from the starting position for the clockwise and counterclockwise position for each position pair may not be equal in all embodiments. In certain examples, the bag 26 may dwell at each of the first and second preset positions for less than half a second (e.g. 0.275-0.325 seconds or 0.390-0.410 seconds) for a one liter bag 26. The control system 15 may determine any dwell times (and/or rotation rates) based on bag 26 volume in certain embodiments (e.g. via a look up table).

If, in block 3385, the preset number of displacement iterations between the first and second position has not been met, the bag 26 may again be rotated between the first and second position in blocks 3383, 3384 and the accompanying dwell periods may be allowed to elapse. In some examples, the bag 26 may be displaced three times between the first and second positions. In certain implementations, the number of iterations may depend on the type of concentrate included in the bag 26. A greater number of iterations may be used for bags 26 which were filled with solid constituent. Additionally, a greater number of iterations may be used in the event that the bag 26 was filled with liquid concentrate and a particulate inspection system 3500 (see, e.g., FIG. 18) determined the presence of solid precipitate.

After the preset number of displacement iterations has been satisfied in block 3385, the bag 26 may be rotated clockwise and counterclockwise to the next preset positions in blocks 3387, 3388 (if in block 3386 there are additional preset positions). Dwell periods associated with the positions for blocks 3387, 3388 may be allowed to elapse. The bag 26 may be rotated between this next pair of positions until a preset number of displacement iterations has been achieved in block 3389. This may repeat until the bag 26 has been displaced to all predefined positions.

Typically, the magnitude of rotational displacement may increase with each subsequent pair of preset positions. The dwell time may also increase as the magnitude of displacement increases. In certain embodiments, the bag 26 may be rotated to a third and fourth position. The third position may be 85° from the starting position and the fourth position may be −85° from the starting position. The bag 26 may dwell at each of the third and fourth position for less than 0.75 seconds (e.g. 0.6-0.7 seconds). The bag 26 may also be rotated to fifth and sixth positions. The fifth position may be 120° from the starting position and the sixth position may be −120° from the starting position. The bag 26 may dwell at each of the fifth and sixth position for less than a second (e.g. 0.775-0.825 seconds). The bag 26 may further be rotated between seventh and eight positions. The seventh position may be 180° from the starting position and the eighth position may be −180° from the starting position. The dwell time at each of the seventh and eighth positions may be less than seven seconds (e.g. 4-6 seconds). In other examples, a lesser or greater number of positions pairs may be used.

The number of displacement iterations between each pair of positions may decrease (though non-strictly in certain examples) as the magnitude of rotational displacement increases. The number of displacement iterations between the last pair of positions may be lower than the number of displacement iterations for the first pair of positions. At least for bags 26 where the constituent within the bag 26 is a crystalline solid or powder, the number of displacement iterations may be greater for pairs of positions where the bag 26 is rotated less than 90° from its starting position. This may prevent constituent from displacing and lodging into a port 392 of the bag 26.

Still referring to FIG. 17, once, in block 3386, the bag 26 has been displaced through all preset positions, the bag 26 may be returned to the starting position in block 3390. Depending on the embodiment, the bag 26 may be monitored for particulate in block 3391. A wait period may optionally be allowed to elapse prior to any such monitoring. A control system 15 may, for example, analyze one or more image of the bag 26 supplied by one or more imager 3518 (see, e.g., FIG. 18) to determine whether particulate is present. Particulate monitoring will be further described later in the specification (see, e.g., FIG. 18). The bag 26 may also be checked to determine whether any solid constituent present in the bag 26 has dissolved in block 3391. In certain embodiments, the bag 26 may be rotationally displaced to further positions in the event that undissolved constituent is determined to be present within the bag 26. This may repeat until the control system 15 determines all solid constituent in the bag 26 has been dissolved in block 3391.

In alternative embodiments, the bag 26 may be rotated between a set of positions or sets of positions prior to block 3383 and the bag 26 may be subsequently monitored for particulate. This may create movement of particulate within the bag 26 which may assist in detection of the particulate with a particulate detection system 3500 (see, e.g., FIG. 18). The number of rotation iterations for each position pair may be adjusted by the control system 15 based upon the particulate analysis. The control system 15 may also halt operation of the mix assisting assembly 2500 in the event that certain undesired particulate is determined to be present. For example, in the event that insoluble foreign particulate is detected, the control system 15 may flag the bag 26 as rejected and the bag 26 may be directed to quarantine without mixing.

Referring now to FIG. 18, a diagram of an example particulate inspection system 3500 is depicted. Example particulate inspection systems 3500 may be implemented as stand-alone systems or be integrated into other systems. Additionally, the example particulate inspection systems 3500 are described in relation to flexible reservoirs (e.g. IV bags) typical of the variety used in in medical settings. These reservoirs are generally described in the context of injectable fluids. This is merely exemplary. The example particulate inspection systems 3500 described herein may be utilized with other varieties of reservoirs used in a wide variety of applications. For example, rigid reservoirs such as vials, jars, bottles, etc. may be used. The example particulate inspection systems 3500 described herein may also be used with reservoirs which include both rigid and flexible portions. Reservoir retention hardware of the particulate inspection systems 3500 may be adapted to retain the desired reservoir variety. Additionally, the displacement profile during mixing may be adjusted based on the subject reservoir. Applications may include preparation of medical solutions, laboratory solutions, and biological applications such as cell culturing, biomanufacturing, etc. Applications may also include particulate detection for industrial products (e.g. oils conforming to a stringent ISO cleanliness code). Examples described herein are generally described in relation to liquids, though may be used to detect particulate in other fluids such as gases. Though referred to herein as particulate inspection systems 3500, it will be clear that such systems are also capable of detecting, for instance, bubbles and may be utilized as bubble detection systems for reservoirs. Particulate inspection systems 3500 may also be referred to herein as reservoir content detecting and/or differentiating systems. Particulate inspection systems 3500 described herein typically detect, track, and classify contents of interest within reservoirs. In certain alternatives, a particulate inspection system 3500 may perform a subset of these activities. Some particulate inspection systems 3500 may detect and optionally classify components of interest without necessarily tracking them.

In general, particulate inspection (or content detection and differentiating in general) of reservoirs is a particularly difficult challenge. Commonly it is accomplished with a human inspector which engenders a high degree of subjectivity. Best practices require frequent breaks, recurring training, and other recurring reverification (e.g. eye exams) for inspectors. Even so, FDA data indicates that around one third of recalls for sterile injectables are related to the presence of particulate. Within the time period of 2010-2021 there were between 6 and 25 recall events per year relating to visible particulate in sterile injectables. A relatively small footprint automated particulate inspection system which is not prohibitively expensive would be welcome.

Particles or other content of interest for identification may be quite small. For example, it may be desired that a particulate inspection system 3500 identify particles of 200 μm or less (e.g. 100 μm or less or even 50 μm or less) at a level which at least achieves parity with a human inspector. These particles may exist in a solution which also contains gas bubbles. Some microbubbles within the solution may be in the same size range as the particulate of interest making differentiation between particulate and bubbles a challenge. False rejection of a reservoir due to misidentification of an air bubble as particulate is undesirable. It may be particularly undesired in scenarios where storage for rejected reservoirs is limited and is access controlled or in an environmentally controlled enclosure. Excessive false rejection may require a user intervention with a fluid packaging system and may limit throughput of a parent system which produces and packages fluids.

Adding to the challenge, certain particulate may possess properties which are not ideal for visual detection. Some particulate (e.g. particulate from IV bag material) may be substantially transparent or have a high degree of translucence. Other particulate may absorb or reflect substantial amounts of light making it difficult to view against certain backgrounds. The density of particulates may also present challenge as certain particulate may tend to collect at the top or bottom of a reservoir. Head space in a reservoir may further complicate detection. The type of reservoir can also present optical challenges. IV bags, for example, are flexible and malleable. Each bag 26 will tend to crease and seat differently when installed in a particulate inspection system 3500 giving respective bags 26 differing optical properties. Reservoirs may also include print (e.g. text, logos, etc.) which may obfuscate view into the reservoir. Some reservoirs such as IV bags may also display a tendency to settle or shift over time while observations are made.

Exemplary particulate inspection systems 3500 described herein may image a filled bag 26 and perform machine vision analyses to identify contents of interest within the fluid contained in a bag 26. Particulate inspection systems 3500 may also differentiate between different types of contents of interest within the fluid. Bubbles of air or gas may be differentiated from solid particulate for example. In certain embodiments, particulate inspection systems 3500 may also differentiate between different types of particulate. A particle of undissolved constituent (e.g. salt, sugar, etc.) may be differentiated from other types of particulate for instance. Any determination as to the presence of at least certain contents of interest may be documented in a log which may be committed to a database 3502 or other memory. A determination as to whether the subject bag 26 meets one or more acceptance criteria may also be made. This determination, any raw data, analyzes, digests thereof, summaries, etc. may be included in the log communicated to the database 3502. In the event that the bag 26 fails acceptance criteria, the bag 26 may be routed to a discard or quarantine destination in a parent system for producing and packaging fluids. This could for example be dedicated, access controlled receptacles 4120B or an outfeed drawer in a parent system. The bag 26 may fail acceptance criteria in the event that analysis is indicative that undesired contents of interest such as particulate are determined to be present in the bag 26.

As shown, example particulate inspection systems 3500 may be disposed within an enclosure 12. The enclosure 12 may be substantially light tight. Thus ambient light may be obstructed from reaching the interior of the particulate inspection system 3500. The interior of the enclosure 12 may be unlit, but for light generated by components of the particulate inspection system 3500. In some embodiments, the enclosure 12 may house one or more additional station of a system 10 (e.g. a marking/labeling assembly, outfeed assembly, etc.). In such embodiments, light generating components in these stations may be minimized. There may for example be one or more small, low lumen output LED indicator light on components in the enclosure 12 while still considering the enclosure 12 to be unlit. To the extent other stations which utilize lighting are present within the enclosure 12, the particulate inspection system 3500 may not image bags 26 coincident with lighting in other stations being powered. Shielding or masking of light emitting components within the enclosure 12 may also be implemented. The particulate inspection system 3500 may be within a partitioned portion of the enclosure 12 separate from light producing components. An irradiation assembly with antimicrobial light emitters may be in a processing compartment of a system while the particulate inspection system 3500 is in a separate compartment for example.

The enclosure 12 may be environmentally controlled to limit microbes, detritus, and humidity in certain examples. Where the particulate inspection system 3500 receives bags 26 from a fill station, the enclosure 12 in which the particulate inspection system 3500 is disposed may be environmentally controlled to a less stringent level than the enclosure 12 in which the fill station is disposed. Alternatively, the enclosure 12 may not be subjected to environmental control. A transfer chamber 3506 may be disposed intermediate a processing compartment of the enclosure 12 including the fill station and compartment of the enclosure 12 including the particulate inspection system 3500. The transfer chamber 3506 may include a set of doors 4700, 4702 which may opened via respective actuators under control of the control system 15. The control system 15 may inhibit both of the doors 4700, 4702 from being in an open state at the same time.

The particulate inspection systems 3500 may include a retainer assembly for holding a bag 26. In the example embodiments described herein, the retainer assembly is described as a mix assisting assembly 2500 (further described in relation to FIGS. 14A-17). Where a mix assisting assembly 2500 is described as part of a particulate inspection system 3500, it shall be understood that this is merely exemplary. The retainer assembly of a particulate inspection system 3500 may be a dedicated assembly and certain embodiments may include a separate mix assisting assembly 2500. Features of mix assisting assemblies 2500 described in relation to example particulate inspection systems 3500 disclosed herein may be included in a dedicated retainer assembly for a particulate inspection system 3500. Additionally, any of the features of the mix assisting assemblies 2500 described in relation to the particulate inspection system 3500 may be included in any other mix assisting assemblies 2500 shown or described herein.

As shown, the mix assisting assembly 2500 may include a cradle 3311 against which the bag 26 may be placed. A gripper 3320 may be attached to the cradle 3311 and may be actuated (e.g. pneumatically) to open and close a set of jaws around respective ports 392 of the bag 26. The cradle 3311 may be attached to a rotary actuator 3470 via a mount point 3428 (see, e.g., FIG. 14B). A motor encoder or other rotational position sensor 3470 may be included and may output a signal indicative of the rotational position of the cradle 3311. A holder may be attached to the cradle 3311. Any holder described herein may be used, though in the example embodiment, a set of doors 3400A, B (only one shown in FIG. 18, see, e.g., FIG. 14A) are included. The holder may be actuated to firmly retain the bag 26 against the cradle 3311 when the rotary actuator 3470 is driven.

The cradle 3311 may include a rest body 3510 which a surface of the bag 26 may contact when retained at the cradle 3311. The rest body 3510 may be contoured to mimic the shape of a filled bag 26. The rest body 3510 may be transparent or at least partially translucent. For example, rest bodies 3510 may be constructed of a transparent or at least partially translucent material. Alternatively, the rest body 3510 may be constructed of a transparent material to which a surface treatment is applied. The rest body 3510 may, for instance, be frosted. The cradle 3311 may also include an illuminator 3512. The illuminator 3512 may include at least one light source and may provide substantially uniform illumination of the rest body 3510. A diffuser may also be included. In certain examples, a polarizer 3503 may additionally or instead be included. When the illuminator 3512 is powered, a bag 26 in place on the cradle 3311 may be backlit. As shown in FIG. 18, the illuminator 3512 is positioned opposite the rest body 3510. Alternatively or additionally, the rest body 3510 may be side lit and/or otherwise lit from another vantage point. The illuminator 3512 may be a side emitting panel type light source with a built in diffuser. The illuminator 3512 may emit white light, though in some embodiments, the illuminator 3512 may emit specified wavelengths or may be adjusted to emit a selection of different color light(s) under the direction of a processor of the control system 15. The illumination output intensity of the illuminator 3512 may be adjustable.

The rest panel 3510 may be patterned. The pattern may include a collection of dark 3514A and light regions 3514B (see, e.g., FIG. 19A). The dark regions 3514A may be formed by masking or otherwise obstructing the passage of light through the rest body 3510. Thus, in various examples, the rest panel 3510 may include a collection of light obscuring regions and regions which permit passage of light. The dark regions 3514A may be black when the illuminator 3512 is powered. The light regions 3514B may be left uncovered or be unaltered regions of the rest body 3510. The light regions 3514B may be white (or substantially the color output by the illuminator 3512) when the illuminator 3512 is powered. The pattern may consist of alternating dark and light regions 3514A, B. In certain examples, a checker or chessboard pattern may be used. Other example patterns may include spirals, horizontal lines, vertical lines, polka dot, herringbone, houndstooth, a tessellation, zebra-pattern stripes, etc. In some preferred embodiments, a checkered pattern with 6-8 mm squares (e.g. 7 mm) may be utilized. The illuminator 3512 may light the rest panel 3510 at a value of at least 3000 lux.

In alternative embodiments, the illuminator 3512 may be otherwise controlled to generate contrast. For example, the illuminator 3512 may be powered to generate contrast in backlight color over time. In one example, no pattern may be included on the rest panel 3510, and the illuminator 3512 may be rapidly switched on and off, transitioned through different colors, or transitioned through different light output intensities at a high rate. This would similarly generate a contrasting light and dark background albeit with a temporal offset. The speed at which different illuminator 3512 set points are transitioned through may be dependent on the frame rate of any imagers 3518. The frame rate of the imagers 3518 may similarly be synchronized with the illumination transitions of the illuminator 3512. A transition between illuminator 3512 set points may occur a plurality of times per second. In some examples, ten or more transitions per second may occur.

In still other examples, a pattern (e.g. a chessboard pattern or any other pattern mentioned herein) may be produced on a display. In such examples, the pattern may be dynamic and may change during imaging by the imager 3518. In certain examples, the display may switch from a first pattern to a second pattern (and potentially other patterns). Though described as patterns, it would also be possible to use one or more solid background of a specific color (e.g. white, black, some preset color space value) in the series of transitions. The color(s) of a solid background (or portion(s) of a patterned background) may change at different time points. The same pattern may also be adjusted such that it is reduced/enlarged. For example, squares of a chessboard may be increased or decreased in size. A first pattern may be used for a first series or set of images and a second pattern may be used for a second series or set of images (and so on if desired). The first and second sets of images may be sets of consecutive images, but need not be in all examples. Any changes may be synchronized with the frame rate to ensure that the desired pattern is displayed for frames which will belong to a given set. Each pattern may be tuned for a particular content type (e.g. for certain varieties of particulate or for different content size thresholds).

An external illuminator 3516 may also be included in certain examples. The external illuminator 3516 may be separate from the cradle 3311 and may be positioned to direct light into the bag 26. In the example embodiment, the external illuminator 3516 is an underlight. The external illuminator 3516 may emit collimated light or have light sources which emit light in a narrow beam angle. In certain examples, the external illuminator 3516 may include a number of light emitters (e.g. LEDs) and one or more lens. The lens may include collimating or Fresnel lenses for each of the light emitters such that the external illuminator light 3516 sources emit collimated light. It should be understood that the term collimated would encompass light output from such an arrangement and should not be interpreted as meaning strictly perfectly collimated. Narrow beam angle lenses may also be used. Where narrow beam angle is used herein it refers to beam angles of 30° of less (e.g. no greater than 20°). In other examples the external illuminator 3516 may include light sources with wider beam angles. Additionally, in certain examples, at least one polarizer 3517 may be included. A polarizer 3517 may be included for the external illuminator 3516 and for each of the imagers 3518. Polarized light reduces glare off of certain reservoirs such as bags 26. Polarized light may also tend to better highlight air bubbles within a reservoir.

The external illuminator 3516 may produce light of a specific color, wavelength, or band of wavelengths. For example, the external illuminator 3516 may produce red or orange light. In some embodiments, the external illuminator 3516 may output light in the 600-630 nm range. The color of light selected for the external illuminator 3516 may be chosen based on potential anticipated sources of particulate. Preferably, the color may be selected to differ from anticipated sources of potential particulate. The intensity of the light emitted by the external illuminator 3516 may be greater than the intensity of light emitted by the illuminator 3512 in the cradle 3311. In alternative examples, the color of light emitted by the external illuminator 3516 may be adjustable and may be user specified. Different colors may be emitted by the external illuminator 3516 at different time points during inspection of a bag 26 or other reservoir. The external illuminator 3516 may produce an illuminance of at least 20,000 lux (e.g. 23,000-25,000 lux). Each light emitter assembly 3522 (see, e.g., FIG. 19C) of the external illuminator 3516 may also emit at a narrow beam angle such as 5-15° (e.g. 10°).

Still referring to FIG. 18, exemplary particulate inspection systems 3500 may include at least one imager 3518. Each of the at least one imager 3518 may be positioned in opposition to the rest body 3510 such that a bag 26 is disposed intermediate the rest body 3510 and imager(s) 3518 when in place at the cradle 3311. Thus the pattern may be provided on a first side of the reservoir (e.g. bag 26) while the imager(s) 3518 may be positioned on a second, opposing side of the reservoir. Each of the at least one imager 3518 may include a CCD or CMOS sensor and may capture monochrome or color image data. Each imager 3518 may have a depth of field which is at least equal to the thickness of a filled bag 26. The f-number for the imager(s) 3518 may be kept under f-8 and preferably lower. This may limit the amount of diffractive blur introduced to any images generated by the imager(s) 3518. Each imager 3518 may preferably have an at least 10 megapixel camera (e.g. 12 megapixel). Each pixel may correspond to a square region at the midplane of a nominal bag 26 which includes the ports 392. For exemplary 1 liter bags 26, a 100 μm particle may fill a pixel of an image generated by an example imager 3518.

In the example embodiment shown in FIG. 18, three imagers 3518 are included. The imagers 3518 are positioned at an angle other than perpendicular to the midplane of the bag 26 including the ports 392. In the example, the imagers 3518 are disposed such that the optical axis or viewing axis of each imager 3518 is inclined 30-35° (e.g. 32°)from an orientation perpendicular to the midplane of the bag 26 including the ports 392 or height dimension of the cradle 3311. A hypothetical spherical lens within the bag 26 receiving collimated light from an underlight type external illuminator 3516 may produce a light return which is particularly strong at this angle.

The imagers 3518 may each have a field of view which includes a portion of the bag 26. A first of the imagers 3518 may have a field of view including a portion of the bag 26 most proximal to the ports 392. Another of the imagers 3518 may have a field of view encompassing a portion of the bag 26 most distal to the ports 392. The remaining imager 3518 may have a field of view which captures the central portion of the bag 26 intermediate the fields of view of the former imagers 3518. Typically the field of view of each imager 3518 may overlap with that of at least one other imager 3518. In some embodiments, the field of view of the imager 3518 dedicated to the central portion of the bag 26 may overlap with the respective fields of view of the adjacent imagers 3518 by at least 5% (e.g. 5-10%). Including multiple imagers 3518 may allow the particulate inspection system 3500 to remain relatively compact, however, fewer (e.g. a single imager 3518) may be used by spacing the imager 3518 a greater distance from the bag 26. In such examples, the imager sensor may have a greater pixel density (e.g. 20-30 megapixel or higher). Other or additional variables such as lens focal length may be adjusted to alter the number of imagers 3518 without necessarily changing the footprint of the particulate inspection system 3500. The focal length of one or more imager 3518 included in a particulate inspection system 3500 may be adjustable in certain embodiments. Such imagers 3518 may sweep through their focal length range as image data of the bag 26 is captured to collect in focus data at across various depths within the bag 26. The bag 26 may be placed in the particulate inspection system 3500 with any text or graphics of the bag 26 adjacent the rest body 3510 so that they are not intermediate the imagers 3518 and the interior of the bag 26. Any embodiments described or shown herein as having a certain number of imagers 3518 may be modified to include a greater of fewer number of imagers 3518.

In certain examples, each of the imager(s) 3518 may be associated with at least one filter 3542. For example, imagers 3518 may each be paired with a neutral density filter in some examples. Alternatively or additionally, a polarizer 3517 may be placed in front of the lens for each imager 3518. Where polarizers 3517 are used, the polarizers 3517 for each imager 3518 may be oriented at a lensed light transmitting orientation. This orientation is an angle related to the polarization angle of the light emitted from the external illuminator 3516. This orientation may differ for each imager 3518 and may substantially block light from the external illuminator 3516 reflecting off bag material and particulate within the bag 26. The lensed light transmitting orientation may be selected to permit transmission polarized light emitted from the external illuminator 3516 and lensed or reflected by a hypothetical spherical lens within the bag 26.

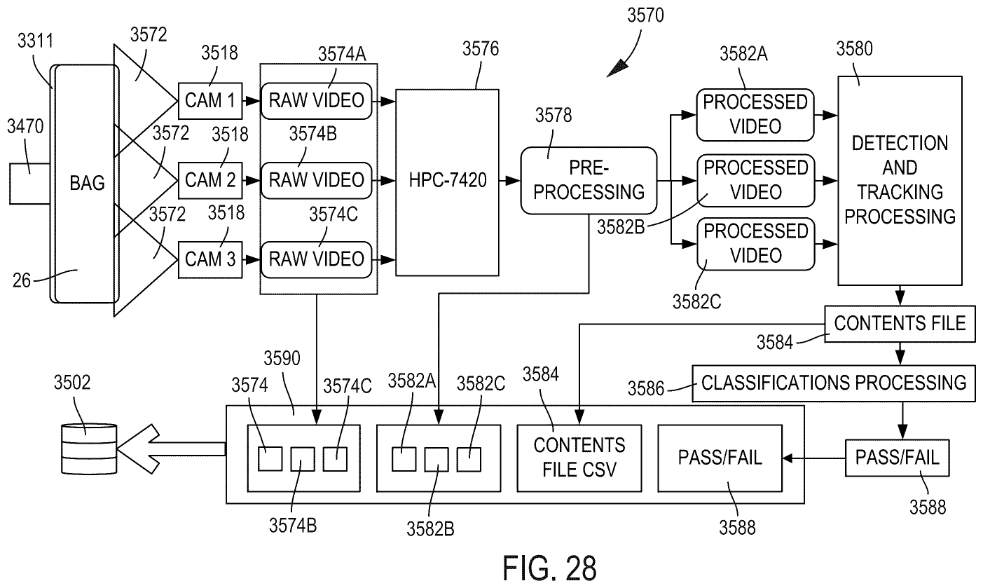

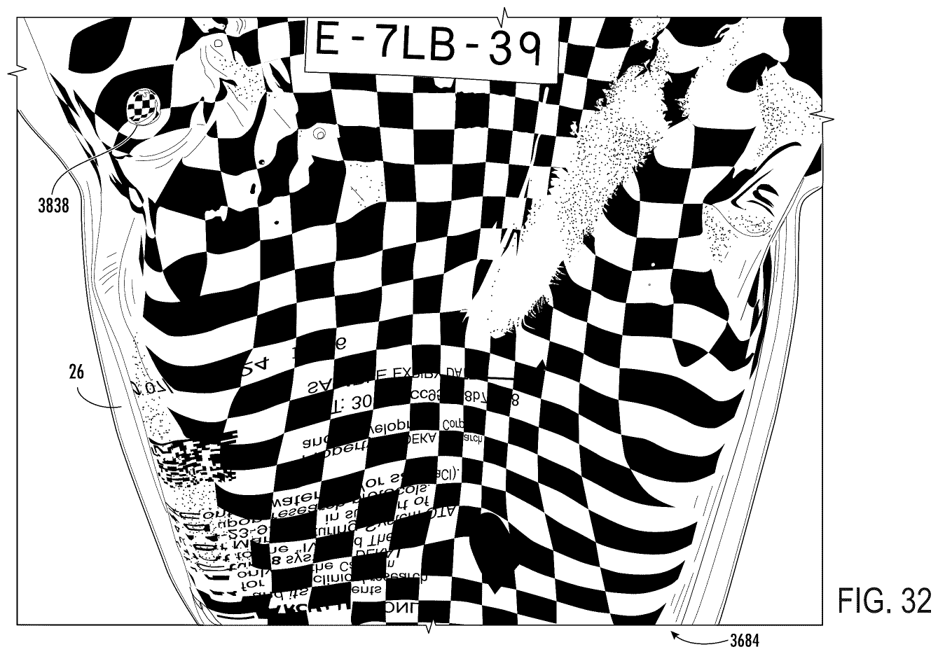

The control system 15 may command the rotary actuator 3470 to displace the bag 26 with the doors 3400A, B actuated closed upon the bag 26. In some embodiments, the control system 15 may command displacement of the bag 26 as described in relation to FIG. 17 and may mix fluid within the bag 26. The bag 26 may be returned to a starting position and the doors 3400A, B may be opened to provide a clear field of view for the imagers 3518. After a wait period has elapsed (e.g. 5-10 seconds) the control system 15 may command the imager(s) 3518 to capture frames of the bag 26. Depending on the embodiment, frames may be captured at a rate of at least 10 frames per second (though higher or lower capture rates are possible). Frames may be captured for at least 10 seconds (though capture periods greater than or less than 10 seconds are also possible). The control system 15 may process the captured frames from each imager 3518 to determine the presence of one or more contents of interest in the bag 26. In some examples, the control system 15 may include an FPGA or dedicated processor for this task. The control system 15 may create an inspection log 3590 (see, e.g., FIG. 28) which may be communicated to a database 3502 or other memory. Each inspection log 3590 may include at least one of the raw frames 3684 (see, e.g., FIG. 32) from the imagers 3518, analysis results for the frames (type of content identified, number of particles, size of particles, location of particles, etc.), information about the bag 26 (e.g. lot, unique identifier, solution type within bag 26), and a pass/fail determination for the bag 26. Where incorporated into a large parent system, the parent system may ensure that bags 26 which are determined to have failed inspection criteria are routed to a discard or quarantine destination after leaving the particulate inspection system 3500. The parent system may additionally ensure that such bags 26 are clearly labeled as rejected.

As shown in FIG. 18, a particulate inspection system 3500 may also include an indicia reader 3543 for an indicium 56 on the bag 26. The indicia reader 3543 may be an imager, barcode reader, QR code reader, RFID interrogator, NFC interrogator, etc. In certain examples, the bag 26 may include a GS-1 code as the indicium 56 and the indicia reader 3543 may be an imager. In alternative embodiments, one or more of the imagers 3518 may be utilized as the indicia reader 3543 and a dedicated indicia reader 3543 may be omitted. The control system 15 may determine at least a unique identifier for the bag 26 from data captured by the indicia reader 3543 (or one or more imager 3518) and may associate any particulate inspection records with that unique identifier when records are communication to a database 3502. In some embodiments such an indicia reader 3543 may also be disposed in other compartments of an enclosure 12. The indicia reader 3543 may determine various information from the indicium 56 (e.g. concentrate type, volume, mass, etc.) which may be used to inform dispensing of fluid into the bag 26 from a filling station, for example.

Referring now to FIG. 19A-C, a number of illustrations of an example particulate inspection system 3500 are depicted. The example particulate inspection system 3500 includes the mix assisting assembly 2500 depicted in FIGS. 14A-15B. As shown, the rest body 3510 includes a checker pattern or repeating dark and light regions 3514A, B. This pattern, though distorted by the bag 26 (see, e.g., FIG. 19B), may be visible through the bag 26 from perspective of the imagers 3518. The light and dark regions 3514A, B may be an appliqué which is placed on the rest body 3510 (e.g. the exterior surface of the rest body 3510). In alternative examples, the pattern may be generated on the rest body 3510 by, hydro dip painting, stenciling or screen printing, laser etching the pattern into a painted rest body 3510. Laser etching may also be used to generate roughness in a pattern on a rest body 3510 which is otherwise too smooth for paint to adhere. The rest body 3510 would then be painted and the paint may be removed from the unetched surface. A photoreactive coating could also be applied, exposed to light, and developed to generate the desire pattern.

The particulate inspection system 3500 may include a mount assembly 3520 to which the imager(s) 3518 may be coupled. The mount assembly 3520 may be coupled to the enclosure 12 and may ensure that the imagers 3518 are positioned at a prescribed angle (e.g. 32°) to the medial transverse plane of a bag 26. As shown, each of the imagers 3518 may be associated with a filter 3542. In various embodiments, the filter 3542 may be neutral density filter. Alternatively or additionally, a polarizer 3517 may be associated with each imager 3518 (see, e.g., FIG. 18).

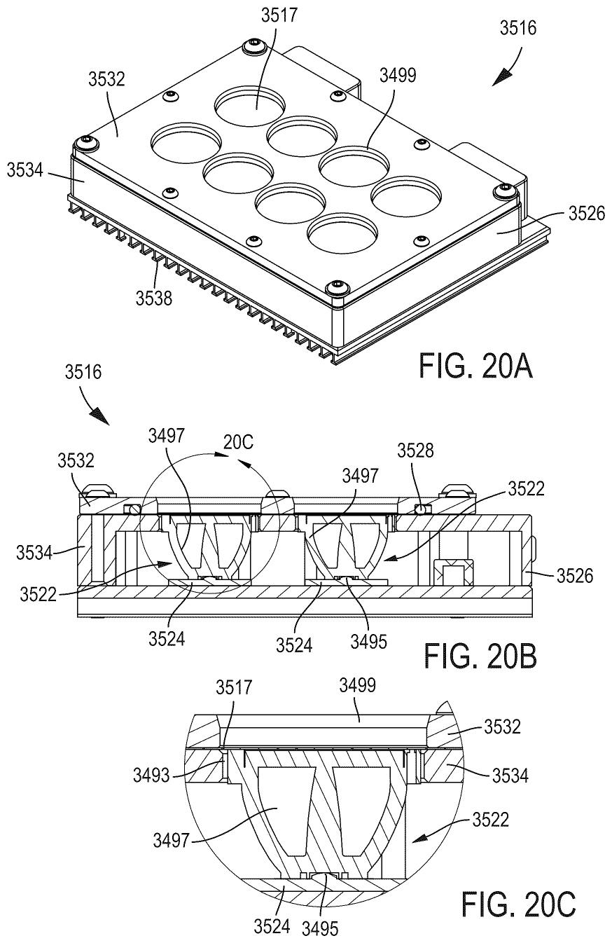

The external illuminator 3516 is also depicted in FIGS. 19A-C. Another example is depicted in isolation throughout FIGS. 20A-C. As best seen in FIG. 19C and FIG. 20B, the external illuminator 3516 may include a plurality of light emitter assemblies 3522. Each light emitter assembly 3522 may include an LED 3495 and collimating lens or narrow beam angle lens 3497. The LED's 3495 and PCB 3524 to which they are coupled may be disposed within a housing 3526. The housing 3526 may include one or a series of sealing members 3528 (e.g. o-rings) which may inhibit ingress of any liquid into the housing 3526. The sealing member(s) 3528 may be formed of a compliant material which may be compressed by a face member or plate 3532 coupled to a main section 3534 of the housing 3526. This may facilitate placement of the external illuminator 3516 under the bag 26 within a particulate inspection assembly 3500. The external illuminator 3516 may also include a polarizer 3517. The polarizer 3517 may form part of the housing 3526 and span across a light emission aperture 3536 of the housing 3526. Alternatively, multiple light emission apertures 3536 may be included and may be spanned by a polarizer 3517. This may help to leverage the polarizer 3517 as a retainer for the lenses 3497 associated with each LED 3495. The main body 3534 may include receiving apertures 3493 for an emitting end of each of the lenses 3497 which may further assist in preventing displacement of the lenses 3497. Where multiple light emission apertures 3536 are included, the apertures may include a tapered region 3499 which increases the light emission aperture 3536 width as distance from the respective lens 3497 increases. This may ensure that the light emitted from the light emitter assemblies 3522 is substantially unblocked by the housing 3526.

A heat sink 3538 may be coupled to the housing 3538 to assist in dissipating heat generated by the light emitter assemblies 3522. The heat sink 3538 (or a base intermediate the heat sink 3538 and remainder of the housing 3526) may include guides which ensure that the main portion 3534 of the housing 3526 is aligned with the light emitting assemblies 3522 during assembly. The housing 3526 may include a mount 3540 via which the external illuminator 3516 may be fixed in place relative to the imagers 3518. The mount 3540 may, for example, couple to the enclosure 12 (see, e.g., FIG. 18).

Depending on the layout of the array of individual light emitting assemblies 3522 in an external illuminator 3516, the angle of the light with respect to the height axis of the bubble 3550 may change. In the example shown in FIG. 19C for instance, the light emitting assemblies 3522 of the external illuminator 3516 are arrayed in two rows. The collimated light from these rows may be generally directed at the midplane of a bag 26 retained in the particulate inspection system 3500. To accomplish this, the light emitting assemblies 3522 may be positioned to emit light at an angle non-parallel to the midplane. For certain external illuminators 3516, the light emitting assemblies 3522 in each row of the array may be spaced an even amount from respective opposing sides of the midplane. The light emitting assemblies 3522 may emit collimated light at an angle of 15° to the midplane with each row tilted in opposite directions. In other embodiments with additional rows or different light emitting assembly 3522 array layouts, the light emitting assemblies 3522 may be angled to emit collimated light at a center point or central plane of a bag 26.

Alternatively, the narrow beam angle engendered by the lenses 3497 of each light emitting assembly 3522 (see, e.g., FIGS. 20A-C) may ensure that the entire bag 26 is bathed substantially uniformly in light from the external illuminator 3516 when the external illuminator 3516. The light emitting assemblies 3522 may not be oriented other than substantially normal to the base of the external illuminator 3516.

The light emitter assemblies 3522 may be wired in series in certain examples (though this is not necessary in all embodiments). With such an arrangement, the failure of one light emitter assembly 3522 would result in failure of the entire external illuminator 3516. This prohibits a scenario in which one or more light emitter assembly 3522 fails and is not detected. Thus, the external illuminator 3516 may be ensured to operate at only 100% output. Detection of a light emitter assembly 3522 failure would also be simplified as no light would be generated when the external illuminator 3516 is commanded on.

In some embodiments a photodetector in data communication with the control system 15 may be positioned opposite the external illuminator 3516. In the event the signal output by the photodetector is less than a threshold, the control system 15 may generate a fault. Alternatively, image data from the imagers 3518 may be utilized to detect a failure of the external illuminator 3516. The control system 15 may, for example determine an overall light intensity value for the field of view of an imager 3518 and may generate a fault if the field of view is below a darkness threshold when the external illuminator 3516 is commanded on. In some embodiments, the control system 15 may additionally or instead determine failure of the external illuminator 3516 by analyzing the color space values for image data from an imager 3518. The control system 15 may compare the image data to expected color space value ranges (e.g. RGB color space). If the external illuminator 3516 emits red light, for instance, a high R value and lower G and B values would be expected to be observed in at least portions of an image In the event that the color space values observed are unexpected (e.g. substantially uniformly low RGB values) the control system 15 may generate a fault. Alternatively, a neural net may be trained with images where the external illuminator 3516 is powered and verified to be functional. The neural net would also be trained with images taken when the external illuminator 3516 is unpowered and in a non-illuminating state. An image may periodically be supplied for neural net analysis to check for failures of the external illuminator 3516. A fault may be generated by the control system 15 in the event of a determination by the neural network that the external illuminator 3516 has failed. Where a fault is generated, the control system 15 may generate a notification for display on a user interface communicating the external illuminator 3516 failure.

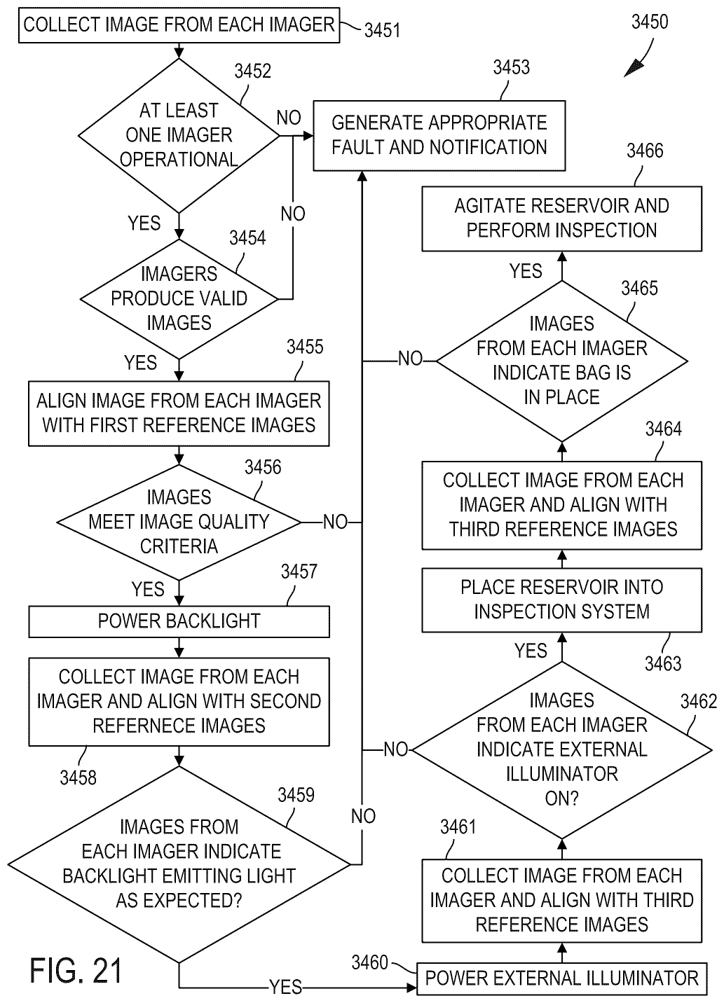

Referring now to FIG. 21, a flowchart 3450 detailing a number of exemplary actions which may be executed to verify a particulate inspection system 3500 is functioning as expected is depicted. As shown, in block 3451 the control system 15 may command image capture from each of the imagers 3518 of the particulate inspection system 3500. If, in block 3452, no imagers 3518 are operational (e.g. connections cannot be established) or if, in block 3454 an imager outputs an image which is not valid (e.g. blank or all black) a fault notification may be generated in block 3453. If, in block 3452, at least one imager 3518 is operational and operational imagers 3518 output valid images in block 3454, the functionality check may continue. Proceeding even with non-operational imagers 3518 is optional, though may be preferred as it may generate more information for service personnel tasked with troubleshooting the particulate inspection system 3500. In such instances, images may be captured from any operational imagers 3518 which are generating valid images and any image analysis may be limited to data from imagers 3518 which are determined to be functioning properly.

As shown, in block 3455, images from each imager 3518 may be aligned with respective first reference images by a processor of the control system 15. The images captured by the imagers 3518 for comparison against reference images are referred to as test images in relation to FIG. 21. The first reference images may be specific to each imager in the particulate inspection system 3500 and may be captured during set up or by service personnel on after a particulate inspection system 3500 is verified to be functioning appropriately. The image registration or alignment may be performed by matching points on the image captured by an imager 3518 to one or more corresponding reference point in the reference image for that imager 3518. This could for example be a feature (e.g. a point where corners of a number of squares of a chessboard pattern meet) of the background pattern on the rest body 3510 for example. A perspective transformation may then be performed to support a comparison of the reference images with the respective test images. If, in block 3456, the images do not meet predefined quality criteria, a fault notification may be generated in block 3453.

In various examples, the images captured in block 3455 may be compared with a pixel by pixel comparison, feature based matching, a trained neural network, or any other suitable alternative. In certain embodiments, a mean square error comparison may be conducted by the control system 15. In the event the mean square error comparison indicates a difference in excess of a predefined threshold, the quality criteria may be failed. If the threshold is exceed from the comparison of any one test image to the respective reference image, the quality criteria may be deemed failed. The output of the mean square error analysis may be in excess of the threshold in the event that the focus of the test images is unacceptable, occlusions (e.g. detritus/liquid on imager 3518 lens, or rest body 3510 including the background pattern) are present, or other distortion of the test images is present. Though examples described in relation to FIG. 21 use a mean square error analysis, other analysis may be used instead or in combination with a mean square error analysis in alternative embodiments.

If, in block 3556, the quality criteria is met, the control system 15 may command the backlight (e.g. illuminator 3512) to be powered in block 3457. With the backlight powered, an image from each imager 3518 (or operational imager 3518) may be collected in block 3458. These test images may be aligned or registered against a second set of respective reference images for each imager 3518 in block 3458 as well. The second reference images may be captured during service or setup of a particulate inspection system 3500 when the backlight is verified to be operating as expected by a technician or service personnel. If, in block 3459, analysis of the images indicates that the backlight is not emitting light as expected, a fault notification may be generated in block 3453. In certain examples, a mean square error analysis may, for example, be executed by the control system 15 using the second reference images and the test images from block 3458. If the difference output by the mean square error analysis is in excess of a threshold, the comparison may be deemed to have failed. The threshold may be breached in the event that the colors and/or light intensity in the test images does not correspond to the respective reference images. If the comparison of any one test image to the respective reference image exceeds the threshold, it may be determined the backlight is not functioning as expected.

If, in block 3459, the analysis determines that the backlight (e.g. illuminator 3512) is functioning as expected, an external illuminator 3516 may be commanded on by the control system 15 in block 3460. The backlight may typically be turned off, though need not necessarily be in all embodiments. With the external illuminator 3516 powered, an image from each imager 3518 may be collected in block 3461. These images may also be aligned with respective third reference images in block 3461. As with other reference images, the third references images may be captured by a technician or service personnel after verification that the external illuminator 3516 and any other powered lighting sources are working properly. If, in block 3462, the analysis by the control system 15 indicates the external illuminator 3516 is not emitting light, a fault may notification may be generated in block 3453. In certain examples, a mean square error analysis may, for example, be executed by the control system 15 using the third reference images and the test images from block 3461. If the difference output by the mean square error analysis is in excess of a threshold, the comparison may be deemed to have failed. The threshold may, for instance, be breached in the event that the colors and/or light intensity in the test images does not correspond to the respective reference images. If the comparison of any one test image to the respective reference image exceeds the threshold, it may be determined the external illuminator 3516 is not functioning as expected.

If, in block 3462, the comparison between the third reference images and the images captured in block 3461 indicates the external illuminator is emitting light as expected, a reservoir may be installed in the particulate inspection system 3500 in block 3463. Optionally, images may be collected from each imager 3518 after the reservoir is retained in the particulate inspection system 3500 in block 3464. These images may be utilized to verify a reservoir is properly retained in the particulate inspection system 3500. Alternatively, the doors 3400A, B of the particulate inspection system 3500 may be closed and the position of the doors 3400A, B may be monitored by the control system 15 to verify a reservoir is in place at the particulate inspection system 3500. Where reservoir installation is verified via imaging, the collected images may be aligned against reference images in block 3464 as well. The reference images used in block 3464 may be fourth reference images or the second or third reference images in some examples. Where the second reference images are used, the backlight (e.g. illuminator 3512) may be powered when the images of block 3464 are taken. Where the third reference images are used, the external illuminator 3516 may be powered when the images of block 3464 are taken. Where fourth reference images are used, both the backlight and external illuminator 3516 may be powered when the images of block 3464 are taken (no reservoir would be present). The fourth reference images may be captured by service personnel when the backlight and external illuminator 3516 are verified to be functioning as expected.

If, in block 3465, comparison of the images from block 3464 to the appropriate reference images indicates that a reservoir is absent, a fault notification may be generated in block 3453. In certain examples, a mean square error analysis may, for example, be executed by the control system 15 using the test images from block 3464 and the appropriate reference images. If the difference output by the mean square error analysis is in below a threshold, the comparison may be deemed to have failed. The threshold may be breached in the event that the test images closely match the respective reference images for each imager 3518. This would indicate that no reservoir is in place and the test images are of an empty particulate inspection system 3500. If the comparison of any one test image to the respective reference image is in breach of the threshold, it may be determined the reservoir is not properly retained.

If, in block 3465, the comparison indicates a reservoir is present, the reservoir may be agitated and inspected in block 3466. In some examples, the functionality test may be performed before each reservoir is inspected. A record of the functionality test may be included in the log file 3590 (see, e.g., FIG. 28) associated with each reservoir. In the event, that lighting components or imaging components are determined not to be functioning as expected, the reservoir may be indicated to have not passed inspection. The reservoir may be directed to a quarantine or access controlled repository where the particulate inspection system 3500 is part of a larger system. The parent system may also prohibit filling of any further reservoirs until the particulate inspection system 3500 has been serviced.

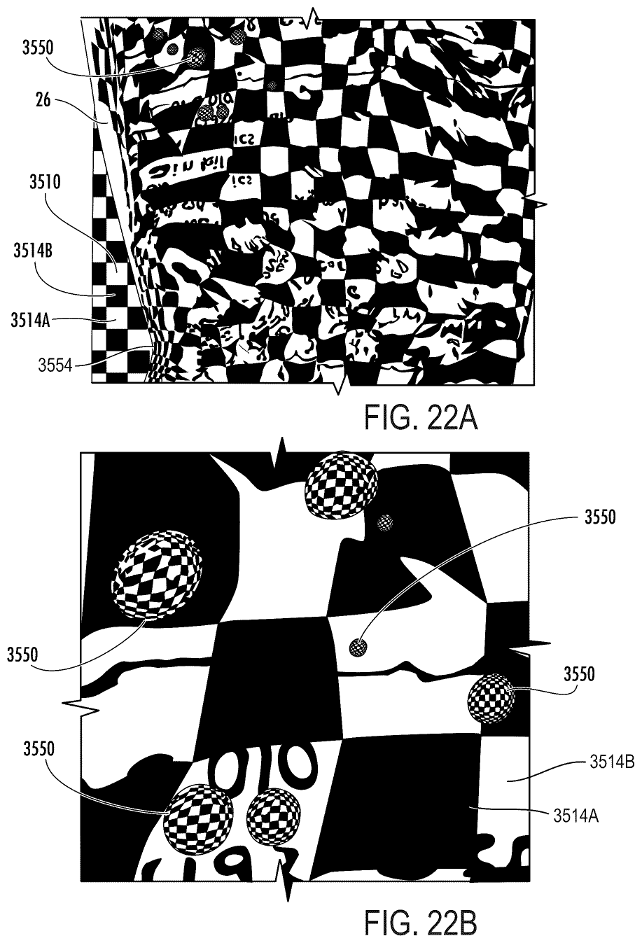

Referring now to FIGS. 22A-24D, a particle inspection system 3500 may differentiate between certain contents of interest within a bag 26. Bubbles 3550 of air or gas may be commonplace within bags 26, especially after the bag 26 has been agitated by a mix assisting assembly 2500. Bubble 3550 content should be benign and the presence of bubbles 3550 alone preferably would not trigger a bag 26 to fail inspection. The external emitter 3516, background pattern, and imager(s) 3518 may be selected, outfitted, and positioned to help ensure bubbles 3550 within the solution contained in a bag 26 are readily differentiable from other contents of interest.

Referring to FIGS. 22A-B, illustrations of portions of a bag 26 in place at a cradle 3311 of a particulate inspection system 3500 are depicted. The illustrations are representative of what would be seen from an imager 3518, but not exact reproductions of actual images. A representational frame is depicted in FIG. 22A. An enlarged portion of the representational frame of FIG. 22A is depicted in FIG. 22B. The dark and light regions 3514A, B of the rest body 3510 pattern are also visible in FIGS. 22A-B. As shown, the bag 26 may distort this pattern as light from the back light transits through the bag 26. A portion of that pattern unadulterated by passage through bag 26 is visible lateral to a crinkle or fold 3554 in the side of the bag 26 in FIG. 22A.

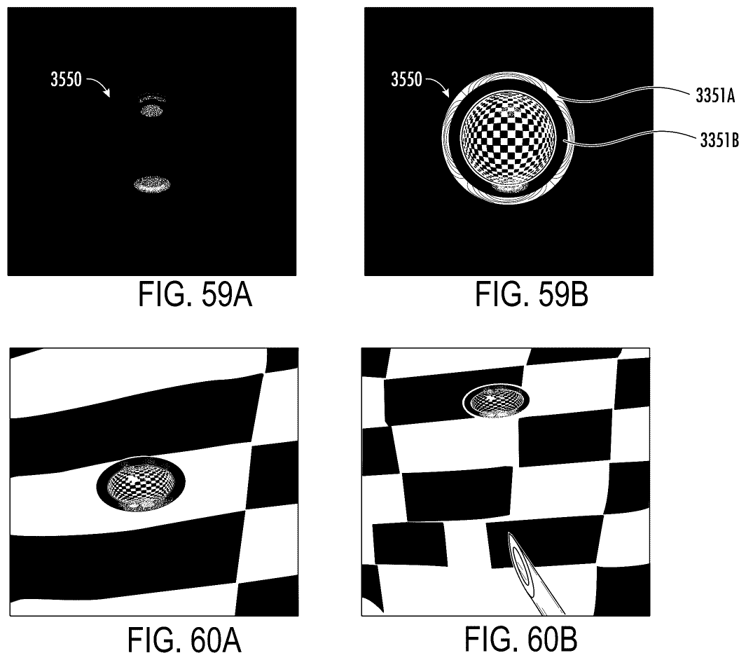

Still referring to FIGS. 22A-B, a number of large bubbles 3550 are present in the bag 26 and positioned against the wall of the bag 26 material. As shown, the optical properties of the bubbles 3550 distorts the backlight pattern in a similar manner for each of the bubbles 3550. Each of the bubbles 3550 creates a distorted version of a section of the pattern. In the example, the pattern is a chessboard pattern and a distorted checkered pattern is visible in each of the bubbles 3550. Bubbles 3550 suspended in solution may also distort the background pattern in a consistent manner (see, e.g., FIG. 59B). As the distorted versions of the background pattern are hallmarks indicating the existence of bubbles 3550, images may be analyzed for the presence of these specific patterns in order to identify the presence of bubbles 3550 within a bag 26. The control system 15 may employ a pattern recognition algorithm such as a statistical pattern recognition algorithm or syntactic pattern recognition algorithm. Template matching may for example be used to match instances of the distorted background pattern to an expected distorted background pattern template. A neural network may also be trained on images of bubbles 3550 creating the distorted checkered pattern and employed to detect the presence of such bubbles 3550 based on presence of the pattern.

The resolution of the imager 3518 capturing the image frame limits the size of bubble 3550 which may be identified by the presence of the distorted background patterns. Higher resolution imagers 3518 may detect smaller bubbles 3550 by analyzing frames for the presence of the distorted background pattern. In some embodiments, recognition of the distorted background pattern may be used to differentiate relatively large volume bubbles 3550 from other large contents of interest.

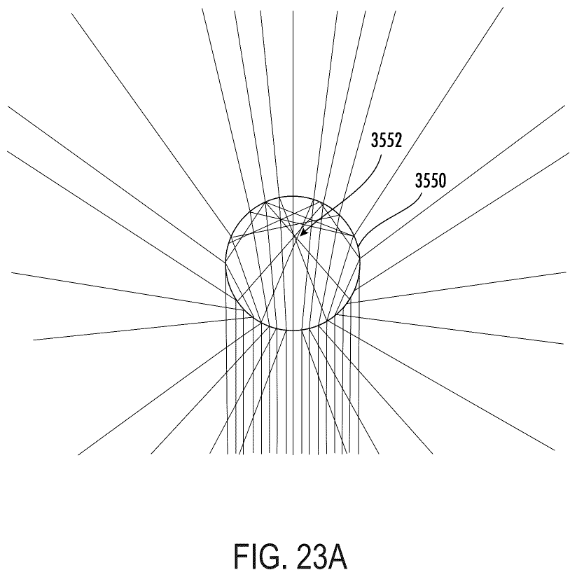

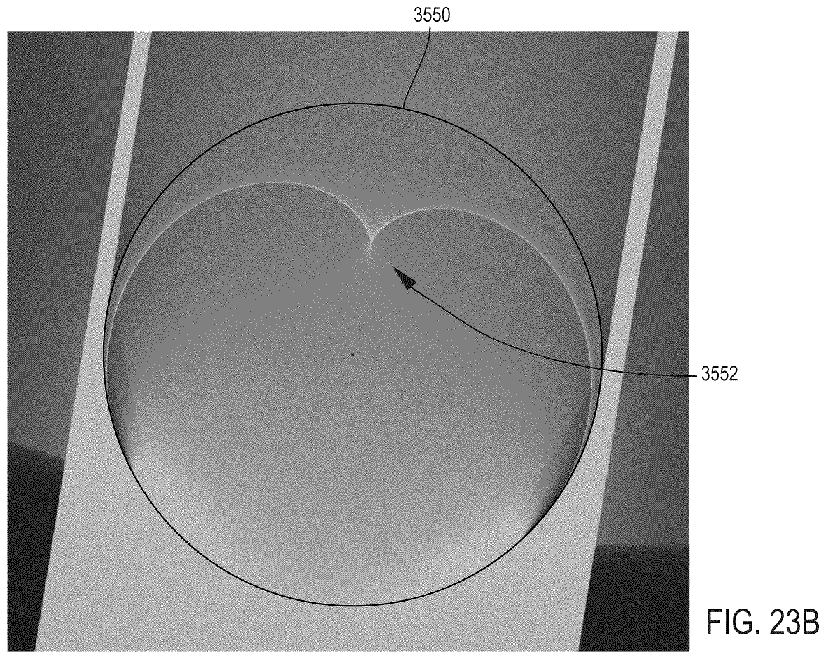

Referring now to FIGS. 23A-B, bubbles 3550 may be detected based on the manner in which they interact with light emitted from an external illuminator 3516. This may be particularly useful for identifying small bubbles 3550 and differentiating them from other similarly sized contents of interest. Representational diagrams of bubbles 3550 are depicted in FIGS. 23A-B. In general, due to the constant and evenly distributed pressures on the inside and outside of the bubbles 3550, a bubble 3550 takes on a shape which minimize its surface area. Thus, bubbles 3550 are typically spherical. Other contents of interest, however, have a very low likelihood of being as close to spherical as any bubbles 3550 present in a bag 26. The optical properties bestowed by the spherical shape of a bubble 3550 may be exploited to cause bubbles 3550 to have a consistent and highly perceptible appearance when imaged by a particulate inspection system 3500.

FIG. 23A depicts an example illustrative diagram of a bubble 3550 being illuminated with collimated light (e.g. from an external illuminator 3516 of the type described in relation to FIG. 18). The light may approach the bubble 3550 in collimated manner as shown. As mentioned elsewhere herein, the light may also be polarized. As required by Snell's Law, the path of the light will be perturbed by the bubble 3550. An illustration based on a computer simulation of the same is provided in FIG. 23B. The collimated light is directed at the bubble 3550 at an angle of 15° in FIG. 23B.

As shown, in each of FIGS. 23A-B, the light reflected within the bubble 3550 may be concentrated at a focused region 3552. Other interactions with the bubbles 3550 may further lead the bubbles 3550 to create bright points of light within the bag 26 (further elaborated in relation to FIG. 63 for example). These bright points of light may be particularly discernable when viewed from certain angles (e.g. 30-35° inclined from an axis perpendicular to the bag 26 midplane). Bright points engendered by bubbles 3350 may be significantly brighter than other contents of interest within the bag 26. The color emitted by the external illuminator 3516 may also be selected to be different from that of any potential anticipated source of particulate. Thus, not only will bubbles 3550 generate bright points within a bag 26, these points may be in a known, predefined, and controllable in color. The color preferably is selected to be a color which would not be expected from any other contents of interest with a potential to be present in the bag 26. Bright points may be generated regardless of the volume of a bubble 3550. Large volume bubbles 3550 may generate bright points of light as well as very small microbubbles (e.g. 50-100 μm diameter bubbles 3550 or smaller).

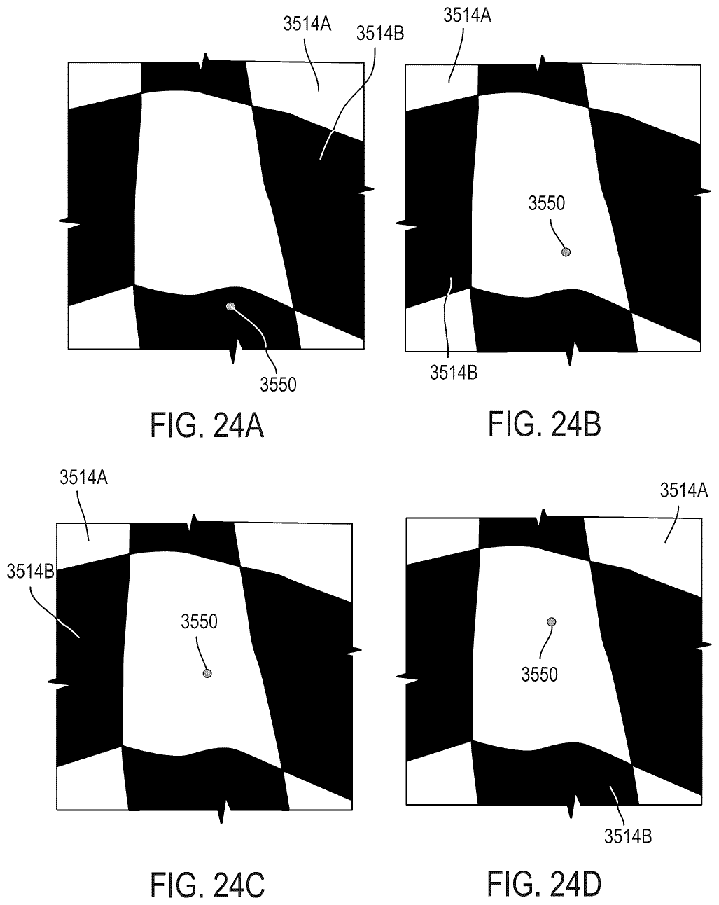

Referring now primarily to FIGS. 24A-D, a number of illustrations representative (though not exact reproductions) of portions of a bag 26 seen from an imager 3518 are depicted. Each of the illustrations depicts a bubble 3550 illuminated by an external illuminator 3516 such as that shown in FIG. 19C. The progression of FIGS. 24A-D depict the bubble 3550 displacing through the bag 26 due to, for example, buoyancy and/or agitation of the bag 26. The bubble 3550 is depicted as a grey circle which is representative of the bright light points described above. As shown, the bubble 3550 is readily distinguishable against light regions 3514A and dark regions 3514B of the background pattern.

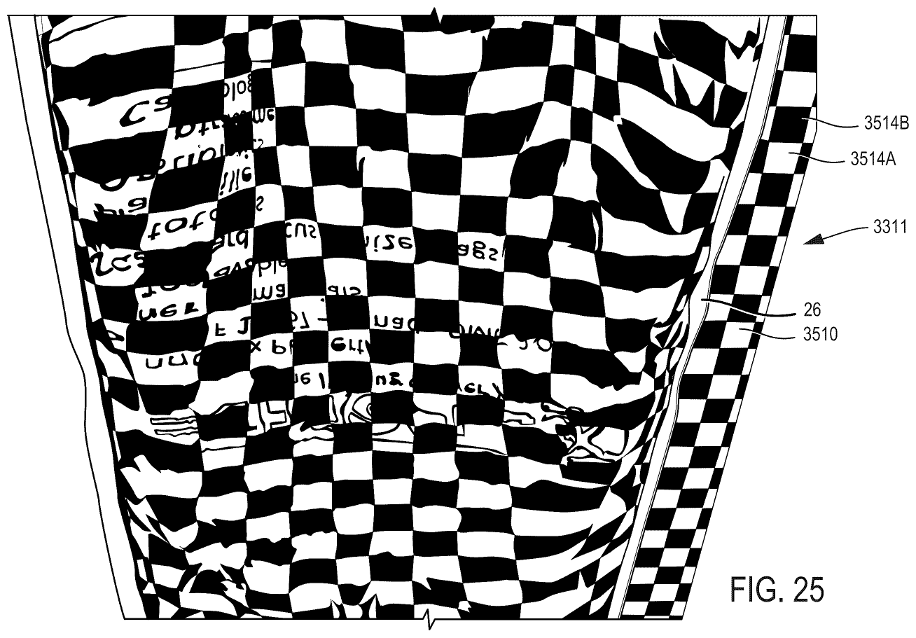

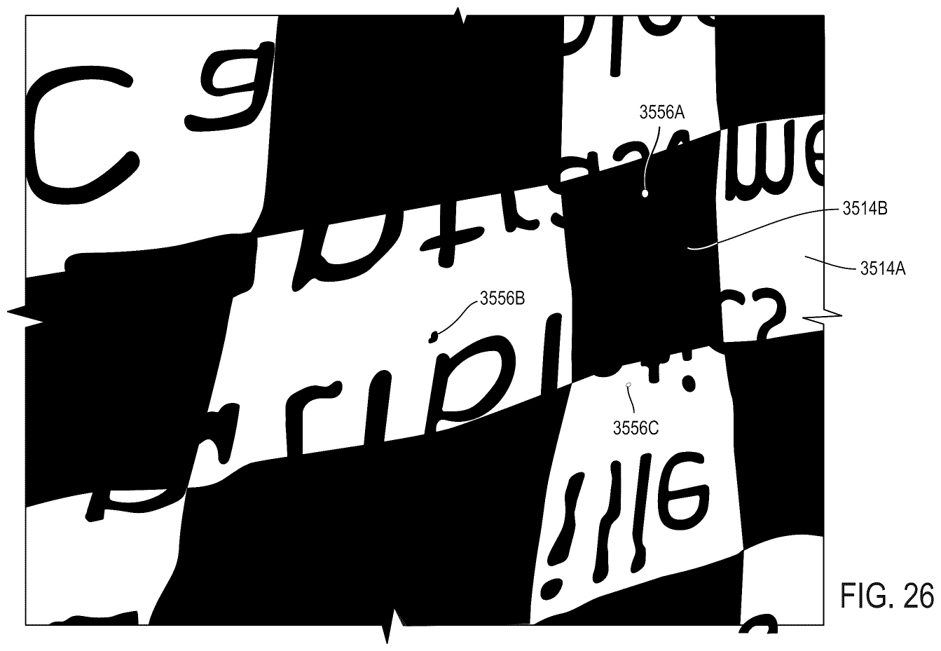

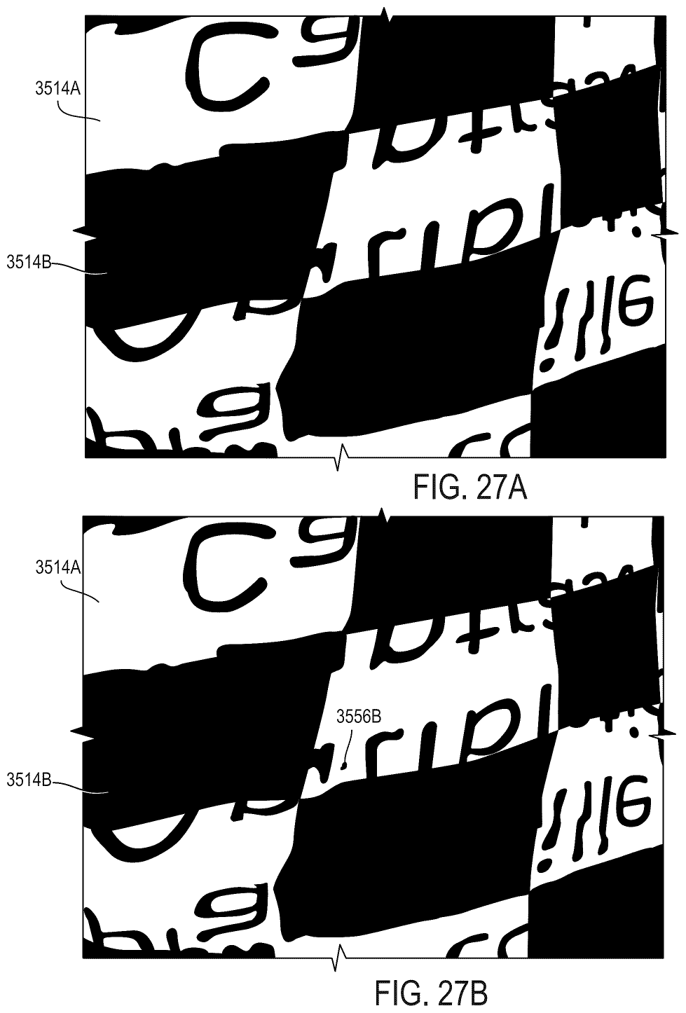

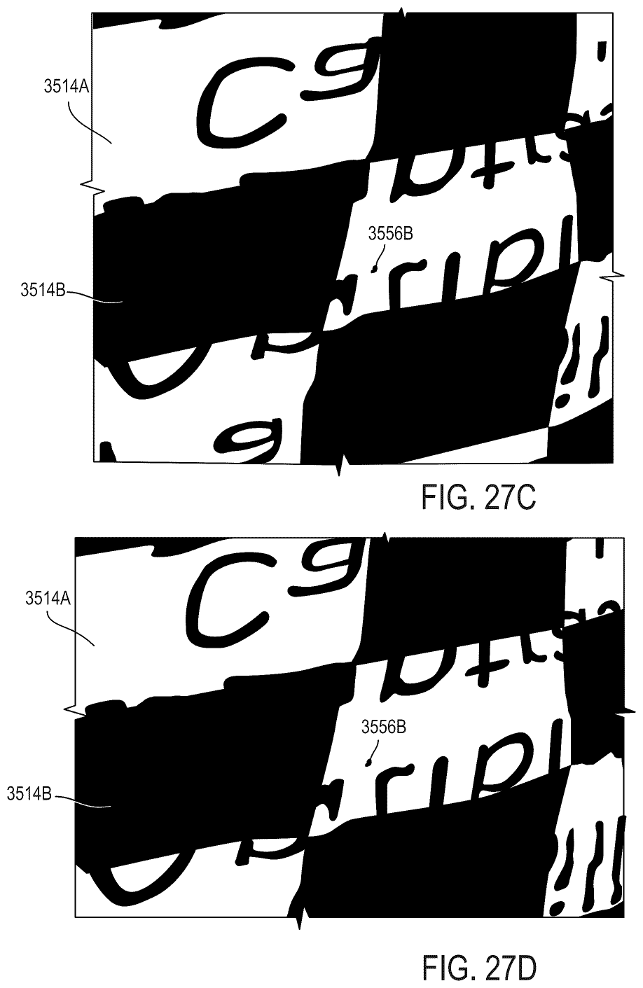

Referring to FIGS. 25-27D, further illustrations of portions of a bag 26 in place at a cradle 3311 of a particulate inspection system 3500 are depicted. The illustrations are representative of what would be seen from an imager 3518, but not exact reproductions of actual images. A representational frame is depicted in FIG. 25. An enlarged portion of the representational frame of FIG. 25 is depicted in FIG. 26. The dark and light regions 3514A, B of the rest body 3510 pattern are also visible in FIGS. 25-26. FIGS. 27A-D depict the portion of FIG. 25 shown in FIG. 26 over a period of time with an illustrative piece of particulate present (other particles in FIGS. 25-26 have been removed in FIGS. 27A-D).

Referring specifically to FIG. 25-26, a number of pieces of particulate 3556A-C of differing colors are shown. For sake of illustration white particulate 3556A, black particulate 3556B, and clear particulate 3556C are depicted. These particulate colors are selected as worst case colors in order to illustrate various advantageous aspects of example particulate inspection systems 3500 described herein. Colors of particulate from potential particulate sources in a system 10 may have greater variety and may not include the colors shown.

As shown, the backlight pattern (or a backlight otherwise controlled to generate contrast, e.g., temporally) may facilitate visualization of various pieces of particulate 3556A-C as they displace within the bag 26. This may be true regardless of the color of a particular piece of particulate 3556A-C. Prior to capturing frames with the imagers 3518 of the particulate inspection system 3500, the rotary actuator 3470 (see, e.g., FIG. 19C) may be commanded to rotate the cradle 3311 and bag 26 through an agitation sequence or motion profile. The agitation sequence may, in some examples, be a series of rotations as described in relation to FIG. 17. In such embodiments, mixing of the fluid within the bag 26 may also serve to agitate the fluid within the bag 26 for the particulate inspection system 3500. In alternative embodiments, the agitation sequence may occur at a different time point (e.g. subsequent) mixing. In such embodiments, the agitation sequence may include serial rotations between pairs of angular position. The rotation rate commanded by the control system 15 to the rotary actuator 3470 may be lower than the rotation rate used during mixing. The rotation rate used for agitation may be a function of the frame capture rate of the imagers 3518.

As best shown in FIG. 27A-D, the agitation sequence may cause particulate within the bag 26 to displace. As the cradle 3311 includes a pattern of repeating dark and light regions 3514A, B, a particle may displace across contrasting regions as a result of the agitation. Thus, particles which may be difficult to visualize on one background color may transit over a second background color against which they may be more easily seen. Providing contrast by temporally adjusting an illuminator 3512 may similarly facilitate visualization of particles which are difficult to see against certain background colors. FIGS. 27A-D depict a black particle 3556B for sake of example. The black particle 3556B is not visible in FIG. 27A as it is in alignment with a dark region 3514B of the background pattern. Due to the agitation of the fluid within the bag 26, the black particle 3556B transits over light regions 3514A of the background pattern as time progresses. As an imager 3518 captures additional frames with the black particle 3556B over light regions of the background pattern, the black particle 3556B is readily distinguishable. This is best shown in FIGS. 27B-D. The size of the light and dark regions 3514A, B may typically be made relatively small to assist in maximizing the number of transitions across light and dark regions 3514A, B for a given particle. A white or light colored particle would complimentarily be readily distinguishable as it moves over dark regions 3514B of the background pattern.

With respect to substantially transparent particles, movement of the particle may similar assist in visualizing the particle within the reservoir or bag 26. The transparent particle will distort light passing through it. The reflected or refracted light would be able to be visualized against the background pattern as the particle displaces. Transparent particles will also distort the background pattern. Again, this may assist in allowing visualization of such particles.

Referring now to FIG. 28, an example data flow diagram 3570 of a particulate inspection system 3500 is depicted. As shown, a number of imagers 3518 are included and each have a field of view 3572 which includes a portion of a bag 26 in place at a cradle 3311. Each of the fields of view 3572 overlaps with that of at least one other imager 3518. The imagers 3518 may capture raw video streams 3574A, B, C. The raw video streams 3574A, B, C may be committed to a log file 3590 associated with the subject bag 26 via a unique identifier for the bag 26. The log file 3590 may also include other identifying information. For example, the log file 3590 may include a date and timestamp. Other potential information may include a unique identifier specific to the individual parent system in which the particulate detection system 3500 is included or for the particulate detection system 3500 itself. The type of solution in the bag 26 may be included in some embodiments. Software version for the parent system, particulate detection system 3500, and/or neural network version (further described elsewhere herein), any calibration data or settings, user ID information, etc. may also be included in a log file 3590.

In the example, each of the raw video streams 3574A, B, C is also communicated to a server 3576. In other examples, the server 3576 may be replaced with a microprocessor, graphical processing unit, application specific integrated circuit, programmable logic controller, complex programmable logic device, field programmable gate array, or other dedicated processing hardware, and combinations thereof. Dedicated hardware such as an FPGA may be desirable as it may speed processing and remove burden from other processors in the control system 15. Communication may be via a wireless protocol or communication may be wired (e.g. via USB 3.2) depending on the embodiment. The server 3576 may, in some examples, be remote (e.g. cloud) and may receive data from a plurality of client particulate inspection systems 3500 (or their parent systems). Distributed computing environments may also be used.

The server 3576 may fed the raw video stream 3574A, B, C from each imager 3518 into a respective queue. Frames may be extracted from the respective queues and fed into a respective thread. Frames from each thread may then be analyzed for the presence of particles. Analysis on all frames corresponding to a subject bag 26 may be required to be completed before data from another bag 26 is analyzed. In some embodiments, frames of a given bag 26 from each imager 3518 may be processed in series. In other examples, threads of frames from each imager 3518 for a given bag 26 may be processed concurrently.

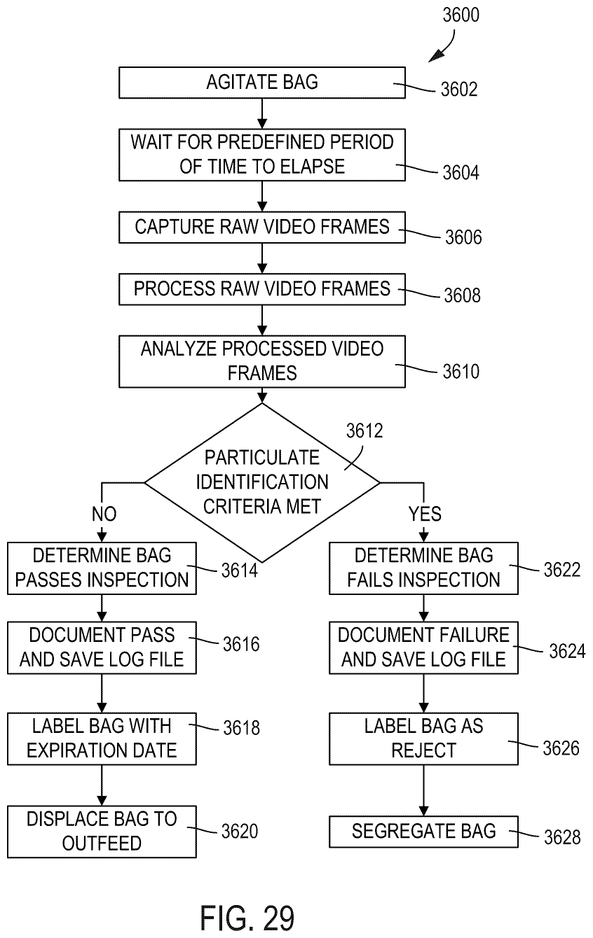

Analysis of the video may include pre-processing 3578 and detections and tracking processing 3580 (see, e.g., FIGS. 29-31, FIG. 41). The pre-processing 3578 may include background subtraction, foreground isolation, filtering with a foreground mask, kernel morphological transformations such as dilations, erosions, openings, closings, morphological gradients, image noise reduction filtering, etc. The pre-processing 3578 may generate processed video streams 3582A, B, C from the raw video streams 3574A, B, C. The processed video streams 3582A, B, C may also be committed to the log file 3590. The pre-processing may also include an analysis of the image data to identify features captured in the frames which are to be determined invalid candidates for further analysis. For example, the pre-processing may identify features which are defined by more than a predetermined number of constituent pixels and flag them as invalid.

The detection and tracking processing 3580 may utilize the processed video streams 3582A, B, C and generate a contents analysis file 3584 (e.g. CSV). The contents analysis file 3584 may specify displacement paths or tracks for any regions of interest within frames that are identified in the processed video streams 3582A, B, C. Image regions of interest may be pixels or pixel collections that appear to displace across the background from frame to frame. Tracks may be determined by comparing adjacent or temporally proximate frames for a pixel cluster that has moved between the frames. If, for instance, the temporally proximate frame includes a pixel cluster within a threshold distance from a pixel cluster's location in the earlier frame, it may be identified as a displacing region of interest. A prediction could be made (e.g. via a Kalman filter) for a pixel cluster's position in a temporally proximate frame and if a pixel cluster in the proximate frame sufficiently corresponds with the prediction, a displacing region of interest may be identified or declared. Clusters of pixels which move across the background may be documented in the contents analysis file 3584, though the contents analysis file 3584 may also include data on single pixel size outputs that displace in this manner as well. The displacement path may be a set of coordinates for each frame in the processed video stream 3582A, B, C. Snapshots of the regions of interest in each frame may also be placed in the contents analysis file 3584. The content analysis file 3584 may also be committed to the log file 3590.

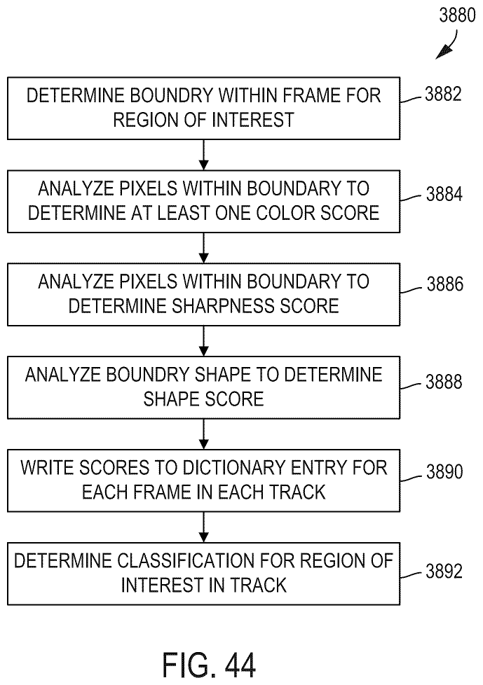

Classifications processing 3586 may be performed to analyze the contents analysis file 3854 to determine if the subject bag 26 includes contents of a first classification, second classification, and potentially further classifications (further described in relation to FIG. 44). The classifications may determine if any of the image regions interest meet one or more undesirable content identification criteria. Regions of interest may be assigned classifications by the classification processing 3586. Classifications may be binary (e.g. benign or undesirable, particle or bubble, first classification or other, bubble or not bubble) or may include additional information. For example, certain embodiments may classify a region of interest in a genus and potentially a species. For example, the classification processing may identify a region of interest as a particle (genus) of undissolved concentrate (species). Alternatively, the classifications processing could class a region of interest as a particle (genus) and a fiber, hair, or elongate particle (species), or particle of a particular color (species). Various other genus and species type classifications are also possible. The above are merely non-limiting examples. An inspection pass/fail indication 3588 may be generated based on the classifications and output by the classification processing 3586. In the event that no image regions of interest are detected or that none of the image regions of interest meet an undesirable content identification criteria, the classifications processing 3586 may determine the bag 26 is acceptable. In the event that one or more region of interest meets an undesirable content identification criterion, the classifications processing 3586 may determine the bag 26 fails inspection. The classifications processing 3586 may in some embodiments be performed by a convolutional neural network which receives as input a cropped image of every region or pixel cluster of interest associated with a given track. Alternatively or additionally, regions of interest may be analyzed by the classifications processing 3586 and assigned various scores (color, blurriness, shape, size, brightness, trajectory, etc.). The scores may be analyzed to generate a classification of the region of interest.

Where a convolutional neural network is used, the convolutional neural network may be trained using bags 26 with known contents. The system used to fill the bags 26 may be reviewed to determine potential sources of particulate. Potential sources of particulate from the bag 26 (or other reservoir) manufacturing process or bag 26 itself may also be identified. Identified potential particulates may then be deliberately introduced into, included in, or created within training bags 26 which are then placed in a particulate inspection system 3500. The training particulate would be representative (size, aspect ratio, density, buoyancy, solubility, color, roughness, material type, etc.) of any particulate encountered during true usage scenarios.

Additionally, the training bags 26 may be generated to entirely or substantially remove confounding contents of interest. For example, in bags 26 dosed with training particulate, the bags 26 may be filled with fluid which has been filtered and degassed. For example, the fluid may be degassed via heating, ultrasonic degassing, vacuum, or some combination thereof. Additionally, for any training bags 26 including particulate, a syringe or similar implement may be introduced into the bag 26 once filled. Remaining air in the bag 26 is withdrawn with the syringe to eliminate or substantially eliminate any headspace in the bag 26. The bag 26 may be utilized for training relatively quickly to prevent any transmission of gas through bag 26 material.

For undissolved concentrate, bags 26 may be dosed with concentrate to their saturation point and additional concentrate may then be added. Alternatively, water vapor may be allowed or encouraged to evaporate through the bag 26 material until, for example, at least some concentrate in a brine contained with the bag 26 falls out of solution. Degassed, filtered water may be added to the bags 26. The bags 26 may be agitated, though not agitated aggressively enough to redissolve the crystallized concentrate and quickly utilized for training to prevent the crystallized concentrate from fully dissolving. Alternatively or for additional training bags 26, a filtered and degassed saturated solution of the same variety as the concentrate already in the bag 26 may be introduced to prevent or mitigate redissolution of the crystallized concentrate.

Training bags 26 containing bubbles but no particulate may also be generated. Two bags 26 may for example be placed into communication with one another. A fluid path from a first to the second bag may be present and may include a check valve upstream of a filter. Fluid from the first bag may be passed through the filter to the second bag. A fluid path from the second bag to the first bag may be present and may include a check valve upstream of a filter. Fluid may be passed from the second bag to the first bag. This may be repeated a number of times. The check valves may ensure flow through each filter is unidirectional. Thus, any particulate in the bags 26 may be collected by the filters and removed from the liquid in the bags 26. Integrity of the filters may be subsequently verified (e.g. with a bubble point test). This may allow a bag 26 which contains air bubbles, but substantially no particulate, to be produced for training.

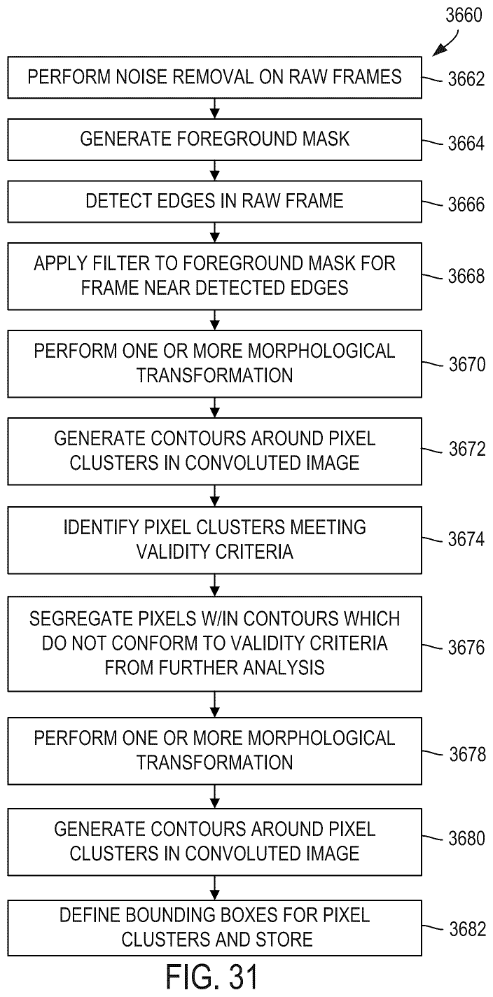

The training bags 26 may be displaced in a mix assisting assembly 2500 according to the displacement profile used during normal operation and images may be captured of the bag 26. Illumination, imager 3518 placement, lens filters, etc. may be kept consistent with true usage conditions. Data from such images may be used as ground truth datasets to train the convolutional neural network. Regions of interest corresponding to the known bag 26 contents may be detected (see, e.g., FIG. 31) and input to the convolutional neural network as training data.

A pass or fail indication from the classifications processing 3568 may be written to the log file 3590. In the event the bag 26 fails inspection it may be marked as rejected and placed in a quarantine or discard location after removal from the particulate inspection system 3500. The log file 3590 may be communicated to a database 3589 for remote review and analysis.

In some embodiments, a notification may be generated in the event a fail indication is determined. The notification may be generated for display on a graphical user interface, for example. Depending on the classification, a suggestion may also be made (and potentially displayed in the notification) as to a likely source (or sources) for the particulate. For instance, a processor of the control system 15 may check the classification against a list of potential sources of particulate. The list could include, for example, score sets for respective potential sources determined using bags deliberately dosed with particulate from those sources (or representative of those sources). Scores closely matching those of an item in the list may cause the suggestion to identify that specific item (or a set of best matches from the list). If the particulate is, for example, classified as green and the only potential green source of particulate is present in the manufacturing environment for the reservoir, the suggestion may indicate the source is likely the manufacturing environment (or a specific component therein).

In the event that excessive computation time has been required for analysis, a notification may similarly be generated for display. For example, if the control system 15 takes greater than a threshold amount of time to detect, track, and classify regions of interest in a bag 26, a fault may be triggered and a notification to that effect may be communicated to a user via a graphical user interface. In the event that the number of regions of interest determined to be particles is higher than a threshold, the control system 15 may trigger a fault. A notification may, for example, be generated by the control system 15 indicating an upstream problem in a parent system for producing and packaging fluids or manufacturing environment may be present.

Referring now to FIG. 29, a flowchart 3600 depicting a number of example actions which may be executed to detect the presence of contents of interest within a bag 26 is depicted. A particulate inspection system 3500 may agitate a bag 26 (or other reservoir) in block 3602. This may be accomplished by issuing commands from a control system 15 to a rotary actuator 3470 of a particulate inspection system 3500 (see, e.g., FIG. 17). A dwell time may then elapse in block 3604. The dwell time may vary from embodiment to embodiment, but may be 3-10 seconds (e.g. 5 seconds) in some examples. Once the dwell time has elapsed, the control system 15 may command capture of video frames by each of imager 3518 of the particulate inspection system 3500 in block 3606. At least 70 (e.g. 100) frames from each imager 3518 may be captured over some predetermined period of time for each bag 26. In other examples, less than 70 frames may be collected. Each raw video stream 3574A, B, C may, for example, be captured at a rate of 10 frames per second. Raw video streams 3574A, B, C captured from each imager 3518 for each bag 26 may be ten seconds in length. In block 3608, the raw video streams may be processed by a processor of the control system 15. The processed video frames may be analyzed by a processor of the control system in block 3610.

If, in block 3612, particulate identification criteria are not met, it may be determined that the bag 26 has passed inspection in block 3614. The control system 15 may document the pass and save a log file in block 3616. The control system 15, in block 3618, may command the bag 26 be collected from the particulate inspection system 3500 and displaced to a labeling or marking assembly (e.g. with a gantry or robotic arm). The bag 26 may also be labeled with an indication that the bag 26 is acceptable (e.g. embossed with an expiration date) in block 3618. In block 3620, the bag 26 may be displaced to an outfeed from a parent system.

If, in block 3612, particulate identification criteria are met, it may be determined that the bag 26 has failed inspection in block 3622. The control system 15 may document the failure and save a log file in block 3624. The control system 15, in block 3636, may command the bag 26 be collected from the particulate inspection system 3500 and displaced to a labeling or marking assembly. The bag 26 may also be labeled with an indication that the bag 26 is not to be used (e.g. embossed with the word “REJECT”) in block 3626. In block 3628, the bag 26 may be segregated within the system 10. The bag 26 may be placed in a quarantine or discard receptacle, for instance. Quarantine and labeling may, in some implementations be accomplished as described in U.S. Pat. No. 11,980,587, issued May 14, 2025, entitled Systems, Methods, and Apparatuses for Producing and Packaging Fluids (Attorney Docket No. 00101.00325.AA697).

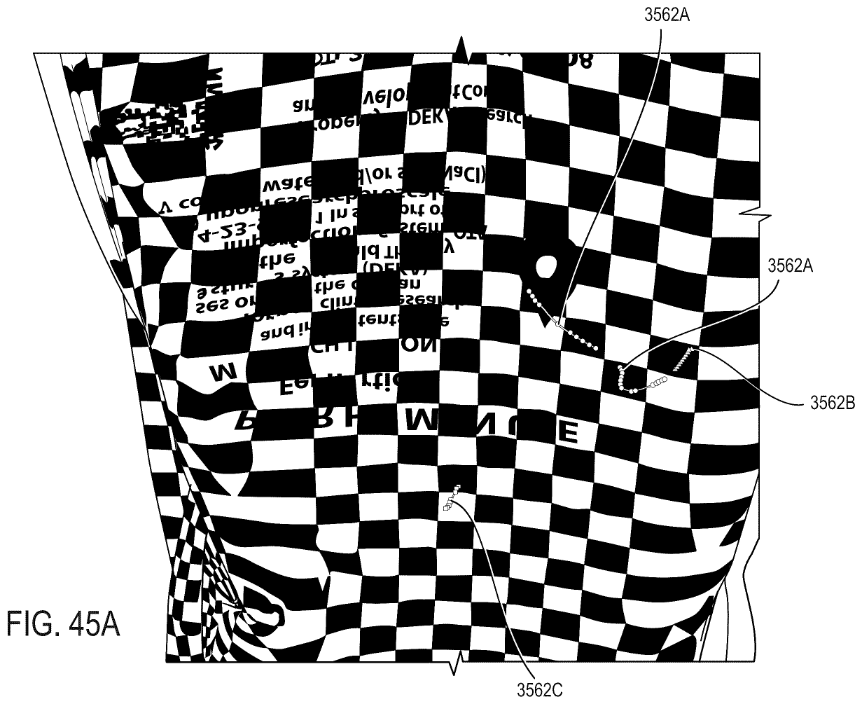

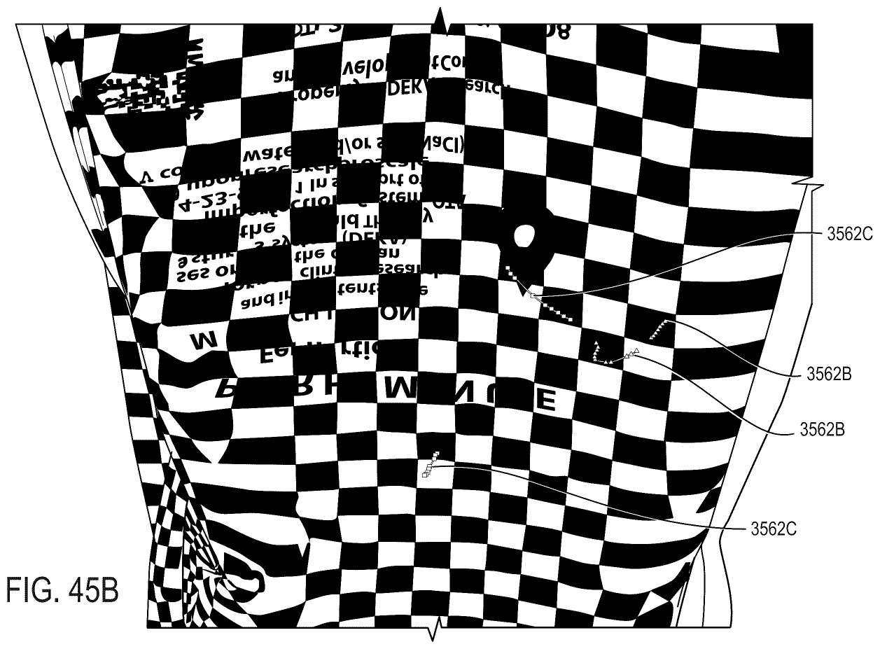

The log files 3590 saved in blocks 3616, 3624 may include any of the raw video, processed video(s), frames thereof, an analysis summary, pass/fail determination, bag 26 or lot identifying information, information about the contents of the bag 26, a unique identifier for the bag 26, etc. In some embodiments, the processed video streams 3582A, B, C may be scaled down before addition to the log file 3590. In other embodiments, a trail may be generated for each region of interest identified and may grow over each frame to show the historical position of the region of interest over the course of the video. The trail may be generated by interpolating between the coordinates of each specific region of interest over adjacent frames. In other examples, a summary may be generated for inclusion in the log 3590. In some examples, the summary may be an annotated version of a frame (e.g. the final captured frame) from each imager 3518. The annotated frame (see, e.g., FIG. 45B) may include a trail of any contents of interest identified in the video frames and a classification of the content of interest (e.g. bubble or particle, bubble/not bubble). Any inspection log files 3590 may be communicated to a database for storage and remote review. Summaries may be or include a data file (e.g. CSV) giving coordinates for each tracked content of interest and respective classification determinations.

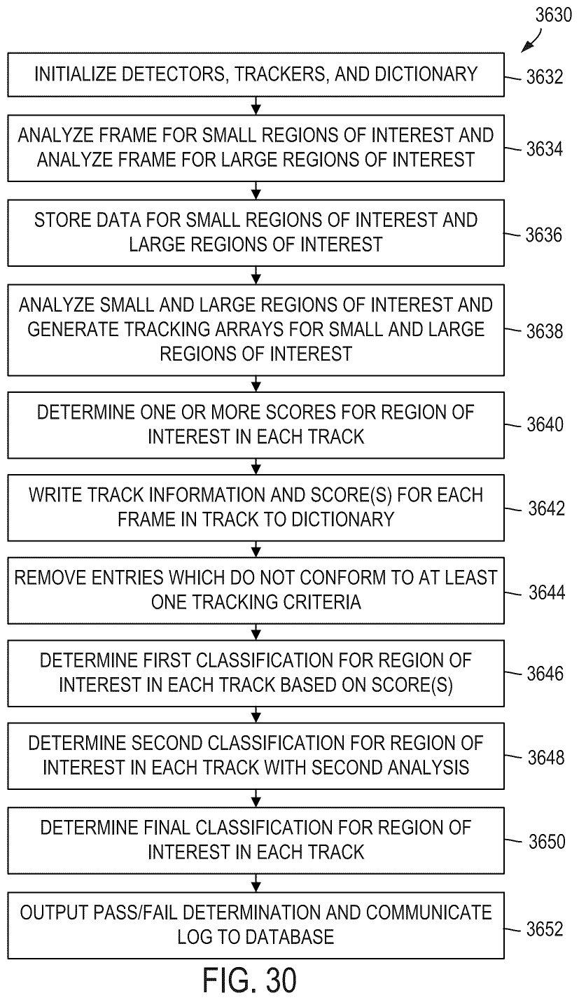

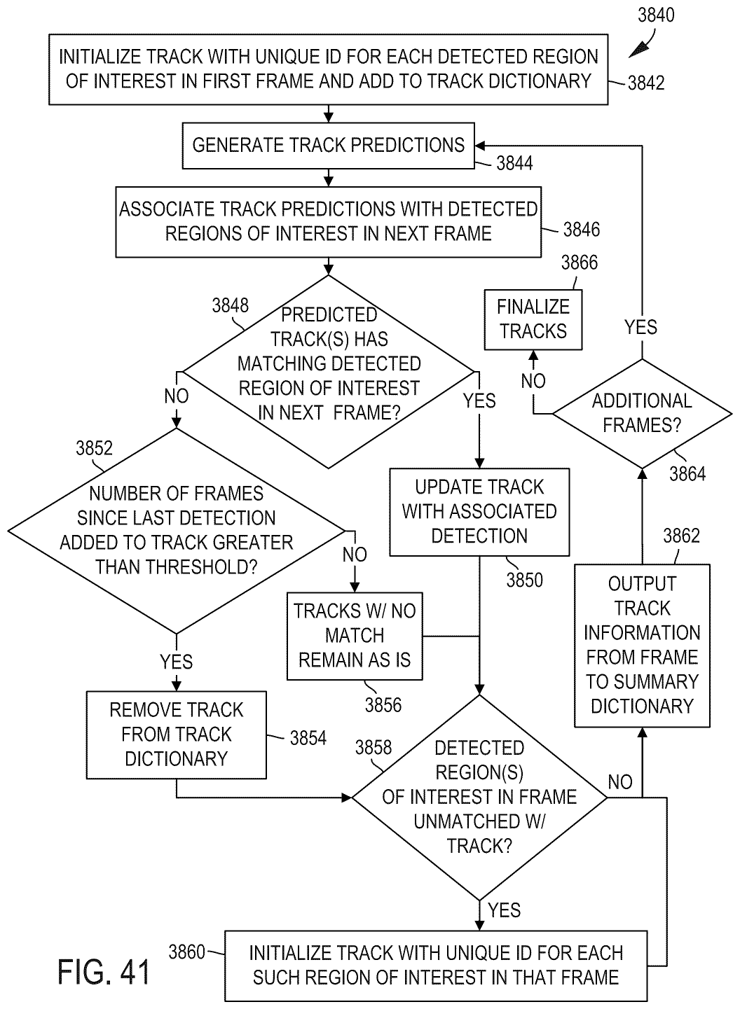

Referring now to FIG. 30, another flowchart 3630 detailing a number of exemplary actions which may be executed to detect and track contents of interest within a bag 26 over a number of video frames is depicted. In block 3632, a processor of the control system 15 may initialize a number of detectors (see, e.g., FIG. 31), trackers (see, e.g., FIG. 41), and at least one dictionary. In some embodiments, a detection dictionary (e.g. an array for storing detection information), a tracking dictionary (an array for storing track information during tracking analysis), and a tracking record dictionary may be initialized. The tracking record dictionary may be a data repository including a selection of information relating to tracks for regions of interest in frames captured by imagers 3518 of a particulate inspection system 3500. Coordinates within a frame, various scores such as any of those discussed herein (see, e.g., FIG. 44), and a clipped snapshot of any regions of interest for each frame may for example be stored in the tracking record dictionary.

In block 3634, each frame from each imager 3518 may be analyzed for small regions of interest and large regions of interest. Frames may be analyzed sequentially and may be analyzed one imager 3518 at a time. Small regions of interest may be for example be those having an area of 5,000 pixels or less and large regions of interest may be those having an arca greater than 5,000 pixels. Regions beyond a certain size (e.g. area greater than 20,000 pixels) may be ignored in certain embodiments. Alternatively each frame may be analyzed for additional region of interest size ranges (e.g. extra-large having an area greater than 20,000 pixels). Detection of regions of interest is further described in relation to FIG. 31.

Each identified region of interest for each frame may be assigned a bounding box 3834 (see, e.g. FIG. 39A-B) which delineates the region of interest. The bounding boxes 3834 may be stored (e.g. written to an array) in block 3636. Small and larger regions of interest may be analyzed by a processor of the control system 15 in block 3638 to identify displacement tracks for individual regions of interest across a plurality of frames. Tracking of regions of interest is further described in relation to FIG. 41. Any suitable tracking algorithm may be utilized. For example, a multiple object tracking algorithm may be utilized. In some implementations a SORT (Simple Object Realtime Tracking) algorithm, MHT (multiple hypothesis Tracking) algorithm, JPDA (Joint Probabilistic Data Association) algorithm, etc. may be used.

In block 3640, a processor of the control system 15 may analyze the tracks and assign one or more scores to each identified track. Scoring is further described in relation to FIG. 44. The track information and associated scores may be saved (e.g. written to a tracking record dictionary) in block 3642.

In some embodiments, and as shown in block 3644, entries which do not conform to one or more tracking criteria may be removed. A track may be required to persist over at least some predefined number of frames. A region of interest generating the track may also or instead be required to displace at least some predefined distance (e.g. number of pixels). Tracks which do not meet these criteria may be removed from further consideration. They may be deleted from a tracking record dictionary or separate dictionary used to facilitate region of interest tracking in certain example embodiments.

A first classification for the region of interest associated with each track may be determined based on the at least one score in block 3646. At least one additional classification for the region of interest associated with each track may be determined in block 3648. Each additional classification may be determined with a different variety of analysis. In certain embodiments, a convolutional neural network trained on images of bags 26 with known contents may be used. In block 3650, a final classification may be determined for the region of interest associated with each track. The final classification may be determined based on at least one of the first classification and additional classifications. Alternatively, data (e.g. scores) used to determine any of the non-final classifications may be analyzed to arrive at the final classification. Certain data from one analysis may be weighted heavily or be controlling on the classification determination. For example, in the event a shape score indicates the region of interest is highly elongate, the region of interest has a very low likelihood of being representative of a bubble. The shape score may become controlling or strongly predominate toward as identifying the region of interest as a non-bubble when it is indicative of an aspect ratio beyond a predetermined threshold.

An acceptability determination (e.g. pass/fail) may be made based on the final classification in block 3652. A log 3590 may also be generated in block 3652 and communicated to a database 3502 (see, e.g., FIG. 28) or other electronic storage medium. Electronic memories described herein may be any of a variety of types including optical memories (Blu-Ray, DVD, CD, etc.), USB sticks, flash drives, solid state storage devices, hard drives or other magnetic storage media, secure digital or SD card, database, EEPROM, etc.

The log 3590 (see, e.g., FIG. 28) may include a CSV of track identities and frame by frame coordinates for each region of interest. The log 3590 may also include raw images generated by the imager 3518. At least one set of processed frames (frames after background subtraction, kernel convolution, etc.) generated from the raw images may be included in a log 3590. The log 3590 may also include an annotated version of the final frame collected by each imager 3518 with the tracks overlaid thereon. Lines interpolating the travel path between frame by frame coordinates for each region of interest may be drawn over the image. The final classification may also be indicated on the annotated image. In some embodiments, the frames from each imager 3518 may be stitched together to form a single image of the entire reservoir. An annotated image (see, e.g., FIG. 45B) may be included in the log 3590.

Referring now to FIG. 31, a flowchart 3660 depicting a number of example actions which may be executed to detect regions of interest within images of a reservoir is depicted. Throughout the description of FIG. 31, reference is made to FIGS. 32-40B which provide representations of exemplary images relating to aspects of the flowchart 3660. The representations are not exact reproductions of actual images and are provided for illustrative purposes. Certain features may be removed and others adjusted or exaggerated to facilitate illustration and reproducibility. Where image is used herein to describe the content of a particular drawing, it should be understood the “images” are representative illustrations.

As shown, in block 3662, a processor of the control system 15 may receive each raw frame 3684 and denoise the frames. A representation of a raw frame 3684 is shown in FIG. 32. To denoise the image, values assigned to pixels may, for instance, be adjusted based on an average or weighted average of surrounding pixels. Some specific examples implement Gaussian smoothing for denoising raw frames 3648

The frames from each imager 3518 may be analyzed by a processor of the control system 15 to generate a foreground mask in block 3664. This may be accomplished in any suitable manner. In various examples, a Gaussian mixture based background/foreground segmentation algorithm may be used. An Open CV MOG or MOG2 algorithm may be utilized for instance. In other embodiments, statistical background image estimation combined with per pixel Bayesian segmentation may be used. Deep neural networks, robust principal component analysis (RPCA) segmentation, Dynamic RPCA, etc. are non-limiting alternative options.

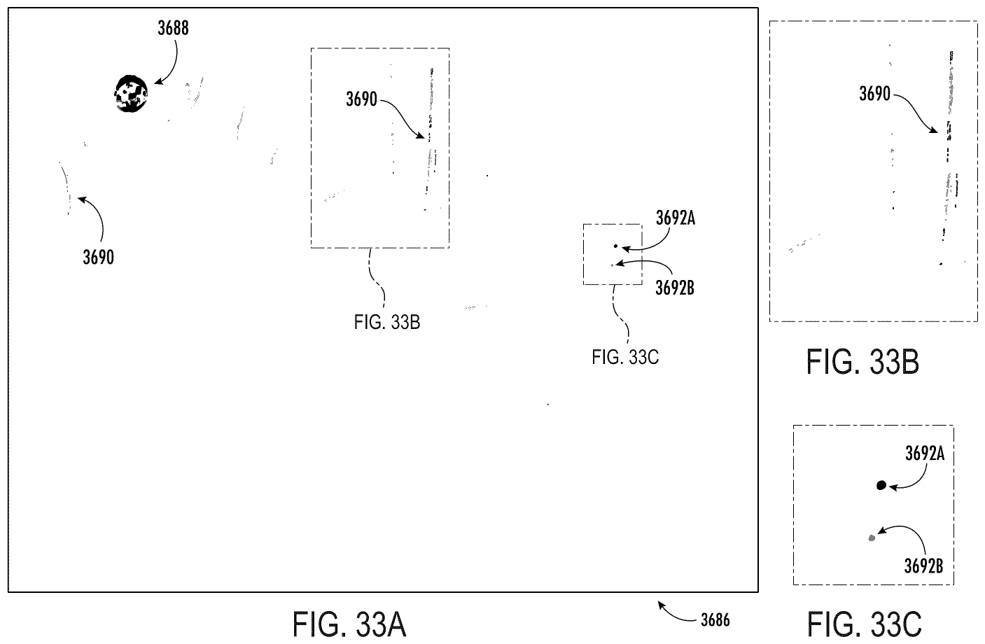

Referring now also to FIGS. 33A-C, a foreground segmented image 3686 representative of what would be generated from the raw frame 3684 depicted in FIG. 32 is depicted. As shown, the dark and light regions 3514A, B of the background pattern on the rest panel 3510 have been removed in the foreground segmented image 3686. Additionally, various edges, crinkles, and folds in the bag 26 or other stationary features are removed. Text on the bag, scratches, logos, appliqués, etc. would be removed. The representative foreground segmented image 3686 provided in FIG. 33A includes a first collection of pixels 3688 in a region corresponding to the location of a relatively large bubble 3838 in FIG. 32. The bubble 3838 is against the wall of the bag and the collection of pixels 3688 may be present due to slight movement of this bubble 3838 and/or settling of the bag 26 over time. As best shown in FIG. 33B, an enlarged portion of the indicated region in FIG. 33A, there may be some small remnants 3690 of the background in the foreground segmented image 3686. These remnants 3690 could be representative of features of the bag 26 or the pattern on the rest body 3510 for instance. The background remnants 3690 may be present, due to subtle settling or shifting movement(s) of the bag 26 over the progression of raw frames 3684 taken by an imager 3518. The representational background segmented image 3686 also includes some putative regions of interest 3692A, B as shown in FIG. 33C (an enlarged portion of the indicated region of FIG. 33A).

Again referring to FIG. 31, in block 3666, a processor of the control system 15 may analyze the raw frames 3684 and detect edges within these frames 3684. An edge detection algorithm may be used for this purpose. Any suitable algorithm may be used. In some example a multi-stage algorithm such as a Canny edge detection based algorithm may be used. Gradient based (e.g. Sobel, Scharr, Prewitt, Roberts Cross edge detection) or second order derivative type (e.g. Laplacian) edge detection algorithms may be used. An edge image may be created from each of the raw frames 3684. Binary thresholding may be applied to ensure the edge image is a black and white only output.

A filter may be applied to the foreground segmented image 3686 created from each raw frame 3684 near the detected edges in the respective raw frame 3684 in block 3668. Applying a filter to the foreground segmented image 3686 may assist in filtering out edges detected due to settling motion of flexible reservoirs (e.g. bags 26) over the imaging window. In some embodiments, a morphological transformation may be applied to the edge image. For example a dilation type kernel convolution may be applied. This may thicken the detected edges to better accommodate shifting of certain types of reservoirs over time. The convolution used may depend on the size of region of interest the detection analysis is tuned for. In some examples, a smaller dilation may be used when detecting large regions of interest. In some examples, no morphological transformation may be used for detecting regions of interest in certain size ranges (e.g. large regions and extra-large regions).

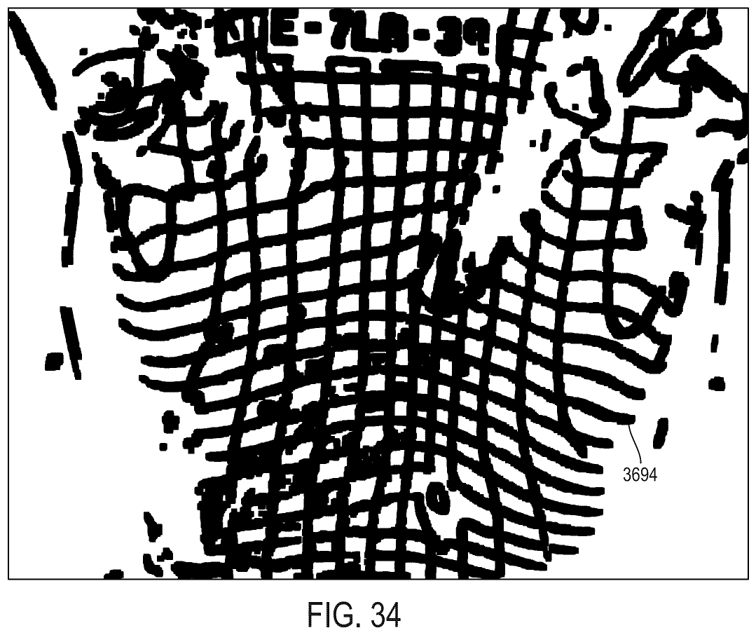

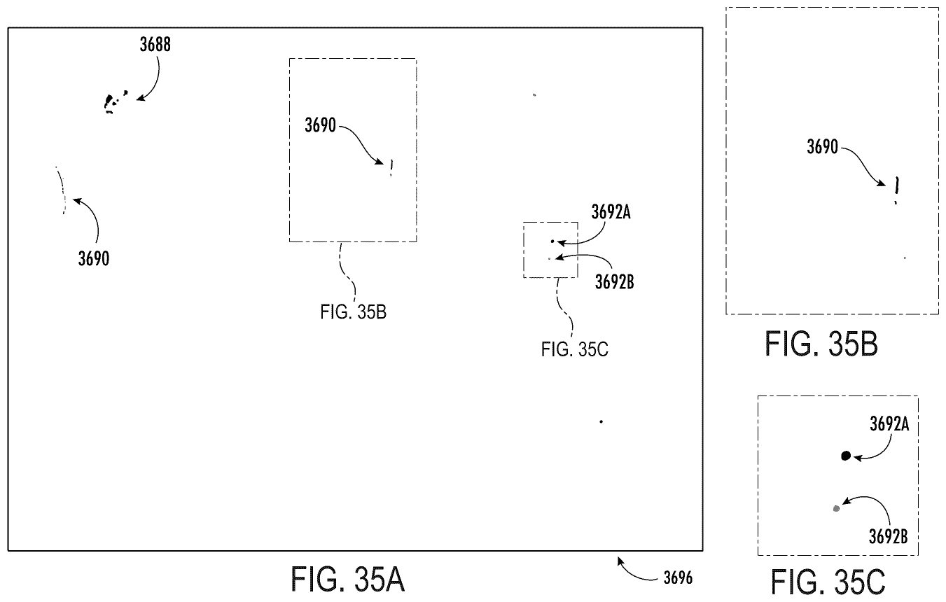

FIG. 34 depicts a visualization of detected edge regions 3694 (after dilation) in the raw frame 3684 represented by FIG. 32. Regions of the raw frame 3684 where edges are determined to be present are shown in black in FIG. 34. A representative near edge filtered foreground segmented image 3696 is depicted in FIG. 35A. The near edge filtered foreground segmented image 3696 is exemplary of what would be generated from the foreground segmented image 3686 of FIG. 33A. As shown best in FIG. 35B, the background remnants 3690 may be entirely removed or substantially diminished after applying filtering in regions of the image near detected edges. The example putative regions of interest 3692A, B may be relatively unaltered. Such near edge filtering may not be utilized for detections of certain size regions of interest (e.g. large/extra large).

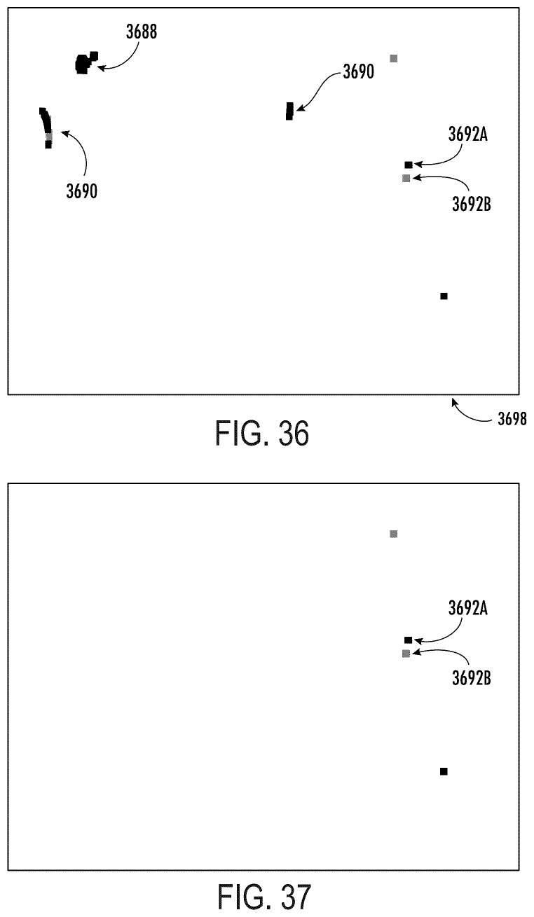

Referring again to FIG. 31, in block 3670 one or more morphological transformation is performed on the near edge filtered segmented foreground image 3696 corresponding to each raw frame 3684 by a processor of the control system 15. In certain implementations a kernel convolution such as a dilation may be performed. A representation of an exemplary dilated image 3698 generated from the near edge filtered foreground segmented image 3696 of FIG. 35A is depicted in FIG. 36. A dilation may make collections of pixels ultimately corresponding to large features in the raw image 3684 become one or more amalgamated regions of pixels. As shown, the collection of pixels 3688 corresponding to the large bubble 3838 in the raw frame 3684 (see FIG. 32) is represented by a contiguous region of white pixels in the dilated image 3698. The background remnants 3690 are also dilated into continuous streaks of pixels. The regions of pixels defining the putative regions of interest 3692A, B are enlarged, but no so much as to merge.

With reference to FIG. 31, contours may be generated around pixel clusters of interest in the convoluted image in block 3672. Contours may be generated via a contour tracing algorithm such as a pixel or border following algorithm, a vertex following algorithm, or a run-data based algorithm. In some examples, a square tracing algorithm, Moore-Neighbor Tracing (e.g. with a Jacob's stopping criterion) algorithm, radial sweeping algorithm, Pavlidis algorithm may be used. The contours may be compared against one or more validity criteria in block 3674. The validity criteria may be a range for the total number of pixels, or area in pixels within the identified contour. If more than a threshold number of pixels are included within the bounds of a contour, the contour may be determined to be invalid. Alternatively or additionally, if less than a threshold number of pixels are included within the bounds of a contour, the contour may be determined to be invalid. This may for example be true when detecting larger regions of interest. Pixels in the dilated image 3698 corresponding to large features in the raw image 3684 and background remnants 3690 may typically be characterized as invalid due to their merger and resultant size at least when detecting small regions of interest. Pixels within contours which do not conform to the validity criteria may be removed from further analysis in block 3676. A representation of the dilated image 3698 of FIG. 36 with pixel clusters in invalid contours (those larger than some preset threshold in the example) removed is shown in FIG. 37.

With reference to FIG. 36 and FIG. 37, in some examples, a statistical analysis for each pixel cluster of interest may be determined by the control system 15. A confidence interval may, for example, be calculated for each pixel cluster of interest which is representative of the strength of the signal for that pixel cluster. Clusters with a lower signal to noise ratio (e.g. after Gaussian smoothing) would have a wider confidence interval. For sake of example, black is used to depict clusters of interest with tight confidence intervals and gray is used to depict clusters of interest with confidence intervals wider than some predefined threshold. In some examples, if a confidence interval calculation is wider than some predefined threshold, the pixel cluster of interest may be flagged as invalid and removed from further analysis. Alternatively, a confidence interval may be determined for each non-background pixel in an image (see, e.g., FIG. 33A) and carried through the rest of the detection analysis.

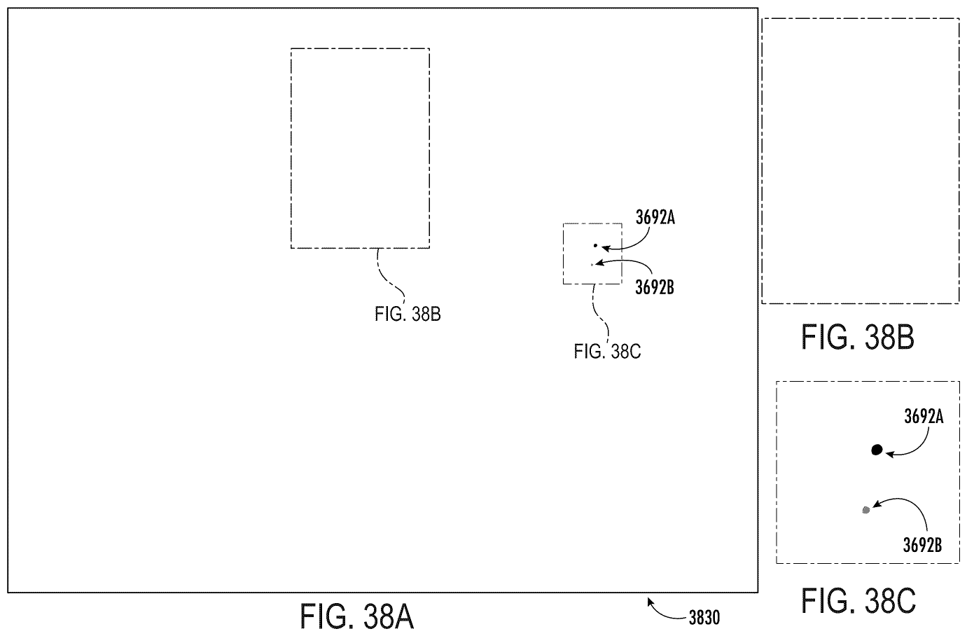

In block 3678, one or more morphological transformation different from that in block 3670 may be performed on the near edge filtered foreground segmented image 3696 created from each raw frame 3684. A kernel convolution such as an erosion may be used. A representation of an exemplary eroded image 3830 generated from the near edge filtered foreground segmented image 3696 of FIG. 35A is depicted in FIG. 38A. The erosion may return or substantially return pixel clusters to their original size and shape. At the same time, the erosion may also cause the pixel clusters have a sharper appearance. As shown, the eroded image 3830 includes the putative regions of interest 3692A, B (best shown in FIG. 38C) but the collection of pixels 3688 is absent. Additionally, as illustrated by FIG. 38B, the background remnants 3690 are absent as well.

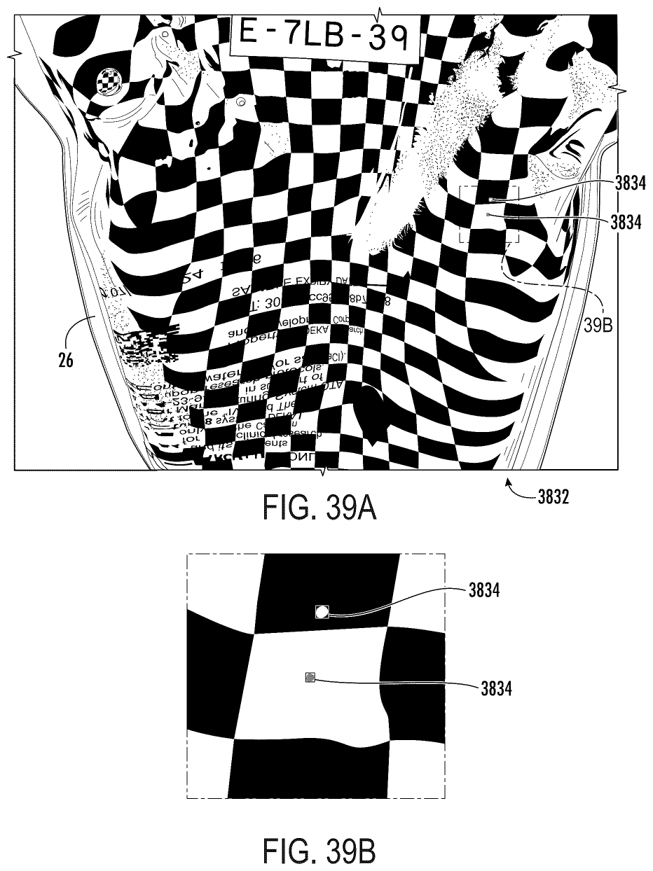

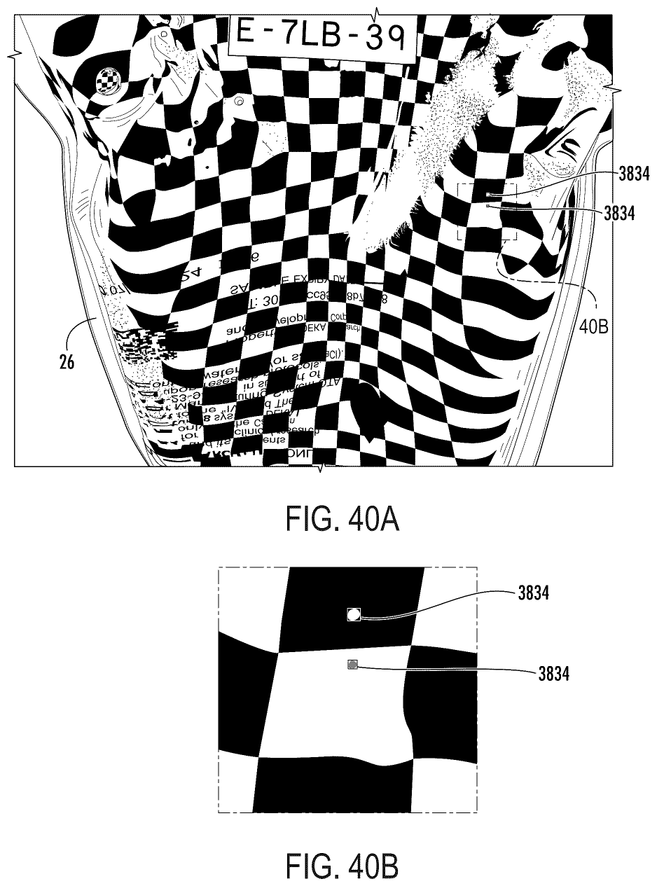

Contours may be generated around pixel clusters of interest in the convoluted image in block 3680. Though reference is made to pixel clusters, it should be understood that a single pixel could also provoke generation of a contour and a single pixel should be understood to be encompassed by the term pixel cluster. Bounding boxes 3834 for each pixel cluster of interest may be generated and stored (e.g. in an array) in block 3682. Bounding boxes 3834 may be defined based on a pixel at a corner of the bounding box 3834 along with a height and width in pixels for that bounding box 3834. A representative annotated image 3832 with bounding boxes 3834 superimposed thereon is depicted in FIG. 39A. An enlarged view of the portion of the image 3832 including bounding boxes 3834 is shown in FIG. 39B. Once pixel clusters are given a bounding box 3834 they transition from putative regions of interest 3692A, B to detected regions of interest. The detected regions of interest may be analyzed to determine respective tracks and classifications if warranted (e.g. are associated with tracks which satisfy validity criteria). As shown in FIGS. 40A-B, the bounding boxes 3834 associated with regions of interest may displace frame to frame. The annotated image 3836 in FIGS. 40A-B is generated from an analysis of a raw frame 3684 taken a number of frames after that shown in FIG. 32. The detected regions of interest may be used in multiple classifications. They may be scored (see, e.g., FIG. 44) and may be input to a neural net trained to classify regions of interest in specific embodiments.

Depending on the embodiment, each frame may be processed and analyzed to detect regions of interest a plurality of times. A first time may detect regions of interest which are within a first size range. A second time may detect regions of interest which are in a second size range different from the first (and so on). The pixel count within the area representing the region of interest may define the size of the region of interest. Different tuning may be used for processing and analysis of each frame depending on the size range of regions of interest to be identified. For example, the manner in which the foreground mask is generated or filtered may differ. Kernel convolutions may be more or less aggressive depending on the size range and/or the kernel convolutions may be bigger or small depending on the size range. For the first size range, the size of the kernel may be 20×20 pixels for example. For the second, larger size range, the size of the kernel may be 40×40 pixels. The validity criteria for the analysis of contours in block 3672 may also vary depending on the size range of regions of interest to be identified. The invalid contour criteria (see block 3674) may be arranged to ensure gross motion (e.g. of the bag 26 or large bubbles) is filtered out and not considered for later analysis. That is, regions of interest having a size beyond some predefined threshold may form invalid contours regardless of the size region of interest a particular detection analysis is tuned for. Regions of interest in the first size range may be deemed invalid when contours are generated and analyzed for detections of regions of interest in the second size range.

Referring now to FIG. 41, a flowchart 3840 is depicted detailing a number of example actions which may be executed to track regions of interest within images of a reservoir from an imager 3518. In some embodiments, as with detections, separate analyses may be conducted for regions of interest falling within each respective size range. The same actions may be executed, but would be differently tuned to cater to the respective size ranges for the detected regions of interest.

As shown, in block 3842, a track may be initialized and assigned a unique identifier for each detected region of interest in a first frame. A coordinate and velocity may be determined for each region of interest and associated with the respective track identity. The information may be stored in a tracking dictionary. This information may also be passed to a separate tracking record dictionary in block 3842. In block 3844, the position of the region of interest in the next frame may be predicted for each track. A Bayesian filter such as a Kalman filter may be utilized in certain examples. The track predictions may be associated with matching detected regions of interest identified in the next frame in block 3846. An assignment algorithm may be utilized for this purpose. For example, a bipartite matching algorithm or specifically the Hungarian Method algorithm may be utilized. The total cost used for such an assignment algorithm may be measured in, but is not limited to being measured in, distance (e.g. in pixels), intersection over union, mean square error, or some combination thereof.

If, in block 3848, a predicted track has a matching detection in the following frame, the respective track may be updated with the associated detection in block 3850. The coordinates for the region of interest and the velocity of the region of interest for the frame may be added to the track. If a predicted track has no matching detection in block 3848, a check may be made as to the number of frames since a detection matching that track has been found.

In the event that the number of frames since the last detection assigned to a track is greater than a predefined threshold in block 3852, the track may be removed from the tracking dictionary in block 3854. This may decrease resource demand as predictions for this track would no longer be determined. It may also increase the rapidity with which frames may be analyzed for tracking purposes. Where the particulate inspection system 3500 is included in a fluid production and packaging system, this may facilitate an increase in system throughput. If, in block 3852, the number of frames is less than the threshold, the track may be retained in the tracking dictionary, but not updated in block 3856.

If, in block 3858, there are detected regions of interest in the current frame which have not been matched with a track, respective new tracks with unique identities may be initialized in block 3860. In the event a frame includes a detection for a region of interest which generated a previously removed track (see block 3854), a new track would be initialized and the region of interest would be tracked under a different unique identity. Optionally, a processor of the control system 15 may assign removed tracks to tracks initialized in later frames. Thus separate tracks may be assembled into reconnected tracks under a single identity in certain embodiments. For example, if a detection several frames after a track has been terminated is within a predicted position range of the end of the terminated track, the terminated track may be connected to the track associated with that subsequent detection. In some examples, this may be done only if there are no other detections initializing new tracks in the predicted position range from the terminated track for a preset number of frames.

After initializing any new tracks in block 3860, or if no detections requiring new track initializations are present in block 3858, the track information for the frame may be output to the tracking record dictionary in block 3862. Thus, though tracks may be removed from the tracking dictionary, the tracking record dictionary would contain a record of all trackings with unique identifiers and frame by frame detection information for each track.

If, in block 3864, there are no additional frames, the tracks may be finalized in block 3866. If, in block 3864, additional frames are present, the flowchart 3840 returns to block 3844.

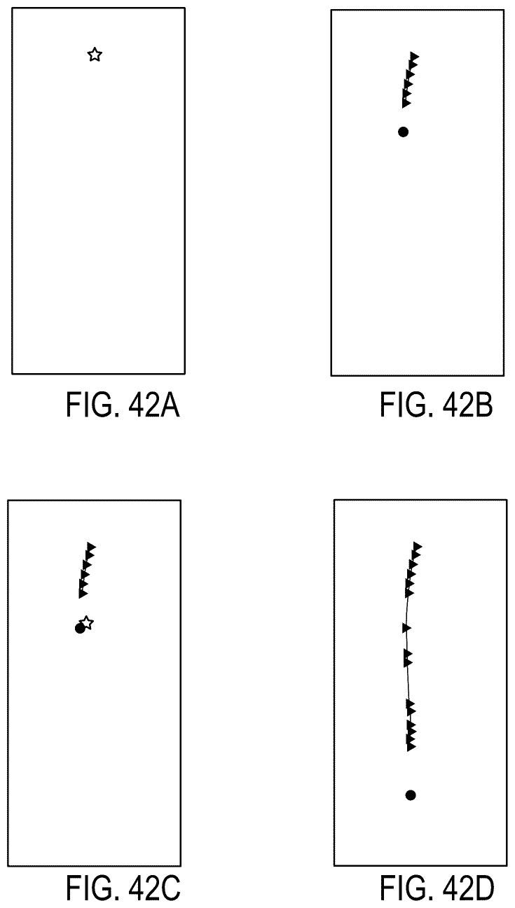

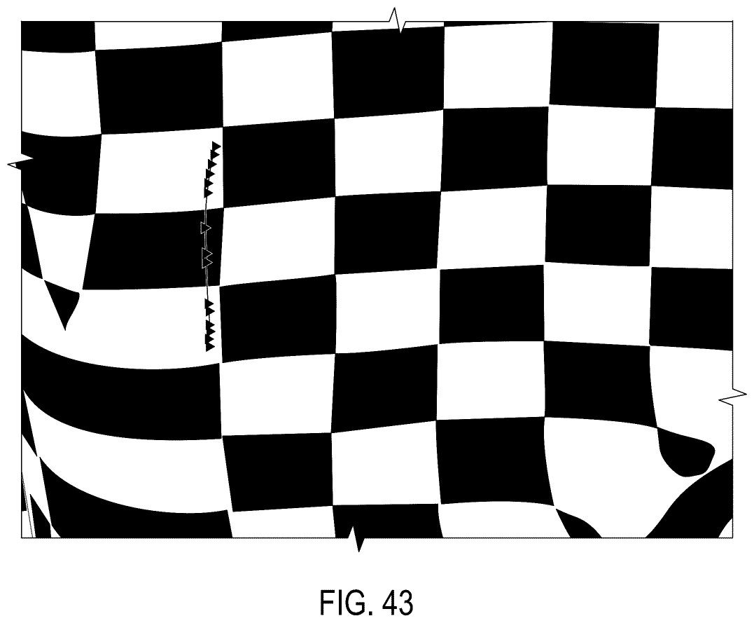

With reference now also to FIGS. 42A-43, a series of example visualizations illustrating tracking of an example region of interest are depicted. FIGS. 42A-D depict representations of various frames after detections processing (see, e.g., FIG. 30) has been completed. The detected region of interest in each frame (if present) is represented with a star. Past locations for the detected region of interest are superimposed on the frames (triangles) along with location predictions (circular dots) for the region of interest in each frame. FIG. 43 depicts the example track shown throughout FIGS. 42A-D overlaid onto a representation of a portion of an image of a bag 26.

As shown in FIG. 42A, a detected region of interest may be present in a first frame. This may result in initialization of a track (see block 3842 of FIG. 41). Track predictions may be generated and associated with detected regions of interest in subsequent frame (see blocks 3884, 3886 of FIG. 41). This may begin to create a track which documents displacement of the region of interest over the series of frames (see, e.g., FIG. 42B).

As indicated in blocks 3848, 3852 and 3856 of FIG. 41, the prediction for a region of interest may not match with a detection in a subsequent frame. In this event, predictions may continue to be generated based on the most recent known information for the region of interest associated with the track. FIG. 42B shows a prediction a number of frames after the last match for a detected region of interest. If a subsequent frame includes a detected region of interest corresponding to the prediction for that frame, the detected region of interest may be assigned to the track. In FIG. 42C, a detection is present in close proximity to the prediction for that frame and would be assigned to the example track. This may, for example, help to prevent a dark region of interest passing over a dark background section from causing a track to terminate. Additionally, it may assist in accommodating near edge filtering of a foreground segmented image. A track may, however, terminate if no detected region of interest matching a track prediction has been found for some threshold number of consecutive frames (see, e.g., block 3852 of FIG. 41). FIG. 42D depicts a track with a prediction having no matching detection the threshold number of frames since the last detected region of interest was added to the example track. As no matching detected region of interest was found in the threshold frame, the example track may be determined to be complete. This completed track is shown overlaid on an illustrative representation of an illustration of a raw image in FIG. 43. As described in FIG. 41, data corresponding to the example track would be memorialized in the tracking record dictionary, but removed from the working tracking dictionary used for generating predictions in the next frame to limit computational burden during processing.

Referring now to FIG. 44, a flowchart 3880 is depicted detailing a number of example actions which may be executed to score regions of interest associated with tracks in images of a reservoir. In the following discussion, the score is generated based off of an analysis of each frame in a given track. In other embodiments, a subset of the frames or a single frame of the track (e.g. final frame) may be utilized. In block 3882, a processor of the control system 15 may define a scoring boundary around the region of interest in each frame from a track. The scoring boundary may be a fixed and preset size. For example, a scoring boundary of not more than 20×20 pixels centered on the region of interest may be used. The size of the scoring boundary may be chosen to ensure an entire region of interest would be captured by the scoring boundary. The scoring boundary size may differ in alternative embodiments. Different size scoring boundaries may also be used depending on the size of the region interest the detection process was tuned for. For example, a larger boundary may be used if the detected region of interest was identified by a detection process tuned for extra-large regions of interest.

In block 3884, a processor of the control system 15 may analyze the pixels within the scoring boundary for each frame to determine at least one color space score. In some implementations, the processor may determine a count for the number of pixels which have color space values which fall within predefined color space value ranges (e.g. ranges of RGB values). This count may be assigned as the color space score. As mentioned in relation to FIG. 18, an external illuminator 3516 may be selected to emit light in a color (e.g. red) which is selected to differ from the color of potential sources of particulates. In example embodiments, this helps to highlight bubbles within a bag 26 or other reservoir. The color space value ranges may be specified to correspond to the light color emitted by the external illuminator 3516. Thus, scoring boundaries yielding a high color score may be suggestive that the tracked region of interest is a bubble. The predefined color space value ranges may also be selected to correspond to bright regions with a reservoir. Again, due to the optical properties of bubbles, bubbles should appear particularly bright. Regions with a high brightness score may be suggestive that the tracked region of interest is a bubble.

In block 3886, a processor of the control system 15 may analyze the pixels within the scoring boundary to determine a sharpness score. A fast Fourier transform or the variance of the Laplacian of the image within the scoring boundary could be used. Bubbles may typically be relatively sharp due to their interaction with light from the light emitter 3516. A scoring box with a high sharpness score may be indicative that the tracked region of interest is a bubble.

In block 3888, a processor of the control system 15 may determine a shape score for the region of interest over the frames of a track. The shape score may, for example, be determined based on the bounding box generated from the contours of the region of interest when the region of interest was detected (see, e.g., blocks 3680, 3682 of FIG. 31). The shape score may be determined based upon the aspect ratio of the bounding box. As bubbles are substantially spherical, they should have an aspect ratio of 1:1 (or nearly 1:1). More rectangular aspect ratios are suggestive of particulate. The aspect ratio of the shape score for a region of interest may be averaged over all frames in a given track to determine an average shape score. This in turn may be used to determine the shape score. In some examples, additional or alternative statistical processing may be performed. For example, a standard deviation of the aspect ratios for the region of interest across frames in the track may be determined. Standard deviations above a certain threshold may be indicative of rotating particulate. In certain examples, invalid shape scores may be identified and the corresponding regions of interest may be excepted from further analysis. This captures artefacts created during filtering or other image processing allowing them to be removed from the analysis.

In block 3890, scores from each analysis may be stored (e.g. written to a tracking record dictionary). A classification for the tracked region of interest may be determined based on the scores in block 3892. Certain scores may be weighted more heavily than others. For example, if a very high color space score is present, it may heavily weight the determination towards classifying the region of interest as a bubble 3550. If a shape score indicative of a highly elongate region of interest is present, it may heavily weight the determination towards classifying the region of interest as particulate.

In some embodiments, additional scores may be determined. For example, a trajectory score may be determined. The frame to frame position data for a region of interest may be analyzed to determine a direction of motion characterization for the region of interest. Velocity data may further be analyzed. The trajectory score may be an indicator of whether the region of interest appears to be sinking or rising within the bag 26. Bubbles 3550, for example, may be particularly buoyant and may tend to rise while certain types of particulate may tend to sink due to their density. Additionally, trajectory scores may be an indicator of size. For example, a larger particle of dense material would be more likely to sink (or rapidly sink).

Referring now to FIG. 45A, a log file 3590 may be generated after analysis and classification of images. An example visualization 3560 of an analysis of frame captures from an imager 3518 monitoring a bag 26 including particulate is shown in FIG. 45A. The example visualization 3560 is an annotated image. The image selected for annotation may, for example, be a final frame capture of the raw video taken by an imager 3518. In some embodiments, the images from all imagers 3518 may be stitched together (e.g. corresponding frames taken at the same time point) to from the annotated image or individual annotated images for the frame sets output by each respective imager 3518 may be generated. As described in detail above, once a region of interest in a set of frame captures has been detected, its displacement path or track within the bag 26 may be determined. The control system 15 may make a classification determination on the region of interest. As shown, each trace 3562A-C within the visualization 3560 depicts the path of a region of interest within the bag 26. The traces 3562A-C included in the visualization 3560 may be drawn from the coordinates associated with any track identities present in the tracking record dictionary (further described in relation to FIG. 41) for a set of frames. The traces 3562A-C are given different indicia to communicate their classification. Circles, squares, and triangles are used in the example, however, color, text, symbols, or other indicators may instead or additionally be used. Such visualizations 3560 may be generated for each bag 26 and placed in a log 3590 (see, e.g., FIG. 28) associated with each bag 26. A unique identifier for each trace 3562A-C (e.g. the associated track identity in the tracking record dictionary) may be superimposed over the image in the visualization 3560. The unique identifier may also be used in the log file 3590 in association with data related to the region of interest forming the respective trace 3562A-C. The log 3590 may, for instance, include the data or at least a portion of the data from the tracking record dictionary. Where a bag 26 includes no particulate, the visualization 3560 may be devoid of traces 3562A-C. The annotated image may explicitly state no tracks were identified.

Referring now also to FIG. 45B, in certain embodiments, multiple classifications may be made for the region of interest data sets collected for each bag 26. One classification may, for example, be based off classification logic which determines a classification for each region of interest based on score characteristics determined for the region of interest. Another classification may be generated by a convolutional neural network trained on images of bags 26 with known contents. The multiple classifications for each respective region of interest may be analyzed to determine a final classification. This may be a weighted determination with the output of one or more of the classifications being more controlling than others. The annotated image in FIG. 45B may be representative of the final classifications determined for a given bag 26. Thus, each individual classification could be associated with a respective annotated image and an annotated image for the final classification may be all be included in the log 3590.

In some embodiments, a second classification may only be made for regions of interest with unclear classification or low confidence classifications. For example, the traces 3562A classed with a circle in FIG. 45A may represent regions of interest with an unclear classification. The data associated with these regions of interest may be passed to secondary classification processing which may attempt to assign a classification. As shown in FIG. 45B, no traces 3562A classed with a circle are present. The traces 3562A classed with a circle have been replaced by traces 3563B-C classed with squares and triangles. Using the example of an IV bag 26, the squares may represent particulate while the triangles may represent bubbles. Thus, a second of the multiple classifications may be used to clarify any regions of interest with unclear or low confidence classification.

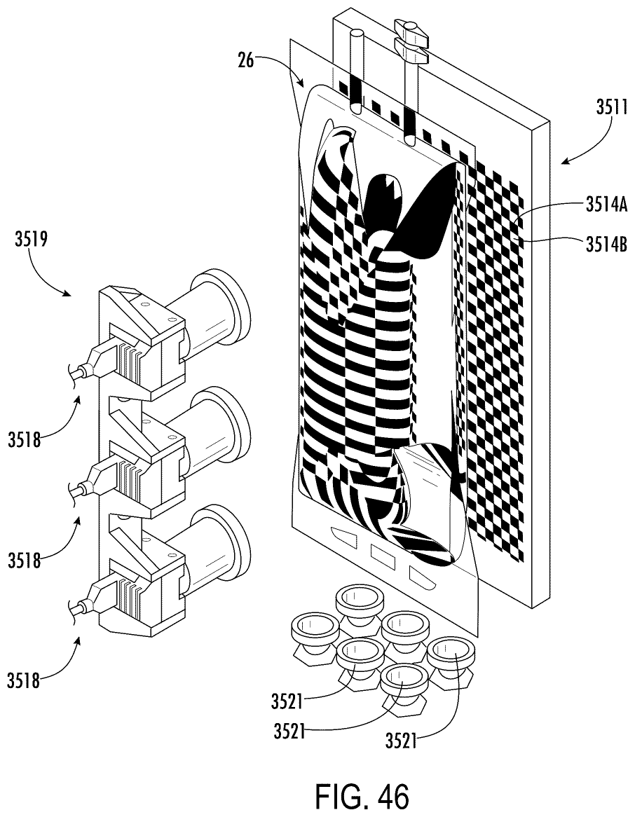

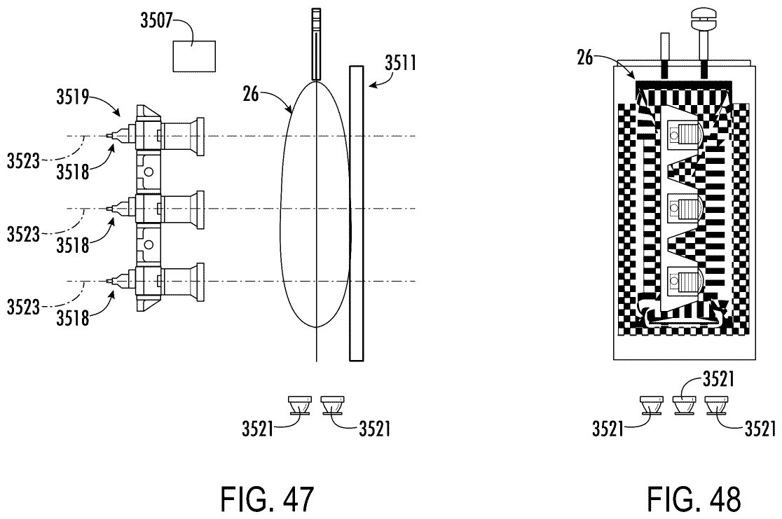

Referring to FIGS. 46-48, a portion of another example particulate inspection system 3500 is depicted. As with other particulate inspection systems 3500 described herein, the example particulate inspection system 3500 may be a fast, reliable, and inexpensive system for inspecting and distinguishing in a subject at least a first content type from a second content type. The subject may be, but is not limited to an IV bag 26. The first content type may be undesired content and the second content type may be benign content. In certain examples, the first content type may be particulate matter and the second content type may be bubbles and/or microbubbles. The content types may be in a liquid or gas contained within a subject reservoir. The example particulate inspection system 3500 includes a lighting and camera system having a plurality of imagers 3518. The example particulate inspection system 3500 may have effective detection capability over the entire subject reservoir. Example particulate inspection systems 3500 may also be robust against falsely classifying contents as an inappropriate content type.

As shown in FIGS. 46-48, an imager array 3519, at least one first light source 3521, and at least one second light source 3511 may be included. Each of the first light source 3521 may, for example, be a light emitter assembly 3522 such as those shown in relation to FIG. 19C. The second light source 3511 may, for example, be any of the illuminators 3512 shown or described in relation to FIG. 18. The second light source 3511 may include a diffuser. The second light source 3511 may back light a pattern of contrasting dark and light regions 3514A, B. This may establish contrast with respect to various contents of interest a particulate inspection system 3500 may inspect for. In an alternative embodiment, the pattern may be produced or generated with a display, such as a LED, LCD, etc. The pattern and second light source 3511 may be housed within a frame 3309. Example particulate inspection systems 3500 may also include processors (not shown in FIGS. 46-48) that control the components and analyze data therefrom.

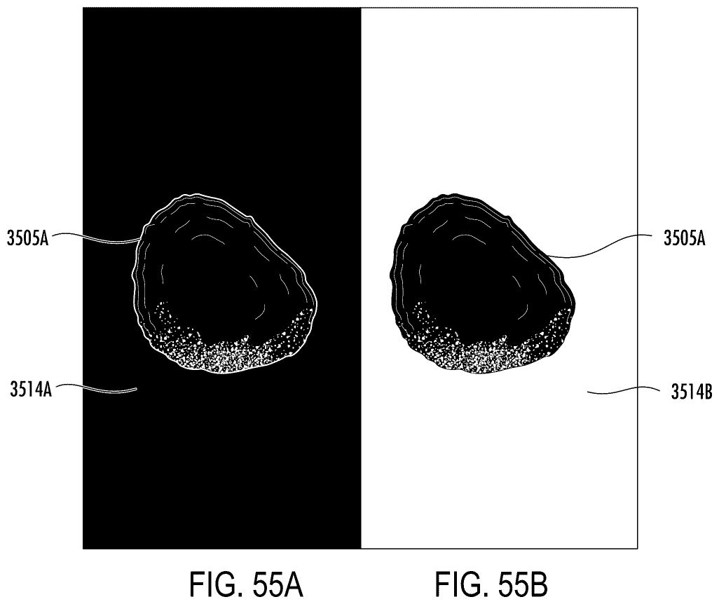

The exemplary pattern is black-and-white checkered. The exemplary pattern may permit robust detection of light, dark, reflective, transmissive, and opaque and light absorbing contents (see, e.g., FIGS. 55A-58D) within a reservoir as the contents travel across the pattern relative to view fields 3525 (see, e.g., FIG. 50) of imagers 3518. There may be an even or approximately even distribution of dark and light regions 3514A, B for observing moving objects in a subject reservoir. The anticipated velocities of contents within a reservoir and a desired frame rate for image capture may factor into the pattern and sizing of dark and light regions 3514A, B. An embodiment of the checkered pattern employs squares with sides measuring approximately 7 mm. For a given content of interest within a subject reservoir, this may provide a number of frames on each background square at anticipated average object velocities given an example frame rate of 10 frames/second. Any other contrast providing backlight or pattern described herein may be used.

In the example embodiment depicted in FIG. 46-48, a bag 26 may be interposed between imager array 3519 and the backlit pattern. The second light source 3511 may be selected and operated to optimize contrast for detecting light and dark materials in a bag 26 or other reservoir. The bag 26 may also be disposed above the at least one light source 3521. The at least one light source 3521 may project light over the entire depth and width of bag 26. Each of the at least one light source 3521 is disposed so as to project light upwardly into the bottom of bag 26. Light from each of the at least one light source 3521 may be projected substantially orthogonally to the view axis 3523 of each imager 3518. In other examples, at least one of the at least one light source 3521 may be positioned lateral to the subject reservoir and the subject reservoir may be side lit. Other positions or light sources 3521 directing light at the subject reservoir from multiple vantage points may be used in alternative examples. In certain example embodiments, an area light source 3507 may be included and may be powered to project light over the entire subject.

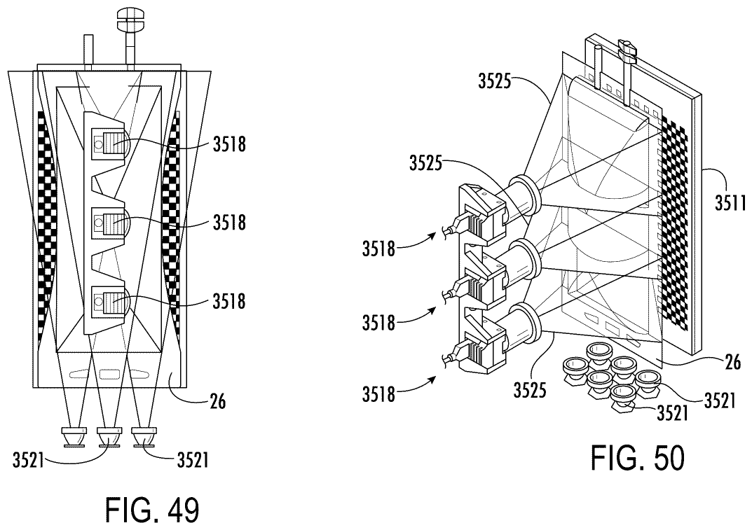

Referring now primarily to FIG. 49, in some example, the at least one light source may include multiple light emitting diodes. Each LED may be paired with a narrow beam angle lens superposed thereon. Example narrow beam angle lenses may output a 6.7° full width half max (FWHM) cone of high-intensity light though other lenses may be utilized in embodiments in which they are present. In some embodiments, each of the at least one light source 3521 may output collimated light and be paired with appropriate optics for this purpose. The color and intensity of each of the at least one light source 3521 may be selected as described elsewhere herein.

Referring now to FIG. 50, the imagers 3518 included in an imager array 3519 may be capable of capturing color image data. Each imager 3518 may have a respective view axis 3523 and a view field 3525. In certain embodiments, the imagers 3518 have a 12 MP resolution and a RGB color filter. In certain embodiments, this may be sufficient to robustly detect particles less the 200 μm across. Higher resolution imagers 3518 (e.g. 64 MP) are also possible.

In some examples, the imagers 3518 may be positioned relative to the subject such that the respective view fields 3525 are coextensive with respective areas of interest. For example, a view field 3525 of a first of the imagers 3518 may encompass at least a top third of the portion of bag 26 that contains fluid. A view field 3525 of a second of the imagers 3518 may encompass at least a middle third of the fluid-containing portion of bag 26. A view field 3525 of a third of the imagers 3518 may encompass at least a bottom third of the fluid-containing portion of bag 26. In certain examples, the imagers 3518 may be positioned relative to one another such that their respective view fields 3525 overlap. The overlap may simplify ensuring that all of the subject is captured by images collected from the imagers 3518.

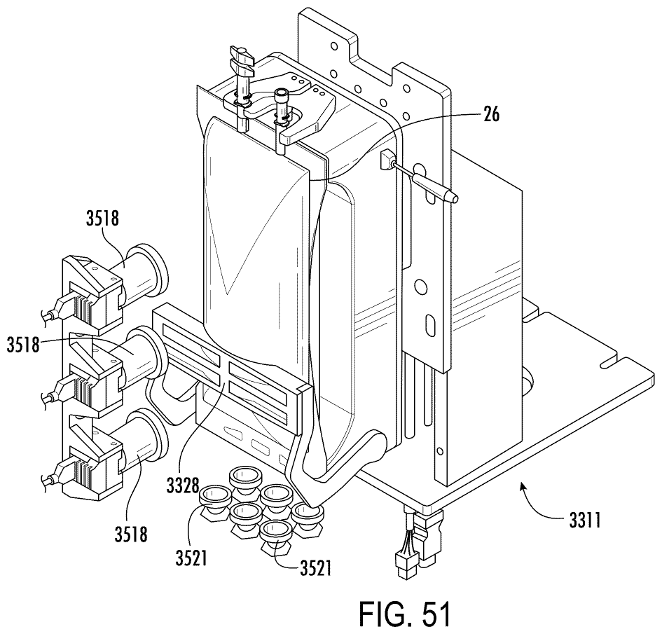

Referring to FIG. 51, shortly after a bag 26 or other reservoir is filled or agitated, fluid and any other contents therein are likely to move about within the reservoir. The bag 26 may be positioned relative to a cradle 3311 where it is maintained with a displaceable holder 3328. Any example holder described herein may be used. The bag 26 may be held in a position which retains it within the illumination field of the at least one light source 3521. Orienting the bag 26 vertically encourages air bubbles to rise perpendicularly to the view axes 3523 of the imagers 3518 against gravity. The reservoir need not be vertically oriented in all embodiments and could be horizontally oriented or at some other angle between a vertical and horizontal orientation.

In the example embodiment shown in FIG. 51, the pattern is backlit and the at least one light source 3521 may be energized to project narrow beam angle and/or collimated light up through bag 26 (or other reservoir). In some embodiments, each imager 3518 may record about ten seconds of images of the bag 26. Each imager 3518 may record image data over the same ten second period. A processor may be in data communication with the imagers 3518 and may evaluate the images. If evaluation of the images reveals no undesired contents, then the bag 26 may be labeled accordingly and distributed. If the evaluation reveals undesired contents, the bag 26 may be flagged for disposal.

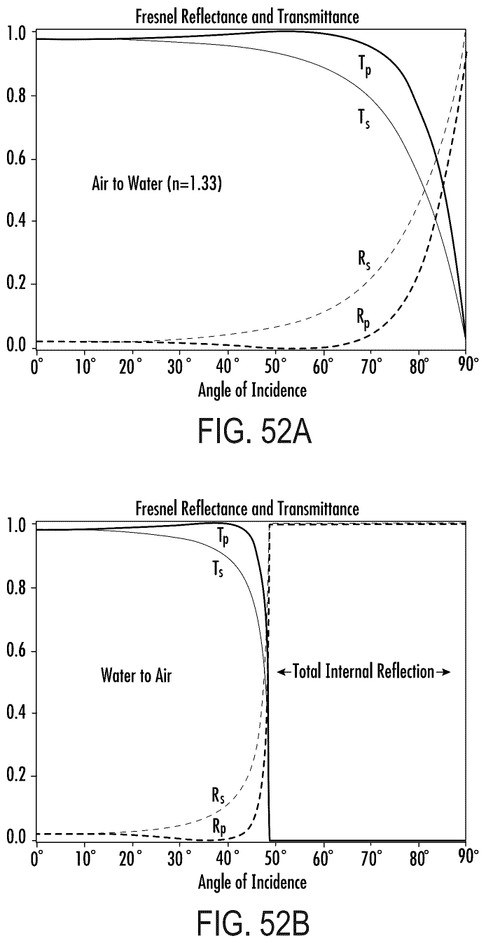

With reference now to FIG. 52A, when unpolarized light reflects off of a dielectric surface with a higher index of refraction, such as water, the reflected light may acquire or change polarization characteristics as described by Fresnel's equations. For example, sunlight reflecting off of a lake will be more polarized perpendicular to the plane of incidence (s-polarized) than polarized parallel to the plane of incidence (p-polarized) because the reflection coefficient for p-polarized light (Rp) is smaller than for s-polarized light (Rs).

With reference now to FIG. 52B, when light reflects off of the surface of a medium with a lower index of refraction than the surrounding medium, e.g. air bubbles in water, total internal reflection (T.I.R.) may be exhibited at angles of incidence greater than the critical angle. The critical angle may be computed from Snell's law and is equal to arcsin (1/1.33) or about 48.6° for air bubbles in water. When T.I.R. occurs, 100% of both s-polarized and p-polarized light are reflected back into the medium with the higher index of refraction.

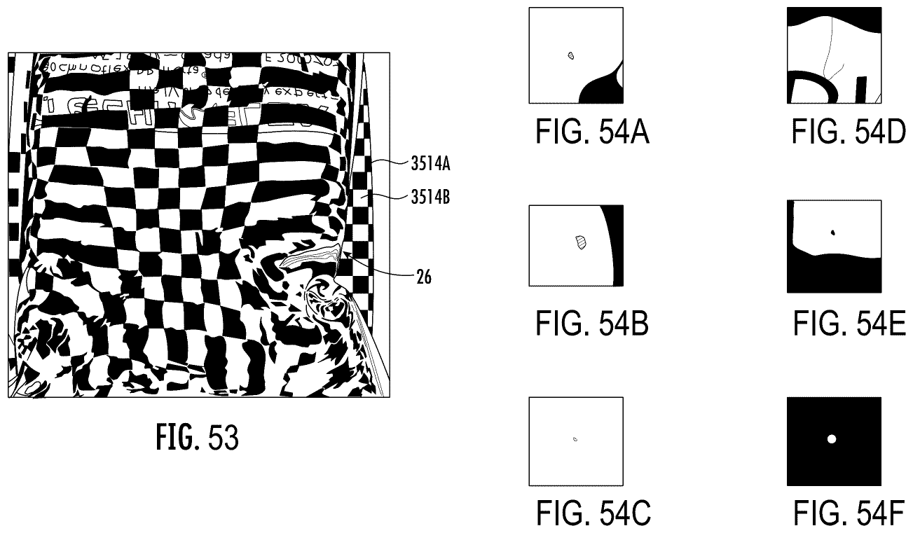

Referring now to FIG. 53, a portion of an IV bag 26 against a backlit pattern 20 that extends at least across the entire width of a bag 26 is depicted. Various content types in a bag 26 are shown in enlarged representations of images across FIGS. 54A-F. FIG. 54A depicts an enlarged portion of a representation of an image of a particle of an example septum for a bag 26 against a light region 3514B of a background pattern. FIG. 54B depicts an enlarged portion of a representation of an image of a particle of poly (methyl methacrylate) (PMMA) material (highly transparent thermoplastic) against a light region 3514B of a background pattern. FIG. 54C depicts an enlarged portion of a representation of an image of a particle of glass against a light region 3514B of a background pattern. FIG. 54D depicts an enlarged portion of a representation of an image of a fiber against light and dark regions 3415A, B of a background pattern. FIG. 54E depicts an enlarged portion of a representation of an image of a particle of black material against a light region 3514B of a background pattern. FIG. 54F depicts an enlarged portion of a representation of an image of a bubble 3550, illuminated by an example particulate inspection system 3500, against a dark region 3514A of a background pattern.

Referring to FIGS. 55A-B, a representation of a simulated image of a particle of opaque white material 3505A is shown respectively against a dark region 3514A (FIG. 55A) and a light region 3514B (FIG. 55B) of a background pattern (e.g. a chessboard pattern). As mentioned elsewhere herein, these representations, and others described herein, are not exact reproductions of actual images and are provided for illustrative purposes. The particle 3505A is simulated in a medium with an index of refraction of 1.33 (e.g. water) and itself has an index of refraction of about 1.49. The particle of opaque white material 3505A is illuminated from below by at least one light source 3521 and a second light source 3511 is illuminating the background pattern. As shown, the particle of opaque white material 3505A may be clearly visualized against both light and dark regions 3514A, B of a background pattern. The stippling generally depicts regions of the particle 3505A where light from an underlight type external illuminator 3516 is being reflected. The density of the stippling generally approximates the brightness of the reflection of light emitted by the external illuminator 3516.

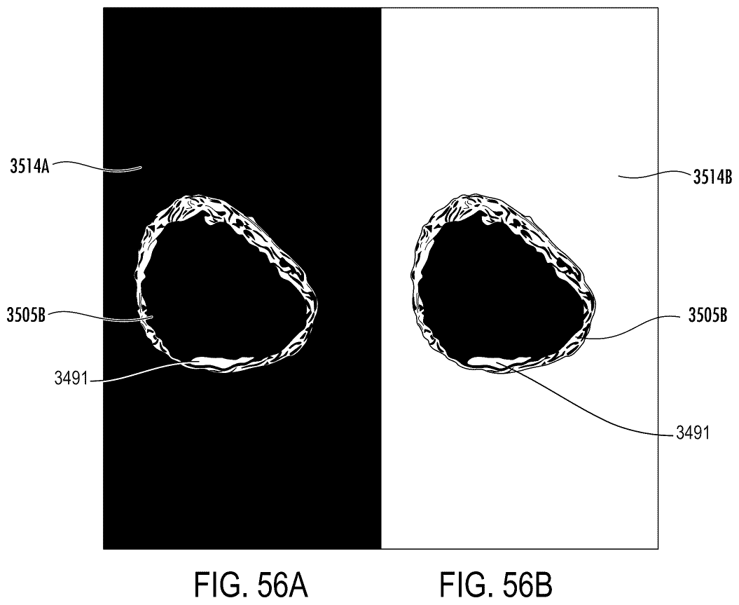

Referring now to FIGS. 56A-B, particles of reflective materials, for example, those with high albedo values (0.5-1) may also be discernable against both regions 3514A, B of a background pattern. Various metallic materials for example may be highly reflective. FIGS. 56A-B depict a simulation of an aluminum particle 3505B in a gas. The periphery of the particle 3505B displays a distortion of the background pattern with the center of the particle appearing dark. Additionally, a strong reflection 3491 of light from an underlight type external illuminator 3516 is present at the bottom of the particle.

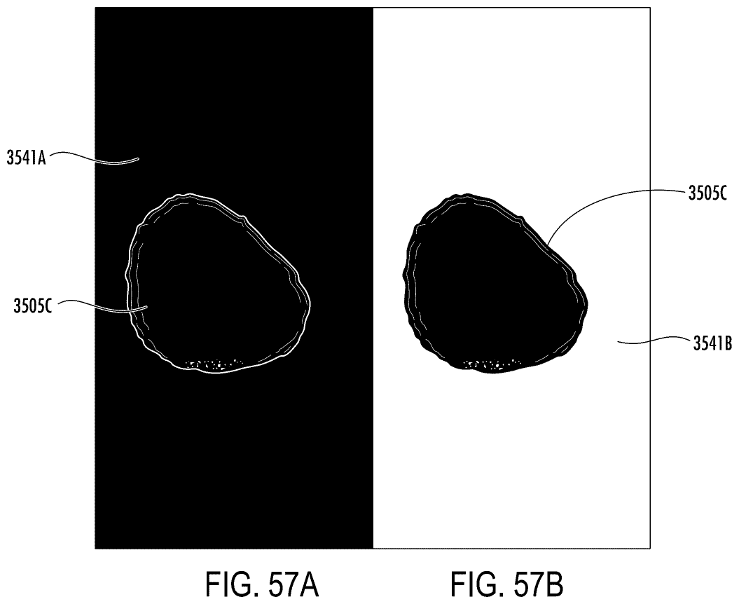

Referring now to FIGS. 57A-B, a representation of a simulated image of an illustrative particle 3505C which is fully opaque (to visible light) and which strongly absorbs visible light shining thereon is depicted.). The particle 3505A is simulated in a medium with an index of refraction of 1.33 (e.g. water) and itself has an index of refraction of about 1.49. This illustrative particle 3505C may be strongly discerned as it passes across light regions 3514B of the background pattern, but also is perceptible to a degree in front of dark regions 3514B. The stippling generally depicts regions of the particle 3505C where light from an underlight type external illuminator 3516 is being reflected. The density of the stippling generally approximates the brightness of the reflection of light emitted by the external illuminator 3516.

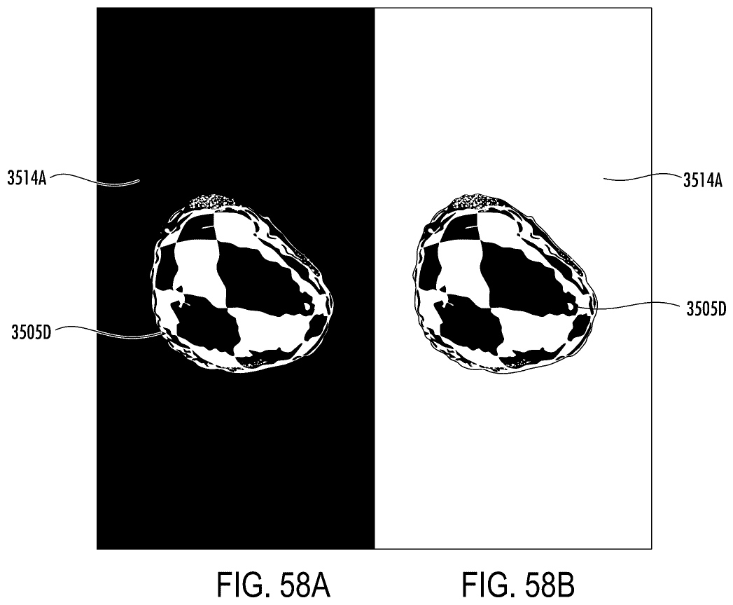

Referring now to FIG. 58A-B, a representation of a simulated image of an illustrative particle 3505D of transmissive material is depicted. Transmissive materials have less than total opacity, passing at least some light. The example shown is a clear particle 3505D of quartz in water. As shown, such particles 3505D may be clearly discerned in front of dark and light regions 3514A, B of the background pattern and generate a refracted region of distorted background pattern. The stippling generally depicts regions of the particle 3505D where light from an underlight type external illuminator 3516 is perceptible. The density of the stippling generally approximates the brightness of this light.

Referring now primarily to FIGS. 59A-60B, a bubble 3550 may be readily distinguishable from other content types by the large amount of light reflected by the bubble 3550. FIGS. 59A-B both simulate a bubble 3550 in a filled bag that reflects high-intensity light projected from the bottom of the page. FIGS. 60A-B depict illustrative representations of images of a bubble 3550 in a reservoir.

FIGS. 59A-B are from a vantage point where the line of sight extends along the medial transverse plane of the bubble 3550 and through the center of the bubble 3550. FIG. 59A is demonstrative of a bubble 3550 illuminated from below with no background pattern present. As shown in FIG. 59B and FIGS. 60A-B, reflection and refraction of a checkered pattern (e.g. backlit background pattern) is also shown. Refraction can be seen at the central region of the bubble 3550. A ring 3351A of reflection of background may surround a dark ring 3351B. The dark ring 3351 may be due to total internal reflection (T.I.R.) of light from the background pattern in this region of the bubble 3550. These rings 3351A, B may assist in distinguishing bubbles 3350 from other content types. The stippling generally depicts regions of the particle 3505D where light from an underlight type external illuminator 3516 is perceptible. The density of the stippling generally approximates the brightness of this light.

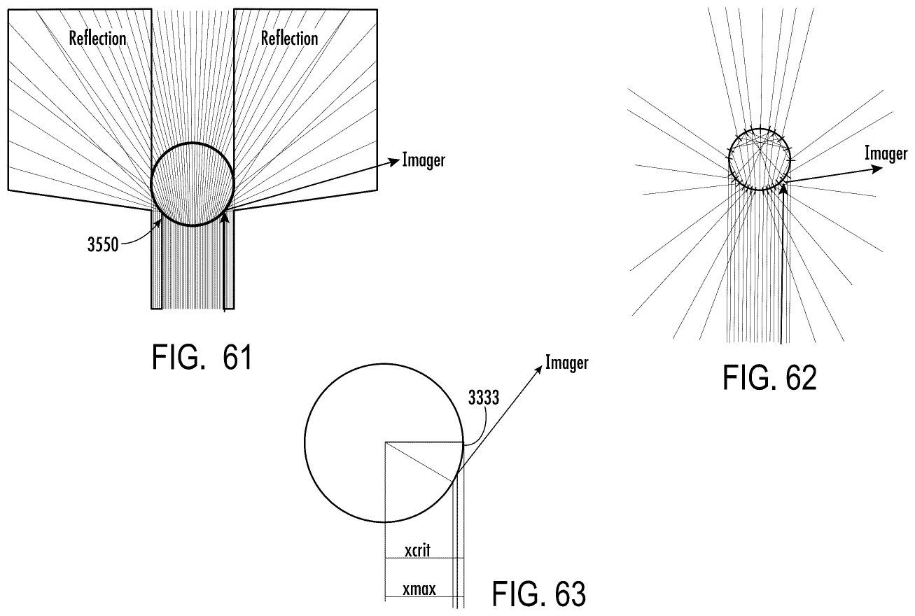

As illustrated by the diagrams in FIGS. 61-63, a bubble 3550 reflects up to 100% of incident light at angles greater than the critical angle. This is due to the index of refraction of the bubble 3550 being less than that of the surrounding medium. As shown best in FIG. 63, a ray of light reaching the bubble 3550 in a region 3333 where the angle of incidence is greater than the critical angle will reflect entirely off the surface of the bubble 3550. Consequently, reflected light from a bubble 3550 can exhibit an intensity that approaches the intensity of the light emitted by the at least one light source 3521 of FIG. 51 for instance.

In contrast, typical particulate, for example, tends to reflect only a fraction of incident light resulting in a reduced light intensity. This may be utilized to distinguish such particulate from bubbles 3350 even if the bubbles 3350 are particularly small (e.g. less than 200 μm in diameter). The maximum luminance of a particle with Lambertian surfaces may be approximated by the equation L=(ρ*E)/π. where ρ is the reflectivity of the material and E is the illuminance of light on the surface in lux.

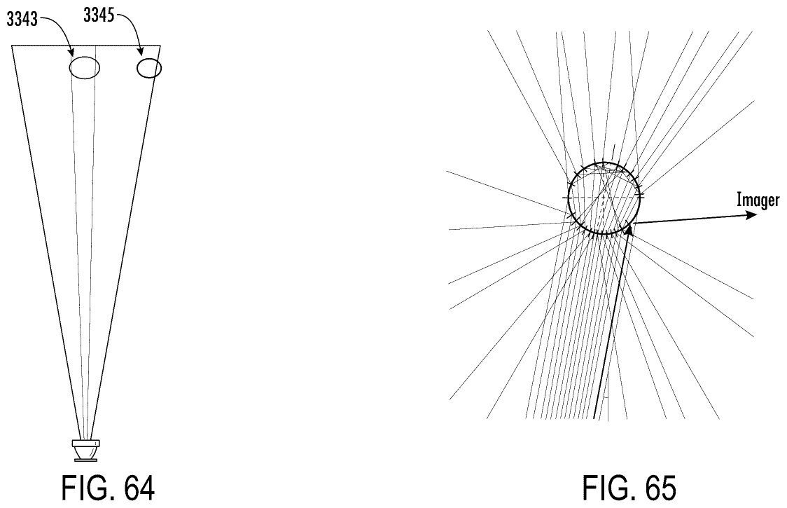

Referring now to FIGS. 64-65, a narrow beam angle and/or collimated light projecting toward a bubble 3550 generally centered in the beam (in area 3343 of FIG. 64) will behave as diagrammed in FIG. 62. At slight offset from the center of the beam (in area 3345 of FIG. 64) the narrow beam angle light projected at a bubble 3550 may have diverged to some extent and rays may not be parallel to those in FIG. 62 for example. Diverged light may behave as shown in FIG. 65.

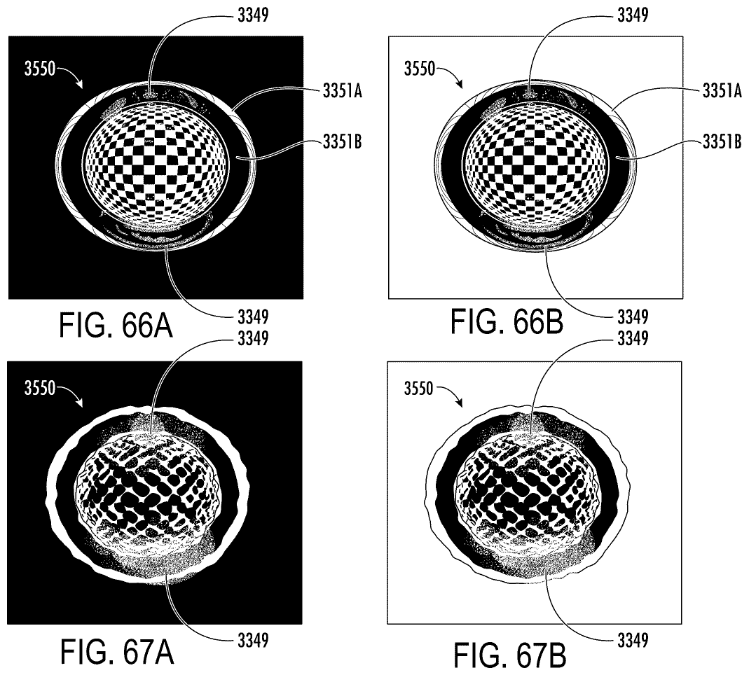

Referring to FIGS. 66A-67B, the amount of light directed to the imagers 3518 as a result of the optics of a bubble 3550 (in a medium with a higher index of refraction) allows bubbles 3350 to be robustly distinguished from other content types. It may additionally facilitate detection of bubbles 3350 in a subject reservoir which has a depth in excess of the crisp depth of field range for an imager 3518. Despite being out of focus, the bubble 3550 would remain abundantly visible. As depicted in the representations of simulated images in FIGS. 66A-B crisply in focus bubbles 3550 provide not only a high intensity bright regions 3349, but also a distorted background pattern and characteristic rings 3351A, B. If out of focus, a bubble 3550 will still be clearly observed due to the high intensity bright regions 3349 captured in images from imagers 3518. FIGS. 67A-B depict illustrative representations of simulated images of bubbles 3550 which are not in crisp focus. The high intensity bright regions 3349 and other stippling are due to interactions of the bubble 3550 with light emitted by an underlet type external illuminator 3516. The density of the stippling in FIGS. 66A-67B generally approximates the brightness of the light reaching the imager 3518 from the external illuminator.

In some embodiments of the second light source 3511 (see, e.g., FIG. 46), the second light source 3511 may emit light which is unpolarized. In other embodiments, the second light source 3511 may emit light which is polarized after passing through an associated polarizer 3503 (see, e.g., FIG. 18). At least one imager 3518 may be disposed in opposition to the second light source 3511 and each such imager 3518 may be paired with a polarizer. The polarizers associated with the second light source 3511 and the imager(s) 3518 may be oriented to provide cross-polarization. This may aid in detecting certain types of content (e.g. glass). Thus polarized back lighting with a polarizer in a cross-polarizing orientation on an imager 3518 lens may be useful for detecting quartz or other dielectric contents that change the polarization of light upon reflection or refraction.

In some example embodiments, the second light source 3511 may be paired with neutral density filter. This would decrease the intensity of projected light evenly in all wavelengths. As the light is of lower intensity, any resultant glare would also be reduced in intensity. This may afford greater control over the exposure or amount of light entering an image sensor. Exposure and/or aperture for the imagers 3518 could also be adjusted to mimic the effects of neutral density filtering.

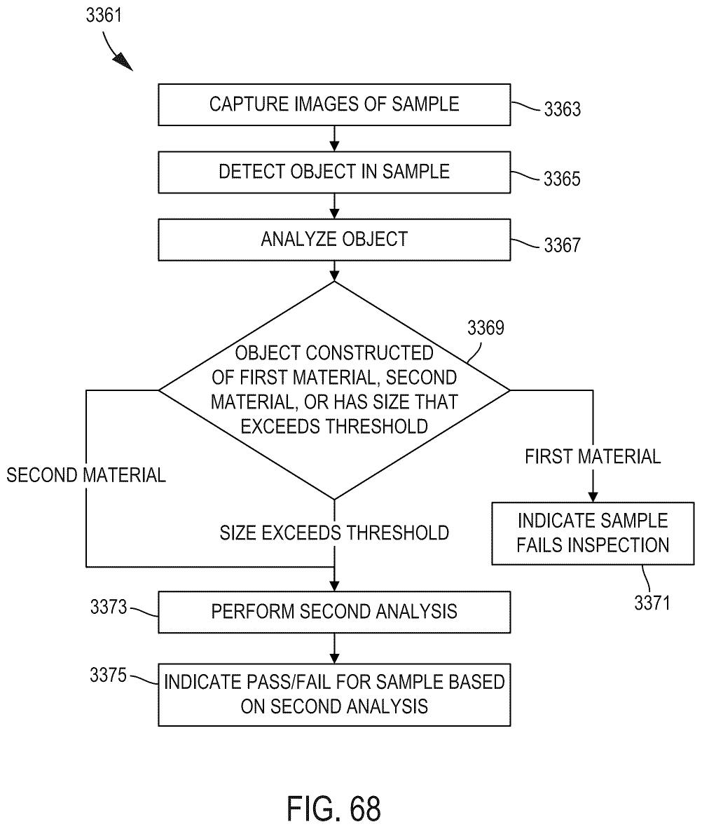

Referring now to FIG. 68, a flowchart 3361 detailing a number of example actions which may be executed to inspect a sample is depicted. Images of the sample (e.g. bag 26) may be captured in block 3363. In block 3365 objects may be detected in the image. In block 3367, detected objects may be analyzed. If, in block 3369, the analysis indicates an object is of a first material, the sample may be indicated to have failed inspection in block 3371. If, in block 3369, the analysis indicates the object is constructed of a second material, a second analysis may be performed in block 3373. Similarly, if, in block 3369, the analysis indicates that the object has a size that exceeds a threshold, a second analysis may be performed in block 3373. The sample may be assigned a pass/fail indication in block 3375 based on the second analysis.

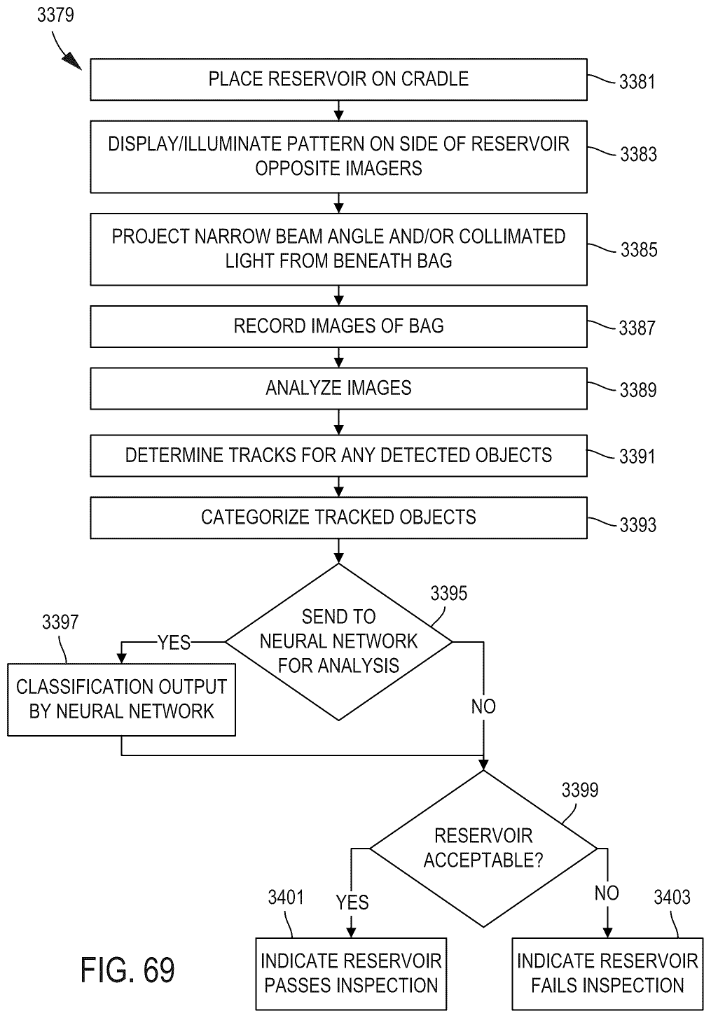

Referring now to FIG. 69, a flowchart 3379 detailing a number of example actions which may be executed to inspect a sample is depicted. In block 3381, a sample reservoir (e.g. bag 26) may be placed on a frame 3309 or cradle 3311. A pattern may be displayed or illuminated behind the sample reservoir in block 3383. In block 3385, narrow beam angle and/or collimated light may be projected from beneath the sample reservoir. Images of the sample reservoir may be recorded in block 3387. Typically the sample reservoir is agitated or mixed prior to imaging. In block 3389, the images may be analyzed. Tracks for any detected objects may be determined in block 3391. Detecting and tracking may be as described elsewhere herein. Tracked objects may be categorizing in block 3393. If, in block 3395, the object has a certain categorization, the object may be classified by a neural network in block 3397. Otherwise a determination may be made as to whether the object causes the reservoir to fail inspection without classification by the neural network. The categorization or classification from the neural network may be used to determine whether the reservoir passes or fails inspection. If, in block 3399, the reservoir is determined to be acceptable, the reservoir may be indicated as having passed in block 4301. If, in block 3399, the reservoir is determined to be unacceptable, the reservoir may be indicated as having failed inspection in block 4303.

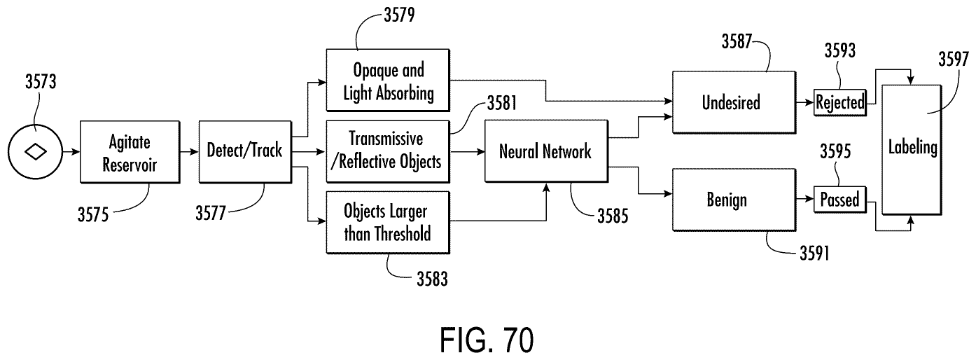

Referring now to FIG. 70, an example data flow diagram 3571 for a particulate inspection system 3500 is depicted. At block 3573, a bag 26 (or alternative reservoir type) may be placed in a particulate inspection system 3500. The bag 26 may be agitated or mixed at block 3575. The bag 26 may then illuminated by first light source(s) 3521 and a backlight for the background pattern (e.g. second light source 3511). Image data for the bag 26 may be captured by one or more imagers 3518. At block 3737, the captured images may be analyzed to detect contents of interest within the bag 26. Tracking for any detected contents of interest may also be performed. Detection and tracking of contents of interest may be as described elsewhere herein.

Detections (and potentially tracks associated with the detections) may be categorized as at least one of an opaque and light absorbing object 3579, a light transmissive or reflective object 3581, and objects having a size greater than a predefined threshold (e.g. 180 μm) 3583.

An object determined to be opaque and light absorbing with a size greater than the threshold may be classified as an undesirable content. Such objects may include free floating materials having a high opacity (e.g. at least 60% opaque to visible wavelengths) and may create moving shadows detected by background subtraction (and potentially various other processing described herein). Other objects, such as one determined to be transmissive, reflective or a sized in excess of the predefined threshold, may be classified, in some embodiments, via a neural network (block 3585). In certain embodiments, the neural network may be a convolutional neural network or CNN trained on a wide set of data pertaining to microbubble/particulate identification. The neural network may determine which detections/tracks are classified as being benign contents 3591 (e.g. bubbles 3350) and which are undesired contents 3587 (e.g. particulate). The neural net may help to limit false classification of bubbles 3550 as other, undesirable contents which would otherwise cause a bag 26 to be failed and discarded. As shown benign contents 3591 may be logged with a pass in block 3595. Reservoirs having at least one undesired content may be logged with a fail in block 3593. Bags 26 may be labeled accordingly (e.g. via a marking assembly) at block 3597.

Various alternatives and modifications can be devised by those skilled in the art without departing from the disclosure. Accordingly, the present disclosure is intended to embrace all such alternatives, modifications and variances. Additionally, while several embodiments of the present disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. And, those skilled in the art will envision other modifications within the scope and spirit of the claims appended hereto. Other elements, steps, methods and techniques that are insubstantially different from those described above and/or in the appended claims are also intended to be within the scope of the disclosure.

The embodiments shown in drawings are presented only to demonstrate certain examples of the disclosure. And, the drawings described are only illustrative and are non-limiting. In the drawings, for illustrative purposes, the size of some of the elements may be exaggerated and not drawn to a particular scale. Additionally, elements shown within the drawings that have the same numbers may be identical elements or may be similar elements, depending on the context.

Where the term “comprising” is used in the present description and claims, it does not exclude other elements or steps. Where an indefinite or definite article is used when referring to a singular noun, e.g. “a” “an” or “the”, this includes a plural of that noun unless something otherwise is specifically stated. Hence, the term “comprising” should not be interpreted as being restricted to the items listed thereafter; it does not exclude other elements or steps, and so the scope of the expression “a device comprising items A and B” should not be limited to devices consisting only of components A and B.

Furthermore, the terms “first”, “second”, “third” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

1. A particulate inspection system for detecting and classifying contents of interest within a medical reservoir comprising:
a reservoir rest body having a pattern of light and dark regions;
a rest body backlight;
an external illuminator;
a vision assembly opposite the rest body, the vision assembly including at least one imager, each imager having a field of view encompassing at least a portion of rest body; and
a controller in data communication with each of the at least one imager, the controller configured to command capture of a series of images from each of the at least one imager, receive the images, process the images into processed images, detect regions of interest within the processed images, analyze the regions of interest, and classify the regions of interest as one of a bubble and-something other than bubble.

2. The system of claim 1, wherein the system further comprises a rotary actuator coupled to the rest body.

3. The system of claim 1, wherein the rest body backlight emits white light and the pattern is a checkered pattern.

4. (canceled)

5. The system of claim 1, wherein the system further comprises a reservoir retainer container coupled to reservoir rest body, the reservoir retainer including a set of IV bag port graspers actuatable between an open state and port retaining state.

6-7. (canceled)

8. The system of claim 1, wherein the external illuminator is an underlight including a plurality of light emitters each with a narrow beam angle less than 20°, the plurality of light emitters being electrically coupled in series.

9. The system of claim 1, wherein the external illuminator includes a polarizer.

10. The system of claim 1, wherein each of the at least one imager includes a polarizing filter on a lens thereof.

11. The system of claim 1, wherein the vision assembly includes a plurality of imagers, each field of view of each imager overlapping the field of view of at least one other imager of the vision system.

12. The system of claim 1, wherein a view axis each of the at least one imager is oriented at an angle 30-35° from a plane extending perpendicular to a height axis of the rest body.

13. The system of claim 1, wherein the controller is further configured to assign each region of interest in each of the processed images to one or more track, each of the one or more track defining a displacement path of an associated region of interest throughout the processed images.

14. The system of claim 13, wherein for each track, the controller is configured to extract an extracted portion of every processed image in which the associated region of interest is detected, the extracted portion including the associated region of interest, the controller configured to analyze the regions of interest by analyzing the extracted portions.

15. The system of claim 1, wherein the controller is configured to analyze the regions of interest with a convolutional neural network.

16. The system of claim 1, wherein the controller is configured generate a reservoir acceptability determination, the acceptability determination indicated as a failure if any contents of interest are classified as something other than a bubble.

17-75. (canceled)

76. A method of capturing images of a medical reservoir for inspection comprising:
positioning a first side of the medical reservoir against a reservoir rest;
providing, with a first illuminator, a contrasting background for the medical reservoir;
illuminating the medical reservoir with a second illuminator which emits narrow beam angle, polarized light in at least one predefined wavelength range; and
capturing a plurality of images of the medical reservoir with at least one imager on a second side of the medical reservoir opposite the first side, each of the at least one imager being associated with a polarizing filter.

77. The method of claim 76, wherein positioning the first side of the medical reservoir against the reservoir rest comprises retaining the medical reservoir with a retainer assembly.

78. (canceled)

79. The method of claim 76, wherein the method further comprises agitating the medical reservoir between at least one pair of predefined rotational orientations and waiting a dwell period after agitating the medical reservoir before capturing the plurality of images.

80-81. (canceled)

82. The method of claim 76, wherein providing the contrasting background comprises backlighting the reservoir rest, the reservoir rest including a pattern of light and dark regions.

83-84. (canceled)

85. The method of claim 76, wherein illuminating the medical reservoir with a second illuminator comprises under lighting the medical reservoir with the second illuminator.

86-87. (canceled)

88. The method of claim 76, wherein the at least one imager includes a plurality of imagers, a field of view of each of the plurality of imagers overlaps the field of view at least one other imager of the plurality of imagers by at least 10%.

89. The method of claim 76, wherein the at least one imager includes a plurality of imagers and each of the plurality of imagers is oriented with a view axis at an angle 30-35° from a plane extending perpendicular to a height axis of the reservoir rest.

90. (canceled)

91. A method of verifying hardware functionality of a particulate inspection system for inspecting reservoirs comprising;
capturing a first test image of a portion of the particulate inspection system in a first condition;
performing a first comparison, with a controller, of the first test image with a first reference image and generating a first fault notification with the controller when the first comparison fails a first quality criteria,
capturing a second test image of the portion of the particulate inspection system in a second condition when the first comparison passes the first quality criteria;
performing a second comparison, with the controller, of the second test image with a second reference image and generating a second fault notification with the controller when the second comparison fails a second quality criteria;
capturing a third test image of the portion of the particulate inspection system in a third condition when the second comparison passes the second quality criteria;
performing a third comparison, with the controller, of the third test image with a third reference image and generating a third fault notification with the controller when the third comparison fails a third quality criteria; and
populating an inspection log for the reservoir with results of the first, second, and third comparison.

92. The method of claim 91, wherein the first condition is a condition in which a back light and external illuminator of the particulate inspection system are unpowered.

93. The method of claim 91, wherein the second condition is a condition in which a back light of the particulate inspection system is powered and the portion of the particulate inspection system is a patterned rest body which is illuminated by the back light.

94. The method of claim 91, wherein the third condition is a condition in which an external illuminator of the particulate inspection system is powered.

95. The method of claim 91, wherein performing the first comparison comprises aligning the first test image with the first reference image and performing a mean square error analysis with the first test image and first reference image.

96-97. (canceled)

98. The method of claim 91, wherein performing the second comparison comprises aligning the second test image with the second reference image and performing a mean square error analysis with the second test image and second reference image.

99-100. (canceled)

101. The method of claim 91, wherein performing the third comparison comprises aligning the third test image with the third reference image and performing a mean square error analysis with the third test image and third reference image.

102-103. (canceled)

104. The method of claim 91, wherein the method further comprise installing the reservoir in the particulate inspection system and capturing a fourth test image of the portion of the particulate inspection system, the method further comprising performing a fourth comparison of the fourth test image to a fourth reference image and generating a notification, with a controller, when the fourth comparison fails a fourth quality criteria.

105. The method of claim 104, wherein the fourth reference image is of the portion of the particulate inspection system with no reservoir installed in the particulate inspection system.

106. The method of claim 105, wherein performing the fourth comparison comprises aligning the fourth test image against the fourth reference image and performing a mean square error analysis, the fourth quality criteria being a mean square error threshold below which the fourth quality criteria is failed.

107-132. (canceled)
